SEQUENCE LISTING (53 sequences)

SEQ ID NO: 1 - DNA, 35 bp, Artificial Sequence
  PCR primer
cgaagaaattcgatggcttccaaaagagctctggt

SEQ ID NO: 2 - DNA, 35 bp, Artificial Sequence
  PCR Primer
cagactcgagctgcttcacatactactgctgaggt

SEQ ID NO: 3 - DNA, 20 bp, Artificial Sequence
  PCR primer
ttttggatgcaaggtcacaa

SEQ ID NO: 4 - DNA, 20 bp, Artificial Sequence
  PCR primer
ctccacaatggctagtgcaa

SEQ ID NO: 5 - DNA, 21 bp, Artificial Sequence
  Oligonucleotide for small interference RNA (siRNA)
uuccaaaccggaugaguggtt

SEQ ID NO: 6 - DNA, 21 bp, Artificial Sequence
  Oligonucleotide for siRNA
gagcgggagaccuggagcatt

SEQ ID NO: 7 - DNA, 21 bp, Artificial Sequence
  Oligonucleotide for siRNA
cuccuggaacacuggcaactt

SEQ ID NO: 8 - protein, 19 aa, Mus musculus
QNLSESPMVKEILKEQESR

SEQ ID NO: 9 - DNA, 55 bp, Artificial Sequence
  oligonucleotide
tgtcactgttgcaggcttggttcaagagaccaagcctgcaacagtgacttttttc

SEQ ID NO: 10 - DNA, 59 bp, Artificial Sequence
  Oligonucleotide
acagtgacaacgtccgaaccaagttctctggttcggacgttgtcactgaaaaaagagct

SEQ ID NO: 11 - protein, 465 aa, Homo sapiens
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETPVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDV

SEQ ID NO: 12 - DNA, 22 bp, Artificial Sequence
  PCR primer
catcagcggaaatgaccttctg

SEQ ID NO: 13 - DNA, 21 bp, Artificial Sequence
  PCR primer
cttgaagagtccacaggaacc

SEQ ID NO: 14 - DNA, 24 bp, Artificial Sequence
  PCR primer
gcaatacagcagctaccaatgctg

SEQ ID NO: 15 - DNA, 24 bp, Artificial Sequence
  PCR primer
ggctaagatctacagctgccttgt

SEQ ID NO: 16 - DNA, 25 bp, Artificial Sequence
  PCR primer
catggactacaaggacgacgatgac

SEQ ID NO: 17 - DNA, 24 bp, Artificial Sequence
  PCR primer
aatcggagctgtattctcccgaag

SEQ ID NO: 18 - DNA, 24 bp, Artificial Sequence
  PCR primer
ttgaaaaactctccacggtgtact

SEQ ID NO: 19 - DNA, 24 bp, Artificial Sequence
  PCR primer
tacgggtcaaacttcacagagaat

SEQ ID NO: 20 - DNA, 21 bp, Artificial Sequence
  PCR primer
gtatgctctgatgccatccat

SEQ ID NO: 21 - DNA, 21 bp, Artificial Sequence
  PCR primer
tgatgcacatagcagcaactc

SEQ ID NO: 22 - DNA, 19 bp, Artificial Sequence
  PCR primer
ctccctccatgagggtctg

SEQ ID NO: 23 - DNA, 20 bp, Artificial Sequence
  PCR primer
tcttcggcttcgagggtaaa

SEQ ID NO: 24 - DNA, 20 bp, Artificial Sequence
  PCR primer
tttcgcaacgggtttgccgc

SEQ ID NO: 25 - DNA, 24 bp, Artificial Sequence
  PCR primer
aaggtcgcctctagcttcctgtag

SEQ ID NO: 26 - DNA, 24 bp, Artificial Sequence
  PCR primer
aatatctacggactcatcgctccc

SEQ ID NO: 27 - DNA, 24 bp, Artificial Sequence
  PCR primer
gcatacacactcccatagacagac

SEQ ID NO: 28 - DNA, 22 bp, Artificial Sequence
  PCR primer
gccggggctgcgcttggctttg

SEQ ID NO: 29 - DNA, 24 bp, Artificial Sequence
  PCR primer
tggcggtgcttccgggacatgagc

SEQ ID NO: 30 - DNA, 24 bp, Artificial Sequence
  PCR primer
gaaactctccctgactctactccc

SEQ ID NO: 31 - DNA, 20 bp, Artificial Sequence
  PCR primer
cttgctgctcttcctctggt

SEQ ID NO: 32 - DNA, 21 bp, Artificial Sequence
  PCR primer
aactccaccctcatcaaccca

SEQ ID NO: 33 - DNA, 21 bp, Artificial Sequence
  PCR primer
aagcccagacctttcaggaca

SEQ ID NO: 34 - DNA, 24 bp, Artificial Sequence
  PCR primer
tccaaagggaacaactgcctcgat

SEQ ID NO: 35 - DNA, 22 bp, Artificial Sequence
  PCR primer
agctgtgcttggctggaacttt

SEQ ID NO: 36 - DNA, 22 bp, Artificial Sequence
  PCR primer
acgcttggtggttcattctgga

SEQ ID NO: 37 - DNA, 24 bp, Artificial Sequence
  PCR primer
aacttgcttcctgtttctgagggc

SEQ ID NO: 38 - DNA, 22 bp, Artificial Sequence
  PCR primer
acgacatcactggctgacactt

SEQ ID NO: 39 - DNA, 22 bp, Artificial Sequence
  PCR primer
tccaaaccgacttgactgctga

SEQ ID NO: 40 - DNA, 24 bp, Artificial Sequence
  PCR primer
tgagagggaaatcgtgcgtgacat

SEQ ID NO: 41 - DNA, 24 bp, Artificial Sequence
  PCR primer
accgctcgttgccaatagtgatga

SEQ ID NO: 42 - DNA, 24 bp, Artificial Sequence
  PCR primer
aagctgaccctgaagttcatctgc

SEQ ID NO: 43 - DNA, 24 bp, Artificial Sequence
  PCR primer
caccttgatgccgttcttctgctt

SEQ ID NO: 44 - DNA, 19 bp, Artificial Sequence
  PCR primer
ttcgcaacgggtttgccgc

SEQ ID NO: 45 - DNA, 23 bp, Artificial Sequence
  PCR primer
aaggtcgcctctagctcctgtag

SEQ ID NO: 46 - DNA, 24 bp, Artificial Sequence
  PCR primer
tggcacagtccctatttcttcgct

SEQ ID NO: 47 - DNA, 24 bp, Artificial Sequence
  PCR primer
aatgcagccaatagcaccacacag

SEQ ID NO: 48 - DNA, 24 bp, Artificial Sequence
  PCR primer
ggcgtatggagcagggttactttc

SEQ ID NO: 49 - DNA, 22 bp, Artificial Sequence
  PCR primer
aatgctctgcgtgtagcggttg

SEQ ID NO: 50 - DNA, 24 bp, Artificial Sequence
  PCR primer
agcgagccttgttgacatgagaga

SEQ ID NO: 51 - DNA, 24 bp, Artificial Sequence
  PCR primer
tcaagattgcttggcttggttggg

SEQ ID NO: 52 - DNA, 24 bp, Artificial Sequence
  PCR primer
tcaacgttaagctagccctggaca

SEQ ID NO: 53 - DNA, 24 bp, Artificial Sequence
  PCR primer
tctgtacaggaatggtgatgcggt

(19) United States
(12) Patent Application Publication
(10) Pub. No.: US 2006/0171935 A1
(43) Pub. Date: Aug. 3, 2006

(54) Protecting cell therapy for neurological disorders including Parkinson's disease

(76) Inventors: Asa Abeliovich, New York, NY (US); Cecile Martinat, New York, NY (US)

Correspondence Address: WILMER CUTLER PICKERING HALE AND DORR LLP;COLUMBIA UNIVERSITY, 399 PARK AVENUE, NEW YORK, NY, 10020, US

(21) Appl. No.: 11/048,391

(22) Filed: Jan. 31, 2005

(51) Int. Cl. A61K 48/00; C12N 5/08

(52) U.S. Cl. 424/93.21; 435/368

(57) ABSTRACT

The present invention provides compositions and methods for enhancing and improving cell therapies for neurological disorders including Parkinson's disease. Specifically, the present invention provides a cell or cells modified by viral vector compositions that allow for the over-expression and RNAi mediated knockdown of genes in vitro and in vivo. The present invention further provides cell therapy methods for treating or preventing neurodegeneration in a subject, and for protecting neurons from damage in the context of neurodegenerative disorders using the modified cells. Additionally, the present invention provides methods for purification and identification of mature dopamine neurons for cell therapy using fluorescent compounds including JHC1-64.

### BACKGROUND OF THE INVENTION

Parkinson's disease (PD) is a progressive, neurodegenerative disease, the symptoms of which include tremors, speech impediments, movement difficulties, and dementia. The pathological hallmark of PD is the relatively selective loss of dopamine neurons (DNs) in the substantia nigra pars compacta in the ventral midbrain. As a consequence, dopamine is deficient in Parkinson's patients. Although the cause of neurodegeneration in PD is unknown, a Mendelian inheritance pattern is observed in approximately 5% of patients, suggesting a genetic factor. Extremely rare cases of PD have been associated with the toxin 1-methyl-4-phenyl-1,2,3,6-tetrahydropyridine, which is taken up specifically by DNs through the dopamine transporter and is thought to induce cellular oxidative stress. Population-based epidemiological studies have further supported roles for genetic and environmental mechanisms in the etiology of PD (Dauer and Przedborski 2003; Jenner 2003).

Recent studies indicate that two separate mutations in the gene coding for alpha-synuclein are responsible for certain rare familial forms of PD. More recent epidemiological studies indicate that parkin is also defective in a significant percentage of all familial PD. Deprenyl (selegiline) may slow progression of PD, if it is begun early in the disorder. There is also evidence that antioxidants, such as selenium and vitamin E, may be of some benefit. Nevertheless, there is still no known cure for PD, and a need exists for compositions and methods of treatment for PD and related neurological disorders.

The identification of several genes that underlie familial forms of PD has allowed for the molecular dissection of mechanisms of DN survival. Autosomal dominant mutations in α-synuclein lead to a rare familial form of PD (Polymeropoulos et al. 1997), and there is evidence that these mutations generate toxic, abnormal protein aggregates (Goldberg and Lansbury 2000) and cause proteasomal dysfunction (Rideout et al. 2001). A majority of patients with sporadic PD harbor prominent intracytoplasmic inclusions, termed Lewy bodies, enriched for α-synuclein (Spillantini et al. 1998), as well as neurofilament protein (Trojanowski and Lee 1998). Mutations in a second gene, Parkin, lead to autosomal recessive PD (Hattori et al. 2000). Parkin is an ubiquitin ligase that appears to participate in the proteasome-mediated degradation of several substrates (Staropoli et al. 2003).

Homozygous mutations in a third gene, DJ-1, were recently associated with autosomal recessive primary parkinsonism (Bonifati et al. 2003). DJ-1 encodes a ThiJ domain protein of 189 amino acids that is broadly expressed in mammalian tissues (Nagakubo et al. 1997). Interestingly, DJ-1 was independently identified in a screen for human endothelial cell proteins that are modified with respect to isoelectric point in response to sublethal doses of paraquat (Mitsumoto and Nakagawa 2001; Mitsumoto et al. 2001), a toxin that generates reactive oxygen species (ROS) within cells and has been associated with DN toxicity (McCormack et al. 2002). Gene expression of a yeast homolog of DJ-1, YDR533C, is upregulated in response to sorbic acid (de Nobel et al. 2001), an inducer of cellular oxidative stress. These results suggest a causal role for DJ-1 in the cellular oxidative stress response.

Surprisingly, animal models that harbor genetic lesions that mimic inherited forms of human PD, such as homozygous deletions in parkin (Goldberg et al. 2003; Itier et al. 2003) or overexpression of α-synuclein (Masliah et al. 2000; Giasson et al. 2002; Lee et al. 2002), have failed to recapitulate the loss of dopamine cells. An alternative approach, the genetic modification of midbrain DNs in vitro (Staropoli et al. 2003), is potentially useful but limited by the difficulty and variability in culturing primary postmitotic midbrain neurons. Other studies have focused on immortalized tumor cell lines, such as neuroblastoma cells, but these may not accurately model the survival of postmitotic midbrain neurons. Thus, a major limitation in the prevention and treatment of PD is the lack of reliable animal and cellular models for the disease. Accordingly, there exists a need for improved cellular and animal models of PD and other neurodegenerative disorders.

Although symptomatic therapies exist for Parkinson's disease (PD) that improve the motor function of patients, no treatments are available that slow the relentless course of the disease. Given the relatively specific loss of dopamine neurons (DNs) in PD, cell replacement therapies offer a promising treatment strategy (Dauer and Przedborski, 2003). However, major hurdles remain: the current state-of-the-art in dopamine cell therapy is of limited efficacy. In two placebo-controlled, prospective trials with fetal-derived midbrain cells transplanted into the striatum, patients experienced no subjective benefit, although some younger patients did appear to improve by certain objective measures (Freed et al., 2000; Olanow et al., 2003). A significant percentage of treated patients suffered from dyskinesias in both studies. These results suggest that transplanted cells require further cues to function in the context of an intact CNS, and emphasize the importance of identifying critical developmental signals for dopamine neurons. Accordingly, a need exists for improved gene therapy and cell therapy based treatments for PD and other neurodegenerative disorders.

A critical issue with regard to cell replacement therapy is the availability of appropriate donor cells. Fetal-derived dopamine neurons have been used in most of the previously attempted cell replacement clinical studies, but such cells are of limited availability and are subject to ethical debate. In contrast, stem cell-derived dopamine neurons, either from embryonic stem (ES) cells or from neuronal stem cells (NSC) offer the potential for a limitless supply, as stem cells by definition are self-renewing (Freed, 2002).

Embryonic stem cells (ES cells), derived from early embryos, are “immature” cells that have the potential to develop into different cell types including DNs (Bjorklund et al., 2002; Kim et al., 2002). The in vitro differentiation of ES cells provides new perspectives for studying the cellular and the molecular mechanisms of neuronal development. Murine ES-derived dopamine neurons have been shown to follow much the same early differentiation pattern as endogenous dopamine neurons with respect to a number of early markers. Furthermore, transplantation of murine ES-derived dopamine neurons appear to function in an animal model of PD, 6-hydroxydopamine treated rats.

Several studies have investigated the role of ‘early’ extrinsic factors, including Sonic Hedgehog and FGF8, in ES differentiation protocols, and these suggest that these factors do potentiate the generation of DNs (Kim et al., 2002; Lee et al., 2000). Furthermore, overexpression of Nurr1, an ‘early’ intrinsic factor, appears to potentiate the generation of early markers of DNs (Chung et al., 2002; Kim et al., 2002), particularly TH. One caveat to the interpretation of the study from Kim et al., however, is that they do not compare the Nurr1-transfected ES clone to control vector-transfected cells, limiting the interpretation of these data. One study (Rolletschek et al., 2001) did investigate the efficacy of a cocktail of growth factors (including BDNF and GDNF) on the maturation of ES-derived dopamine neurons, but this study failed to observe an effect on dopamine levels of this cocktail, and did not include a kinetic analysis of the roles of these factors.

It remains of great interest to define the subsequent regulatory events that specify the late differentiation of ES-derived dopamine neurons, and to relate these later events to the earlier factors. A detailed analysis of late events in the development of functional ES-derived dopamine neurons is likely lead to improvements in cell therapies. Cells transplanted into adult CNS are clearly influenced by the local environment, and this will differ from the normal developing midbrain DN environment. It is possible that such improvements will impact both the survival and function of the transplanted cells. Also, a potential consideration in the differentiation of ES cells is that poorly differentiated ES-derived transplants can lead to tumor formation (Chung et al., 2002).

### SUMMARY OF THE INVENTION

Cell therapies for PD have been available over the past two decades, but in clinical placebo controlled trials, these therapies have not performed any better than the available drugs, which fail to slow the progression of the disease. Available cell therapies are likely to be insufficiently mature and survive poorly in vivo. The present invention discloses the development of viral vectors that can be used to modify cell-based therapies in order to improve their efficacy.

Current knowledge regarding the mechanism of action of Parkinson's disease and Alzheimer's disease, along with novel technical advances, allow for new approaches to these disorders. The inventors have previously described that transduction of either of two genes, Parkin and DJ-1, into midbrain dopamine neurons, leads to significant protection in vitro in primary neuronal culture systems. The inventors disclose the development of viral vectors that allow for the over-expression and RNAi mediated knockdown of particular genes both in vitro and in vivo in a subject. The present invention discloses use of these viral vectors to genetically enhance cell based therapies. In an embodiment of the present invention, ES-derived dopamine neurons are modified by the viral vectors.

The inventors disclose herein that these viral vectors, as well as cells and cell therapies modified by the vectors, can be utilized as therapeutic agents in the context of treating or preventing neurodegeneration, including Parkinson's disease, in a subject. In an embodiment of the invention, ES cells transduced by the viral vectors are administered to a subject by stereotaxic injection into the ventral midbrain of the subject. Additionally, the viral vectors of the invention can also be used for modifying existing cellular and animal models of neurodegeneration to overcome limitations in these model systems. Parkin and DJ-1 have previously been identified as genes that, when lost or defective, lead to Parkinson's disease. The inventors' findings indicate that overexpression of these genes leads to the opposite effect—i.e., protection from toxins. The inventors describe the overexpression of Nurr1 and PitX3 using this system. The inventors further describe the overexpression of a new PD gene using this system—Pink1—as well as the knockdown of toxic genes for Parkinson's disease (alpha Synuclein) and for Alzheimer's disease (amyloid precursor protein (APP)).

Accordingly, in one aspect, the present invention provides a cultured cell or cells transduced by a composition comprising a viral vector that allows for the overexpression of specific genes and homologs thereof in vivo that protect neurons from toxins. In a specific embodiment, the invention provides a cultured cell transduced or modified by a composition comprising a nucleic acid encoding a Nurr-1 associated agent and a PitX3-associated agent; a vector; and optionally, a pharmaceutically-acceptable carrier; wherein the Nurr-1-associated agent is selected from the group consisting of a Nurr-1 protein, a Nurr-1 mimetic, a modulator of Nurr-1 expression and a modulator of Nurr-1 activity; and the PitX3-associated agent is selected from the group consisting of a PitX3 protein, a PitX3 mimetic, a modulator of PitX3 expression and a modulator of PitX3 activity. In other specific embodiments of the present invention, the nucleic acid of the composition encodes a parkin-associated agent, a pink-1 associated agent, a DJ-1-associated agent.

The present invention further provides cells transduced or modified by compositions comprising viral vectors that allow for RNAi mediated knockdown of specific genes toxic to neurons. In one embodiment, the invention provides a cell or cells transduced by a composition comprising a nucleic acid having a sequence sufficiently complementary to a portion of an alpha synuclein gene to reduce expression of the gene a vector; and optionally, a pharmaceutically-acceptable carrier wherein the nucleic acid is selected from the group consisting interfering RNA, and shRNA. In another embodiment of the invention, the cell or cell is transduced by a composition comprising a nucleic acid having a sequence sufficiently complementary to a portion of an amyloid precursor protein gene to reduce expression of the gene.

In another embodiment of the invention, the vector of the therapeutic composition also expresses a fluorescent protein, such as green fluorescent protein (GFP). In a specific embodiment, the vector of the therapeutic composition expresses eGFP.

The cells transduced by the vectors of the present invention include but are not necessarily limited to ES cells, neuronal precursor cells, ES-derived dopamine neurons, dopamine neuron precursors and midbrain dopamine neurons.

The present invention additionally provides methods for treating or preventing neurodegeneration in a subject in need of such treatment by administering to the subject the transduced cells of the present invention in an amount effective to treat or prevent the neurodegeneration. In one embodiment of the invention, the cells are administered with a pharmaceutically acceptable carrier. The neurodegeneration or neurodegenerative disorders treated or prevented by the method of the present invention include, but are not necessarily limited to Parkinson's disease (including sporadic Parkinson's disease and autosomal recessive early-onset Parkinson's disease), Alzheimer's disease, stroke, amyotrophic lateral scelerosis, Binswanger's disease, Huntington's chorea, multiple sclerosis, myasthenia gravis, and Pick's disease. In a preferred embodiment of the invention, the neurodegeneration treated or prevented by the compositions and methods of the present invention is Parkinson's disease. In one embodiment of the invention, the cell therapy composition of the invention is used in combination with one or more different cell therapy compositions of the present invention.

In a specific embodiment of the invention, the cell therapy composition is administered directly into the brain of a subject. The cell compositions of the present invention can be directly administered to any structure in the brain. In one embodiment, the compositions are administered to brain structures selected from the group consisting of ventral midbrain, substantia nigra, hippocampus, striatum, and cortex. In a preferred embodiment of the invention, the composition is administered using a stereotactic device.

Also provided are methods for use of the cell compositions of the invention to improve an animal model of Parkinson's disease or other neurodegenerative disorder. In one embodiment, a cell composition is provided, wherein the cell have been modified by composition comprising a nucleic acid comprising a sequence sufficiently complementary to a portion of a gene selected from the group consisting of PAD1, Psmc4, Apg7L and NPC, to reduce expression of the gene a vector; and optionally, a pharmaceutically-acceptable carrier wherein the nucleic acid is selected from the group consisting interfering RNA, and shRNA. In another embodiment, the vector expresses a fluorescent protein, including but not limited to green fluorescent protein, and the vector is selected from the group consisting of an adeno-associated viral vector or a lentiviral vector. In another embodiment, the animal model of neurodegeneration or neurodegenerative disorder includes but is not necessarily limited to Parkinson's disease, autosomal recessive early-onset Parkinson's disease, Alzheimer's disease, stroke, amyotrophic lateral scelerosis, Binswanger's disease, Huntington's chorea, multiple sclerosis, myasthenia gravis, and Pick's disease.

The inventors further disclose herein that fluorescent dye JHC 1-64 (C₅₀H₆₁Cl₂N₅O₉S₂) (FIG. 48), when used in conjunction with Fluorescent Activated Cell Sorting (FACS), is valuable for identifying mature dopamine neurons and purifying mature dopamine neurons from immature ES cells or other populations in cell therapies. This discovery is critical for successful cell therapies because immature or otherwise contaminating cells can lead to tumors. Purification of mature cells leads to more efficacious treatment. Accordingly, the present invention also provides methods for preparing a preparation of mature dopamine neurons comprising contacting a cell sample with at least one fluorescent compound that targets the dopamine transporter protein; identifying cells containing the dopamine transporter protein, and removing cell and cellular aggregates that do not contain the dopamine transporter protein by FACS analysis; wherein the resulting preparation consists essentially of cells containing the dopamine transporter protein. A combination of more than one fluorescent compound can be used in the method of the invention. In a preferred embodiment, at least one of the fluorescent compounds is JHC 1-64. By way of non-limiting example, the cell sample may contain embryonic stem (ES) cells, neuronal precursor cells, ES derived dopamine neurons, dopamine neuron precursor cells, and dopamine neurons.

Additional aspects of the present invention will be apparent in view of the description, which follows.

### BRIEF DESCRIPTION OF THE FIGURES

FIG. 1 illustrates that parkin interacts specifically with the F-box/WD-repeat protein, hSel-10. (A) Flag-Parkin (52 kDa) was co-expressed with Myc-hSel-10 (69 kDa), Myc-UbcH7 (18 kDa), or PP2A/Bα (55 kDa), in HeLa cells. Anti-Flag immunoprecipitates and lysates were probed, as indicated, by Western blotting. The asterisk indicates the position of an immunoglobulin light chain. (B) Insect cells were co-infected with baculovirus expressing GST-parkin (75 kDa), Flag-hSel-10 (110-kDa form), or Flag-β-TrCP (65 kDa). GST pull-downs, or anti-Flag immunoprecipitations, were performed as described, followed by Western blotting with monoclonal antibodies to either the Flag tag or the parkin ubiquitin homology domain (FIGS. 9-11). (C) The primary structures of parkin and hSel-10, showing their major domains, are presented. (D) Either wild-type parkin, ARPD mutant (T240R) parkin, a deletion mutant form of parkin lacking the ubiquitin homology domain (ΔUHD parkin), or a truncated form of parkin corresponding to its UHD alone (parkin^{UHD}), was co-expressed in HeLa cells with Myc-tagged hSel-10 (wild-type, mutant WD-repeat alone (hSel-10^{WD}, 49 kDa) or mutant F-box alone (hSel-10^{F-box}, 35 kDa)). Anti-Myc immunoprecipitates and crude lysates were analyzed by Western blotting with polyclonal antibodies to Myc or to the carboxyl terminus of parkin. The parkin polyclonal antibody recognizes both full-length parkin (52 kDa) and a truncated form that is deleted in the UHD (ΔUHD; 42 kDa), and appears to be generated by post-translational processing (Schlossmacher et al., Parkin localizes to the Lewy bodies of Parkinson disease and dementia with Lewy bodies. Am. J. Pathol., 160:1655-67, 2002) (data not shown). (E) Homogenates of 1-g frozen frontal cortex from an ARPD case (see Examples) or an age-matched control were immunoprecipitated with a monoclonal antibody specific for the amino-terminus of human parkin (FIGS. 9-11), and probed for parkin (using this parkin monoclonal antibody), hSel-10, or α-synuclein. (F) Fresh mouse brain (2 g total) homogenates were incubated with Flag-β-TrCP produced in insect cells or immobilized recombinant Flag-hSel-10 (110 kDa). The 69-kDa and 110-kDa forms of hSel-10 both contain the F-box and WD-repeat domains (Koepp et al., Phosphorylation-dependent ubiquitination of cyclin E by the SCFFbw7 ubiquitin ligase. Science, 294:173-77, 2001). Complexes were Flag-immunoprecipitated and probed by Western blotting for cyclin E (51 kDa) or parkin (using the parkin monoclonal antibody). The asterisk indicates the position of an immunoglobulin heavy chain.

FIG. 2 shows that hSel-10 and UbcH7 function co-operatively to potentiate parkin ubiquitin ligase activity. (A) Plasmids encoding Flag-WT or T240R ARPD parkin were co-transfected, with or without hSel-10 (69-kDa), into HeLa cells. The cells were subsequently treated with lactacystin, to inhibit proteasome function. Cell lysates were immunoprecipitated with an anti-Flag antibody, and probed by Western blotting. (B) Flag-parkin and HA-ubiquitin were co-transfected in HeLa cells, along with full-length hSel-10 (69 kDa), hSel-10^{WD}, hSel-10^{F-box}, β-TrCP, or vector. Lysates were immunoprecipitated with anti-Flag antibody, and ubiquitinated species were detected by Western blotting with an anti-HA antibody. Autoradiography exposure time was extended in (B), relative to the other panels (cf. (B), lane 1; (C), lane 1), to allow for detection of the lower levels of auto-ubiquitination observed in the presence of mutant forms of hSel-10. (C, D) Plasmids encoding Flag-parkin, hSel-10, ubiquitin, UbcH7, or UbcH8, were co-transfected in the combinations indicated, and ubiquitinated species were detected as above.

FIG. 3 illustrates that parkin associates with Cul-1, but not with Skp1 or Rbx1. (A) HeLa cells were transiently transfected with expression constructs encoding Flag-parkin, HA-Cul-1 (86 kDa), and His₆-Skp1 (19 kDa), in the presence or absence of Myc-hSel-10 (69 kDa). Lysates were immunoprecipitated with anti-Flag antibodies, and probed by Western blotting, as indicated. (B) Left panel: Insect cells were co-infected with baculoviruses expressing GST-parkin, HA-Cul-1, His₆-Skp1, Rbx1 (11 kDa), and either Flag-hSel-10 (110 kDa) or Flag-β-TrCP. GST-parkin was pulled down with Sepharose-glutathione beads, and complexes were probed by Western blotting. Right panel: Insect cells were infected as above, with or without His₆-Skp1. Skp1-associated complexes were isolated from cell lysates by nickel-agarose pull-downs, and analyzed by Western blotting. (C) Homogenates of 1 g of frozen frontal cortex from an ARPD case or an age-matched control were immunoprecipitated with a monoclonal antibody specific for parkin, and probed, as indicated, by Western blotting. Western-blot analysis of parkin and hSel-10 is shown in FIG. 1E.

FIG. 4 demonstrates that cyclin E is a candidate substrate of the parkin/Cul-1/hSel-10 ubiquitin ligase complex. (A) Insect cells were co-infected with baculoviruses expressing Flag-hSel-10 (110 kDa) or Flag-β-TrCP, GST-parkin, HA-CDK2, and either His₆-cyclin E or His₆-cyclin A1 (50 kDa). Cyclin-E- or cyclin-A1-associated complexes were isolated from cell lysates by nickel-agarose pull-downs, and analyzed by Western blotting, as indicated. (B) Flag-parkin-associated complexes (immunoprecipitated from HeLa cells transfected with Flag-tagged wild-type or T240R mutant parkin) were incubated with recombinant His₆-cyclin E, HA-CDK2, E1, UbcH7, and ubiquitin, in the presence of an ATP-regenerating system. Recombinant His₆-cyclin E generated in insect cells appears as a 51-kDa band (arrow) and a minor contaminating species at 95-kDa. The asterisk indicates the position of an immunoglobulin heavy chain. (C) Parkin deficiency leads to cyclin E accumulation. Dissociated cortical neurons from E16.5 mice were cultured as described (see Examples), transfected with 25 nM parkin siRNA or control (DAT) siRNA, and then treated for 24 h with 500 μM kainate. After 48 h, cells were extracted with loading buffer, and lysates were probed, by Western blotting, for parkin, cyclin E, β-Actin, and cleaved PARP. The asterisk indicates a non-specific band; the arrow indicates the position of parkin (52 kDa). Densitometric analysis of protein bands (NIH Image 1.62) and relative band intensities are presented as the mean±SEM of three independent measurements. *=p<0.01, Student's t test (D) Homogenates of substantia nigra (SN) tissue from ARPD brain age-matched control, 2 sporadic PD cases, and 2 sporadic Alzheimer's disease (AD) cases were probed, by Western blotting, for parkin, cyclin E, UbcH7, or cyclin D1 (35 kDa).

FIG. 5 shows that parkin overexpression attenuates the accumulation of cyclin E in kainate-treated cells. Cerebellar granule cells from post-natal day 6 (P6) mice were transfected in suspension with a bicistronic expression plasmid for wild-type parkin (along with GFP) or with vector (GFP alone), cultured at a density of 75,000 cells/cm² for 72 h, and then treated with or without 500 μM kainate for 24 h. (A) Cells were extracted directly with loading buffer, and lysates were analyzed by Western blotting, as indicated. (B-M) Granule cell cultures were fixed, stained with a specific antibody against cyclin E, and then visualized by fluorescence microscopy for cyclin E (red) and GFP (green). Arrows indicate parkin-transfected neurons that display reduced accumulation of cyclin E relative to surrounding untransfected neurons (panels K, K′) or neurons transfected with vector (panels H, H′). scale bar=50 m

FIG. 6 illustrates that parkin protects post-mitotic neurons from kainate-mediated toxicity. Cerebellar granule cells from P6 mice were transfected as in FIG. 6, and then cultured in the presence or absence of kainate (500 μM) for 24 h. (A-L) Cells were stained for 20 min with 0.5 μg/ml Hoechst dye, and apoptotic nuclei were visualized by fluorescence microscopy. Arrows point to transfected apoptotic neurons apparent in the vector-only transfected cultures (panel K) but not the parkin-transfected culture (panel L). scale bar=100 μm (M) Cell protection in the absence or presence of kainate is expressed as a percentage of GFP-positive (transfected) cells that are also Hoechst-positive (apoptotic). Data are shown as the mean±SEM for 2 independent experiments performed in triplicate. Statistical significance was assessed using one-way ANOVA with Tukey-Kramer post-hoc tests between each group. *=p<0.005

FIG. 7 shows that parkin deficiency potentiates kainate-mediated toxicity in midbrain dopamine neurons. Dissociated midbrain cultures from E13.5 mice were prepared as described (see Examples), transfected with 25 nM parkin or control (SERT) siRNA, and treated for 24 h with 250 μM kainate (A-X) or 1 μM MPP⁺ (U-X). Cells were treated with Hoechst dye, fixed, and stained with rabbit polyclonal antibodies against either mouse parkin or cyclin E (green) and a rat monoclonal antibody against DAT (red). Immunostaining and apoptotic nuclei were visualized by fluorescence microscopy. Arrows point to examples of DAT-positive, cyclin-E-positive neurons (S) and DAT-positive neurons with apoptotic (Hoechst-positive) nuclei (I, J, and T). Total DAT-specific immunoreactivity (pixels), across 9 fields of view at 20×, was quantified in triplicate using Image software (Scion). Cytoplasmic parkin and cyclin E immunoreactivity (mean pixel density), in DAT-positive neurons, were similarly quantified. Data are shown as the mean±SEM. Statistical significance was assessed using one-way ANOVA with Tukey-Kramer post-hoc tests between each group. *=p<0.01; scale bar=150 μm

FIG. 8 demonstrates that parkin overexpression protects midbrain dopamine neurons from kainate-mediated toxicity. Primary E13.5 midbrain cultures were prepared as above, infected with human parkin or control (GFP) lentiviral vectors, and subsequently cultured for 24 h with (G-L) or without (A-F) 250 μM kainate. Cultures were subsequently fixed and stained with a monoclonal antibody specific for human parkin (which is not cross-reactive with the endogenous mouse parkin; FIGS. 9-12) and a rat monoclonal antibody against DAT. The arrow points to an example of a GFP-infected, kainate-treated, DAT-positive neuron with diminished DAT immunoreactivity. Total DAT-specific immunoreactivity (pixels), across 9 fields of view at 20×, was quantified in triplicate, as in FIG. 7. Data are shown as the mean±SEM. Statistical significance was assessed using one-way ANOVA with Tukey-Kramer post-hoc tests between each group. *=p<0.01; scale bar=1501 m (M) Furthermore, parkin overexpression did not alter DAT immunoreactivity in primary midbrain neuron cultures in the absence of toxin.

FIG. 9 sets forth additional Western-blot analyses. (A) Monoclonal antibody 2E10 recognizes the amino-terminal UHD of human parkin. HeLa cells were transfected with wild-type or a UHD-deletion form of parkin, and cell lysates were probed by Western blotting with 2E10 or a polyclonal antibody that recognizes the carboxyl-terminus of parkin. A 52-kDa species is recognized by both antibodies; in contrast. the 42-kDa polypeptide appears to represent a processed form of parkin, and is recognized by the polyclonal antibody (Schlossmacher et al., Parkin localizes to the Lewy bodies of Parkinson disease and dementia with Lewy bodies. Am. J. Pathol., 160:1655-67, 2002) (data not shown). (B) Full-length parkin interacts with full-length and deletion forms of hSel-10. HeLa cells were transiently transfected with expression vectors encoding Flag-tagged parkin, or Myc-tagged wild-type or Myc-tagged deletion forms of h-Sel-10. Anti-Flag immunoprecipitates were analyzed by Western blotting, as indicated. (C) The T240R mutation of parkin attenuates the interaction between parkin and Cul-1. HeLa cells were transiently transfected with expression vectors encoding Flag-tagged wild-type or T240R ARPD mutant forms of parkin, along with tagged forms of hSel-10 and Cul-1. Anti-Flag immunoprecipitates were analyzed by Western blotting as indicated. (D) HSel-10 interacts with both parkin and SCF complex components. Insect cells were co-infected with baculoviruses expressing GST-parkin, HA-Cul-1, His₆-Skp1, and Rbx1, with or without Flag-hSel-10 (110 kDa). Anti-Flag immunoprecipitates were analyzed by Western blotting, as indicated.

FIG. 10 further illustrates parkin/cyclin E interaction. (A) Altered parkin expression does not affect cyclin E mRNA levels. Total RNA was extracted from granule cell cultures transfected with parkin or vector alone (FIG. 5), and from frontal cortex tissue from parkin-deficient ARPD (or age-matched control; FIG. 4). Cyclin E and β-actin mRNA levels were determined by quantitative RT-PCR, as described (Troy et al., Death in the balance: alternative participation of the caspase-2 and -9 pathways in neuronal death induced by nerve growth factor deprivation. J. Neurosci., 21:5007-16, 2001). (B-D) Both cyclin E immunoreactivity (p<0.05) and apoptosis (p<0.05) are increased in DAT-negative neurons of primary midbrain cultures treated with parkin siRNA (and kainate) relative to control siRNA (and kainate). However, the increased cyclin E immunoreactivity and apoptosis are both less marked than in DAT-positive neurons (p<0.05 for both measures). DAT-negative neurons in midbrain cultures were analyzed as in FIG. 7; DAT-positive neuron data is from FIG. 7. Cytoplasmic parkin and cyclin E immunoreactivity (mean pixel density) in DAT-negative neurons were quantified in triplicate, across 9 fields of view at 20×. Data are shown as the mean±SEM. Statistical significance was assessed using one-way ANOVA with Tukey-Kramer post-hoc tests between each group. *=p<0.01; **=p<0.05; scale bar=150 μm (E-J) Human parkin lentiviral vectors efficiently infect cultured murine midbrain dopamine neurons. E13.5 murine midbrain cultures were infected with lentiviruses encoding human parkin or control (GFP), as described in FIG. 8. Fixed cells were immunostained with the human parkin-specific monoclonal antibody, 2E10 (which does not cross-react with endogenous murine parkin; panels F and I), and a rat antibody against the dopamine transporter (panel G). scale bar=150 μm

FIG. 11 demonstrates that parkin overexpression does not appear to protect dopamine neurons from MPP⁺. (A-F) Murine midbrain cultures were infected with lentiviruses encoding GFP or human parkin, as described in FIG. 8, treated for 24 h with 10 μM MPP⁺, and immunostained with the human parkin antibody (red) and a DAT-specific antibody (green). scale bar=150 μm (G) DAT immunoreactivity was measured and analyzed statistically, as described in FIG. 7.

FIG. 12 sets forth results of analyses using frontal cortex. (A) Frontal cortex extracts from three additional ARPD cases and three additional age-matched controls were prepared as described in FIG. 4D, and analyzed by Western blotting for cyclin E and UbcH7. (B) Cyclin E is variably elevated in extracts of frontal cortex from sporadic AD and PD patients. Frontal cortex extracts from parkin-deficient ARPD cases and age-matched controls (as in A), Huntington's disease (HD) cases, sporadic Parkinson's disease (PD), and sporadic Alzheimer's disease (AD) were prepared as described in FIG. 4D, and analyzed by Western blotting for cyclin E and UbcH7. (C) Most DAT-negative cells in E14 primary midbrain cultures are GABAergic. Embryonic midbrain cultures, as described in FIG. 7, were stained for DAT (red) or GAD-65 (green). scale bar=50 μm

FIG. 13 sets forth the amino acid sequence of parkin protein.

FIG. 14 shows that DJ-1-deficient ES cells are sensitized to oxidative Stress. (A) Schematic map of the murine DJ-1 gene in clone F063A04. The retroviral insertion places the engrailed-2 (En2) intron, the splice acceptor (SA), and the β-galactosidase/neomycin resistance gene fusion (β-geo) between exons 6 and 7. (B) Southern blot analysis of KpnI-digested genomic DNA from DJ-1 homozygous mutant (−/−), WT (+/+), and heterozygous (+/−)cells, probed with murine DJ-1 cDNA. WT DNA shows a predicted 14-kb band (WT), whereas the mutant allele migrates as a 9-kb band (insertion). (C) Western blot (WB) of ES cell lysates from WT (+/+), DJ-1 heterozygous (+/−), and mutant homozygous (−/−) clones with antibodies to murine DJ-1 (α-DJ-1) or β-actin (α-β-actin). DJ-1 migrates at 20 kDa, β-actin at 45 kDa. (D) ES cells were exposed to 0, 5, 10, and 20 μM H2O2 for 15 h and viability was assayed by MTT. Responses of DJ-1 heterozygous cells (diamonds) and DJ-1 knockout clones 9 (open circles), 16 (solid circles), 23 (squares), and 32 (triangles) are shown. **p≦0.01; ***p≦0.0001. (E and F) Cell death of DJ-1 heterozygous and DJ-1-deficient cells (clone 32) after exposure to H2O2 (10 μM) was quantified by staining with PI and an antibody to AV with subsequent FACS analysis. AV staining marks cells undergoing apoptosis, whereas PI staining indicates dead cells. *p≦0.05. (G) DJ-1 heterozygous (+/−) and knockout (clone 32; −/−) cells were assayed at 1, 6, and 24 h after treatment with 10 μM H2O2 by Western blotting for cleaved PARP (89 kDa), which indicates apoptosis. No band is seen for cleaved PARP or β-actin for the DJ-1-deficient cells at 24 h due to cell death. Data represent means±standard error of the mean (SEM) and were analyzed by ANOVA with Fisher's post-hoc test.

FIG. 15 depicts specificity and mechanism of altered toxin sensitivity in DJ-1-deficient cells. (A-C) Cell viability of DJ-1 heterozygous cells (solid bar) and DJ-1-deficient knockout clone 32 cells (open bar) after 15 h exposure to H2O2 (A), lactacystin (B), or tunicamycin (C) as assayed by MTT reduction. *p≦0.05. (D) DJ-1-deficient knockout cells (clone 32) were transiently transfected with plasmids containing WT human DJ-1 vector (solid bar) and PD-associated L166P mutant DJ-1 vector (gray bar); as a control, knockout cells were also transfected with vector alone (open bar). 48 h after transfection, cells were exposed to 10 μM H2O2 for 15 h and then assayed by MTT reduction. WT human DJ-1 significantly enhanced survival of the knockout cells, whereas the L166P mutant did not. Similar results were obtained at 20 μM H2O2 and with a second DJ-1-deficient clone (unpublished data). Transfection efficiency exceeded 90% in all cases and protein expression level was comparable for human WT and L166P mutant DJ-1 as determined by Western blotting (FIG. S1). *p≦0.05. (E) DJ-1-deficient cells (clone 32; open bar) and control heterozygous cells (solid bar) were assayed for intracellular formation of ROS in response to H2O2 treatment (15 min, 1 or 10 μM) using DHR and FACS analysis. (F) Protein carbonyl levels were measured by spectrophotometric analysis of DNP-conjugated lysates from DJ-1-deficient (clone 32; solid red line) and control heterozygous cells (dashed blue line). Data are shown as the mean±SEM and were analyzed by ANOVA with Fisher's post-hoc test.

FIG. 16 demonstrates that DJ-1-Deficient ES Cell Cultures Display Reduced DN Production. (A) The SDIA coculture method. DJ-1 knockout or control heterozygous ES cells are cocultured with mouse stromal cells (MS5) in the absence of serum and leukemia inhibitory factor for 18 DIV. (B) DN production was quantified at 18 DIV by 3H-dopamine uptake assay. DJ-1-deficient ES cell cultures were defective relative to heterozygous control cultures. (C-D) Neuron production was quantified by immunohistochemical analysis as a percent of TuJ1-positive colonies that express TH (C) or GABA (D). Quantification of TH and GABA immunostaining was performed on all colonies in each of three independent wells. Colonies were scored as positive if any immunostained cells were present. *p≦0.05. (E) The absolute number of TuJ1-positive colonies was not significantly different between the two genotypes. (F) Kinetic analysis of DN differentiation in DJ-1-deficient cultures (clone 32, solid square) and heterozygous controls (open circle) as quantified by 3H-dopamine uptake assay. *p≦0.05. (G) DJ-1-deficient (open bar) and heterozygous control (closed bar) cultures differentiated for 9 DIV and then exposed to 6-OHDA at the indicated dose for 72 h. DNs were quantified by 3H-dopamine uptake assay. Data represent the means±SEM and were analyzed by ANOVA followed by Fisher's post-hoc test. *p≦0.05.

FIG. 17 shows neuronal differentiation of DJ-1-deficient and control heterozygous ES cell cultures. (A-L) DJ-1 heterozygous (+/−; A-F) and knockout (−/−[clone 32]; G-L) cultures were differentiated by SDIA for 18 DIV and immunostained with antibodies to TH (green) and TuJ1 (red). Images of both (Merge) are also shown. (A′-L′) Immunostaining of DJ-1 heterozygous (+/−, A′-F′) and deficient (−/−, G′-L′) cultures with antibodies for GABA (green) and TuJ1 (red). Scale bar, 50 μm. Images of both (Merge) are also shown.

FIG. 18 shows RNAi “Knockdown” of DJ-1 in Primary Embryonic Midbrain DNs Display Increased Sensitivity to Oxidative Stress. (A-P) Primary midbrain cultures from E13.5 embryos were infected with lentiviral vectors encoding DJ-1 shRNA (or vector alone) under the regulation of the control vector (A-H) or the U6 promoter (I-P). Cells were cultured for 1 wk after infection and then exposed to H2O2 (5 μM; E-H and M-P) for 24 h. Cultures were immunostained for TH (B, F, J, and N) or DAT (C, G, K, or O) and visualized by confocal microscopy. Images containing all stains are included (Merge; D, H, L, and P). Scale bar, 100 μm. (Q) Cell lysates prepared from midbrain primary cultures infected with DJ-1 shRNA lentivirus (or control vector) were analyzed by Western blotting for murine DJ-1 or β-actin. (R-T) Quantification of TH, DAT, and GFP signal was performed on ten randomly selected fields in each of three wells for each condition. Red triangles, DJ-1 shRNA treated; black circles, control vector. Data represent the means±SEM and were analyzed by ANOVA followed by Fisher's post-hoc test. *p≦0.05.

FIG. 19 sets forth Quantitative Real-Time PCR for DJ-1 Gene Expression. (A) Real-time PCR analyses of DJ-1 cDNA in WT (+/+), heterozygous (+/−), and knockout (−/−) cultures. Each expression value was normalized to that of α-actin and expressed relative to the respective value of the WT (+/+) control. These gene expression patterns were replicated in at least three independent PCR experiments. Total RNA from ES cells differentiated with the SDIA method for 18 days was isolated using the Absolutely RNA Miniprep kit (Stratagene, La Jolla, Calif., United States). Synthesis of cDNA was performed using the SuperScript first strand synthesis system for RT-PCR (Invitrogen). Real-time PCR reactions were optimized to determine the linear amplification range. Quantitative real-time RT-PCRs were performed (Stratagene MX3000P) using the QuantiTect SYBR Green PCR Master Mix (Qiagen, Valencia, Calif., United States) according to the manufacturer's instructions. DJ-1 primer sequences were 5′-CGAAGAAATTCGATGGCTTCCAAAAGAGCTCTGGT-3′ and 5′-CAGACTCGAGCTGCTTCACATACTACTGCTGAGGT-3′; primers used for β-actin were 5′-TTTTGGATGCAAGGTCACAA-3′ and 5′-CTCCACAATGGCTAGTGCAA-3′. For quantitative analyses, PCR product levels were measured in real time during the annealing step, and values were normalized to those of β-actin. (B) Ethidium bromide staining of the PCR products obtained after 29 cycles for DJ-1 (625 bp) and β-actin (350 bp).

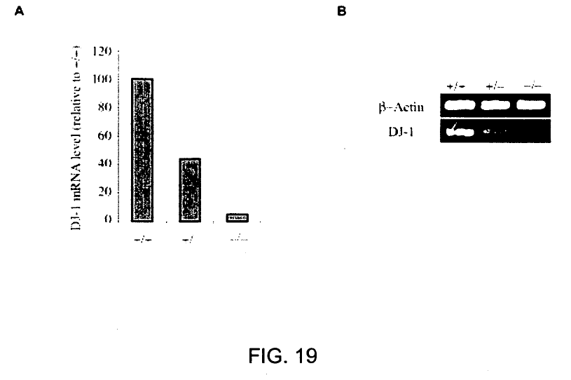

FIG. 20 depicts an analysis of DJ-1-Deficient ES Cells. (A and B) Cell viability of DJ-1 heterozygous cells (solid bar) and DJ-1-deficient knockout clone 32 (open bar) after exposure to CuCl2 or staurosporine at the doses indicated. (C) MTT values of untreated DJ-1-deficient ES cell clones and the control heterozygous cells. Assays were performed exactly as in FIG. 15, but in the absence of toxin. (D) MTT values of untreated DJ-1-deficient ES cells transfected with vector alone or various DJ-1-encoding plasmids. Transfection and expression of WT DJ-1 or mutant forms of DJ-1 does not alter the basal metabolic activity or viability of the cells. (E) Western blotting of extracts from ES cells transfected with vectors harboring WT human DJ-1 or the L166P mutant.

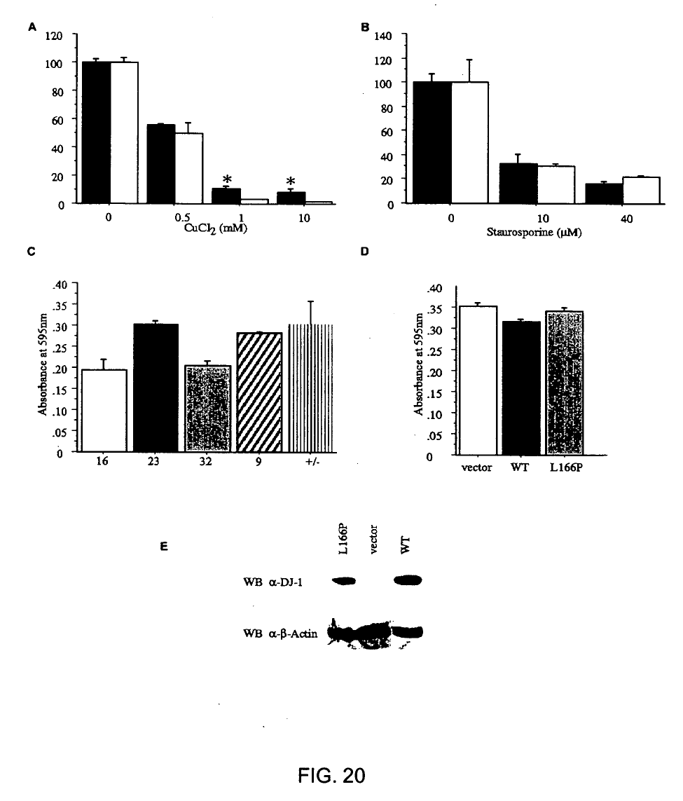

FIG. 21 further illustrates Immunocytochemistry for HB9 and GABA Neurons in DJ-1-Deficient and Control Heterozygous ES Cells. Both cell cultures were differentiated by SDIA for 18 DIV. Cells were fixed with 4% paraformaldehyde and stained with mouse monoclonal antibodies against HB9 (gift from T. Jessell, dilution 1:50) and rabbit polyclonal antibodies against GABA (Sigma, dilution 1:1000) as in FIG. 5. Scale bar, 50 μM.

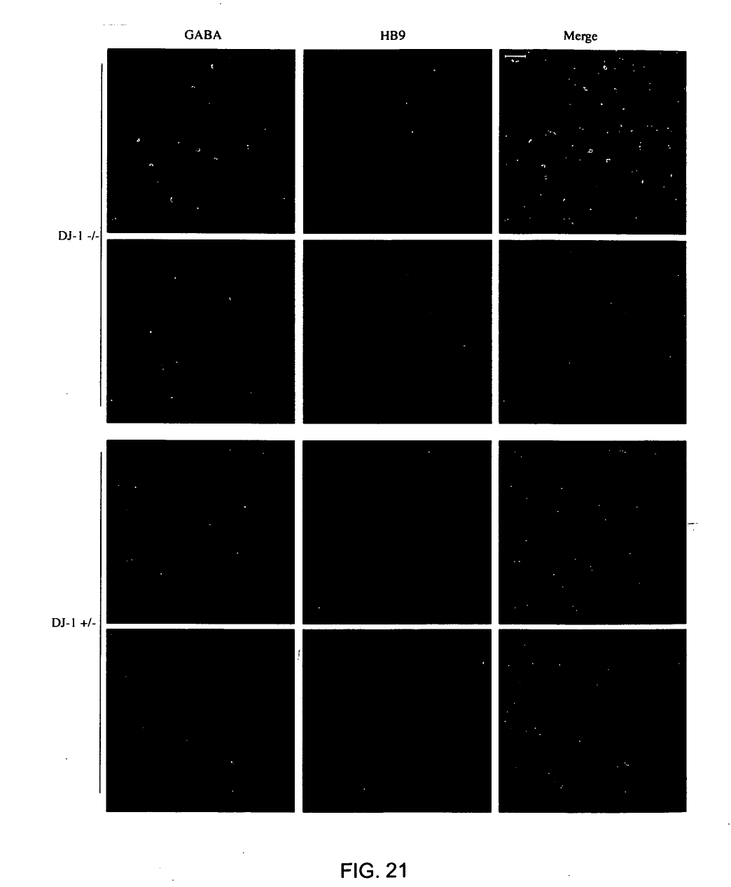

FIG. 22 illustrates that DJ-1 is a redox-dependent molecular chaperone. (A) Aggregation of CS was monitored at 43° C. after addition of either 0.8 μM CS alone (black), or along with 8.0 μM RNase A (purple), 0.5 μM DJ-1 (aqua), 2.0 μM DJ-1 (blue), 4.0 μM DJ-1 (red), or 2.0 μM Hsp27 (green). (B) Aggregation of 0.8 μM CS after 30 min at 4° C. (unfilled bar) is inhibited by 4.0 μM WT DJ-1 (black bar) but not 4.0 μM L166P mutant DJ-1 (gray bar). Data are shown as the mean±SEM and were analyzed by ANOVA with Fisher's post-hoc test. *p<0.05. (C) Aggregation of insulin (26 μM) B chains induced by 20 mM DTT at 25° C. Insulin alone (black) or in the presence of 4.0 μM RNase A (purple), 0.5 μM DJ-1 (aqua), 2.0 μM DJ-1 (blue), 4.0 μM DJ-1 (red), or 2.0 μM Hsp27 (green). (D) CS thermal aggregation (unfilled bar) is suppressed by 4 μM DJ-1 (black bar), but chaperone activity is abrogated upon incubation of DJ-1 with 0.5 mM DTT for 10 min at 4° C. (gray bar). Further treatment of DTT-reduced DJ-1 with 10 mM H2O2 for 10 min at 4° C. leads to reactivation of CS suppression (hatched bar). Data are shown as the mean±SEM and were analyzed by ANOVA with Fisher's post-hoc test. *p<0.05.

FIG. 23 shows that DJ-1 inhibits formation of αSyn protofibrils and fibrils in vitro. (A) Purified αSyn (200 μM) was incubated for 2 h at 55° C. in the presence of WT DJ-1, L166P mutant DJ-1, GST, or Hsp27 (all at 100 μM). WT DJ-1 inhibits accumulation of αSyn protofibrils in vitro, while L166P mutant DJ-1, GST, and Hsp27 do not. (B) Suppression of αSyn protofibril formation by WT DJ-1 (in triplicate) was quantified as compared to GST (as a negative control) and mutant L166P DJ-1. Data are shown as the mean±SEM and were analyzed by ANOVA with Fisher's post-hoc test. *p<0.05. (C) Purified αSyn (200 μM) was incubated for 1 wk at 37° C. in the presence of WT DJ-1, L166P mutant DJ-1, or GST (all at 100 μM). WT DJ-1 inhibits formation of mature Congo red-positive αSyn fibrils. Data are shown as the mean±SEM and were analyzed by ANOVA with Fisher's post-hoc test. *p<0.05.

FIG. 24 illustrates that overexpression of WT DJ-1 inhibits aggregation of αSyn in vivo. (A) CAD murine neuroblastoma cells were transfected with Flag-αSyn along with WT DJ-1, L166P clinical mutant, or vector alone, and were differentiated in vitro via serum withdrawal. Cells were subsequently treated with 2 mM FeCl2 (Fe), 5 μM lactacystin (LC), or media alone (0). Triton X-100-soluble (Tx-100 sol) and Triton X-100-insoluble (Tx-100 insol) fractions were analyzed by Western blotting. Upon FeCl2 treatment, αSyn accumulates in the Triton X-100-insoluble fraction, and accumulation of insoluble αSyn is inhibited by overexpression of WT DJ-1 (left) but not the L166P clinical mutant (right). (B) Triton X-100-insoluble αSyn as quantified by NIH Image J of a Western blot (from [A]). (C) Heterozygous (+/−) and DJ-1 deficient (−/−) ES cells were differentiated using the embryoid body protocol. Cells were transfected with Flag-αSyn (F-αSyn), and, after 48 h, treated with 2 mM FeCl2 or with media alone for 18 h. Cell lysates were analyzed by Western blotting for αSyn or β-actin. In the Triton X-100-soluble fraction (Tx-100 sol), DJ-1 accumulated to a similar extent in the knockout and control cells. In contrast, αSyn accumulation in the insoluble pool (Tx-100 insol) was detectable only in the knockout cells, and this was further promoted by FeCl2 treatment. (D) CAD cells transfected with Flag-αSyn (F-αSyn) along with WT DJ-1 (or vector alone) were treated with 2 mM FeCl2 or media alone for 18 h. Triton X-100-soluble cell lysates were immunoprecipitated with a mouse monoclonal antibody for the Flag epitope and Western blotted for DJ-1. FeCl2 treatment induces association of Flag-αSyn with WT DJ-1. Lysates represent 20% input of the immunoprecipitation (IP α-Flag). The Triton X-100 soluble pool of DJ-1 is reduced by αSyn overexpression (but not vector control), particularly in the context of FeCl2 treatment (bottom). (E) DJ-1 colocalizes with αSyn in the Triton X-100-insoluble fraction upon FeCl2 treatment. The Western blot from (A) was stripped and reprobed for DJ-1.

FIG. 25 shows that DJ-1 Inhibits Formation of αSyn Intracytoplasmic Inclusions. (A-L) CAD murine neuroblastoma cells were transfected with WT DJ-1 (A-F), L166P DJ-1 (G-I) or vector control (J-L), along with Flag-αSyn (D-L) or vector control (A-C) and differentiated in vitro by serum withdrawal for 72 h. Cells were fixed and stained with a mouse monoclonal antibody for αSyn and ToPro3, a nuclear dye, and images were obtained by confocal microscopy. Transfection of Flag-αSyn induced formation of intracytoplasmic inclusions (arrows). Scale bar, 20 μm. (M) Quantification of cells with inclusions was performed on ten random images from each of three wells per condition. Images were quantified by an observer blinded to the experiment. A significantly lower percentage of cells harbor inclusions in the context of WT DJ-1 overexpression. Aggregation is expressed as the percentage of cells containing αSyn aggregates per frame. Total cell number per frame, as determined by ToPro3 staining, did not differ significantly (FIG. 30). Data are shown as the mean±SEM, and were analyzed by ANOVA with Fisher's post-hoc test. *p<0.05. (N-S) Cells were fixed and stained with a monoclonal antibody for αSyn and a polyclonal antibody that recognizes both transfected human DJ-1 and endogenous murine DJ-1. DJ-1 does not appear to colocalize with the αSyn aggregates. Scale bar, 20 μm.

FIG. 26 shows that DJ-1 inhibits formation of NFL intracytoplasmic inclusions. (A-L) CAD cells were transfected with an aggregation-prone mutant NFL (Q333P) plasmid, as well as WT human DJ-1 plasmid (that also harbors GFP; E-H), L166P mutant DJ-1 (that also harbors GFP; I-L), or control GFP vector (A-D). After 72 h in culture, cells were fixed and stained with a mouse monoclonal antibody for NFL and ToPro3, a nuclear dye. Scale bar, 100 μm. (M-R) CAD cell transfectants, as above, were fixed and stained with a polyclonal antibody for NFL (Perez-Olle et al. 2002) along with a mouse monoclonal antibody specific for the transfected human DJ-1. Scale bar, 20 μm. (S) Quantification of CAD cell NFL aggregates was performed using confocal microscopy. Images from ten randomly selected fields in each of three wells were quantified for the presence of aggregates for each condition and presented as a percentage of total cells per field. Total cell number was determined by ToPro3 nuclear staining and did not differ significantly (FIG. 30). Data are shown as the mean±SEM and were analyzed by ANOVA with Fisher's post-hoc test. *p<0.05.

FIG. 27 shows that DJ-1 in vitro chaperone activity and in vivo oxidative stress protection activity requires cysteine 53 but not cysteine 106. (A) DJ-1 cysteine-to-alanine mutants C106A, C53A, and a triple mutant that harbors mutations at all three cysteines in DJ-1 (C106A/C53A/C46A), as well as L166P, were tested for in vitro chaperone activity by CS aggregation suppression assay. (B) Self-association of DJ-1 cysteine mutants. Murine neuroblastoma CAD cells were transiently cotransfected with Flag-tagged human DJ-1 vectors (either WT or mutant) along with WT YFP-tagged human DJ-1. Lysates were immunoprecipitated with anti-Flag antibodies and probed by Western blotting with an antibody specific for human DJ-1. WT Flag-DJ-1, C106A DJ-1, C53A DJ-1, and C106A/C53A/C46A DJ-1 effectively coprecipitated WT GFP-DJ-1, whereas the L166P mutant Flag-DJ-1 failed to do so. Lysates represent 20% of the input for the immunoprecipitate; Flag-DJ-1 migrates at 22 kDa, and YFP-DJ-1 migrates at 50 kDa. (C) DJ-1-deficient ES cells were transiently transfected with vector alone, WT DJ-1, or DJ-1 cysteine mutants, and exposed to 10 μM H2O2 for 15 h followed by MTT assay. The viability of the cells in the absence of drug treatment was not altered by the expression of WT or mutant DJ-1). Data are shown as the mean±SEM and were analyzed by ANOVA with Fisher's post-hoc test. *p<0.05. (D) Expression levels of WT and mutant forms of DJ-1 were comparable as determined by Western blotting for human DJ-1 and β-actin.

FIG. 28 depicts additional structural and functional analyses of DJ-1 in vitro. (A) DJ-1 catalase activity was quantified as compared to catalase I (5 μg/ml). DJ-1 does not display catalase activity even at concentrations as high as 5 mg/ml. (B) Addition of DJ-1 at 5 mg/ml does not alter catalase activity of the catalase I-positive control, indicating that there are no inhibitory elements present in the DJ-1 preparation. (C) Purity of bacterially produced DJ-1 utilized in the in vitro assays was assessed to be >99% by SDS-PAGE and colloidal Coomassie staining. (D) GST thermal aggregation (0.4 μM, black circles) is suppressed by WT DJ-1 (2 μM, red squares) and by positive control Hsp27 (2 μM, green stars), but not by L166P mutant DJ-1 (2 μM, blue triangles) or by RNase A (2 μM, purple diamonds). (E) Far-ultraviolet CD spectra of WT DJ-1 (blue triangles) and the L166P mutant (red squares); mean residue ellipticity (Θ) equals ° C.·cm2·dmol-1. The mutant protein displays significantly reduced secondary structure. CD spectra of DJ-1 (40 μM in 10 mM PBS [pH 7.4]) were recorded on an Aviv 62A sCD spectrometer at 4° C. in a 0.02-cm path length cuvette, and α-helix and β-sheet content were estimated as described (Sreerama and Woody 2003). Based on an initial evaluation of the spectra, the WT spectrum was analyzed using a basis set appropriate for folded proteins, whereas the mutant spectrum was analyzed using a basis set suited for unstructured proteins. Thermal stability was determined by monitoring the change in mean residue ellipticity ([Θ], equal to ° C.·cm2·dmol−1) at 222 nm as a function of temperature. Thermal melts were performed in 4° C. increments with an equilibration time of 1 min and an integration time of 30 sec, using a 0.1-cm path length cuvette. (F) Thermal denaturation curves for WT and mutant L166P DJ-1; mean residue ellipticity (Θ)222 is equal to °C.·cm2·dmol−1 at 222 nm. (G) Redox regulation is unaffected by the C106A mutation. Redox regulation of C106A DJ-1 was assayed via DTT inactivation (0.5 mM) in the CS aggregation suppression assay. (H) Protofibril preparations (as in FIGS. 23A and 23B, incubated for 2 h at 55° C.) do not contain Congo red-positive mature fibrils. Untreated αSyn preparations (open bars) and protofibril preparations (filled bars) were subjected to Congo red analysis as in FIG. 23C.

FIG. 29 shows additional studies of DJ-1 chaperone activity in vivo. (A) Undifferentiated ES cells were transfected with Flag-αSyn and treated with 2 mM FeCl2 (Fe) or media alone (O) as described in FIG. 24. As expected, undifferentiated ES cultures do not express endogenous αSyn. Furthermore, the transfected Flag-αSyn does not accumulate in the Triton X-100-insoluble fraction of undifferentiated cells, in contrast to differentiated cultures. (B) Overexpression of WT DJ-1 does not significantly alter the half-life of soluble Flag-αSyn. CAD murine neuroblastoma cells were stably transfected with Flag-tagged human α-synuclein using standard techniques. 2×105 cells in a 24-well format were transiently transfected with eukaryotic expression constructs encoding WT human DJ-1 or empty vector. After 36 h, cells were starved for 1 h with DMEM lacking cysteine and methionine and supplemented with 8% dialyzed FBS. Cells were pulsed for 2 h with 10 μCi[35S]-L-Met/L-Cys (EasyTides; Perkin Elmer, Wellesley, Calif., United States) per well, washed twice, and chased at the indicated intervals with complete medium. Flag-αSyn was immunoprecipitated with Flag antibody-conjugated agarose beads (Sigma), subjected to SDS-PAGE, and visualized by autoradiography. (C) Flag-αSyn from (B) was quantitated using NIH Image J.

FIG. 30 depicts the additional studies of DJ-1 mutations. (A) Overexpression of WT DJ-1 or L166P DJ-1 in the context of αSyn aggregation does not alter cell number. Cells from FIG. 4M were quantified via ToPro3 nuclear staining and are expressed as number of cells per field from ten independent fields in each of three wells. Data are shown as the mean±SEM and were analyzed by ANOVA with Fisher's post-hoc test. *p<0. (B) Overexpression of WT DJ-1 or L166P mutant DJ-1 in the context of Q333P mutant NFL aggregation does not alter cell number. GFP positive transfected cells from FIG. 5A-5L were quantified and are expressed as number of transfected cells per field from ten independent fields in each of three wells. Data are shown as the mean±SEM and were analyzed by ANOVA with Fisher's post-hoc test. *p<0. (C) Overexpression of WT DJ-1, but not L166P mutant DJ-1, rescues cells from Q333P mutant NFL toxicity. HeLa cells were transfected with Q333P mutant NFL along with WT human DJ-1, L166P mutant DJ-1, or vector control. After 72 h, cells were assayed by MTT reduction assay (which detects reduction of 3-(4,5-dimethylthiazol-2-yl)-2,5-diphenyltetrazolium bromide by metabolic enzymes) (Martinat et al. 2004). Data are shown as the mean±SEM and were analyzed by ANOVA with Fisher's post-hoc test. *p<0. (D) C53A mutant DJ-1 is unable to rescue cells from Q333P mutant NFL toxicity. Undifferentiated ES cells were transfected with Q333P mutant NFL along with WT human DJ-1, C53A mutant DJ-1, or vector control. After 72 h, cells were assayed by MTT reduction assay (Martinat et al. 2004). Data are shown as the mean±SEM and were analyzed by ANOVA with Fisher's post-hoc test. *p<0. (E) Coexpression of DJ-1 with NFL does not alter NFL expression levels. CAD cells were transfected with Q333P mutant NFL and vector, WT DJ-1, C53A mutant DJ-1, or L166P mutant DJ-1. Cells were differentiated for 72 h and lysed to produce Triton X-100-soluble and -insoluble fractions. Lysates were exposed to Western blotting with an antibody against transfected human NFL. NFL is present only in the insoluble fraction, and expression of WT or mutant DJ-1 does not alter NFL expression levels.

FIG. 31 demonstrates DJ-1 localization does not appear altered by FeCl2 Treatment. CAD cells were transfected with WT DJ-1 and differentiated by serum withdrawal for 72 h. Cells were treated with medium alone (A-F) or medium with 2 mM FeCl2 (G-L) for 18 h prior to fixation with PFA. Cells were immunostained with rabbit anti-DJ-1 as described, followed by donkey anti-rabbit Cy5 (A, D, G, and J). Nuclei (B, E, H, and K) were visualized by incubation with the nuclear stain ToPro3 prior to imaging.

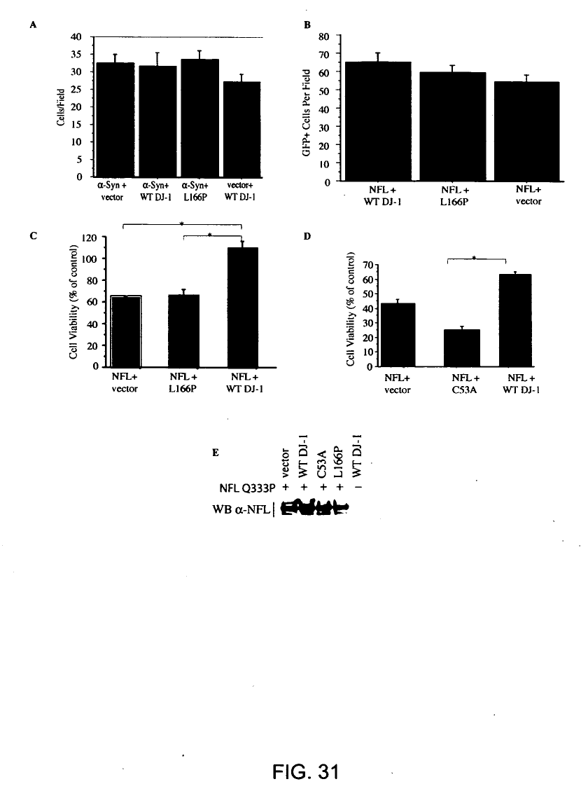

FIG. 32 shows that DJ-1 lacks protease and antioxidant activities.

FIG. 33 illustrates Embryonic Dopamine Neuron Specification. An early transcription factor network (Pax2, Pax5, Otx2) defines the midbrain-hindbrain boundary. Subsequently secreted factors including Sonic Hedgehog (SHH) and FGF8 define the ventral location of midbrain dopamine neuron precursors. F, forebrain; M, midbrain; H, hindbrain. Fp, floorplate; is, isthmus.

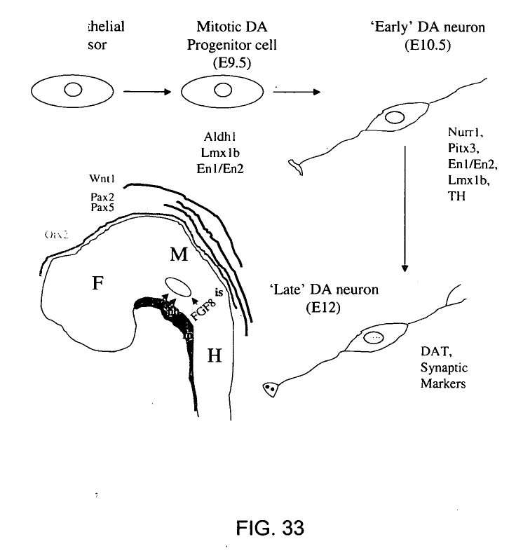

FIG. 34 shows that both intrinsic factor (left panel) and extrinsic factor (right panel) networks are thought to specify midbrain dopamine neurons.

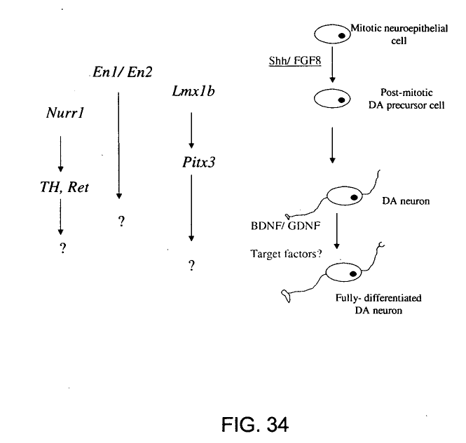

FIG. 35 depicts the generation of ‘marked’ mature midbrain dopamine neurons.

FIG. 36 shows that DY-1×Rosa26-lox-stop-lox-LacZ mice display specific marker expression in the substantia nigra but not elsewhere in the CNS. LacZ (blue) and TH (brown) double staining of SN sections. Control single transgenic Rosa26 mice (A) and DY1xLacZ double transgenic animals (B, C). The SN is outlined in (C).

FIG. 37 demonstrates stromal cell derived activity-mediated differentiation of DY-1 ES cells. DAT immunoreactivity (red) and YFP fluorescence (green) are shown. Most YFP-positive cells display eYFP expression.

FIG. 38 depicts embryoid body (EB) differentiation of DY-1 ES cells. LIF, leukemia inhibiting factor; ITSF, media supplemented with insulin, transferrin, selinium; bFGF, basic fibroblast growth factor; AA, ascorbic acid; div, days in vitro.

FIG. 39 shows dopamine uptake activity. (A) Time course of differentiation of DY-1 ES cells with SDIA as measured by dopamine uptake. (B) Dopamine uptake activity of DY-1 cultures transduced with lentiviral vectors at day 12 of SDIA differentiation.

FIG. 40 illustrates real-time Quantitative rt-PCR Analysis of Dopamine Neuron Development. (A-B) Real-time PCR analyses for genes specific to midbrain development. Each gene expression value was normalized to that of β-actin and expressed relative to the respective value of the stage 6 DIV GFP control-ES culture.

FIG. 41 illustrates replication-defective lentiviral vectors. (A) Single- and (B) two gene-vectors were assembled. LTR, viral long terminal repeat; cPPT, central polypurine tract; CTS, central terminal sequence; EF1α, EF1αpromoter region.

FIG. 42 depicts lentiviral transduction of PitX3, Nurr1, and other transcription factors modifies SDIA differentiation of DY-1 ES cells into DNs. DN differentiation was quantified by eYFP (A) fluorescence or TH immunoreactivity (B). Differentiation of serotonergic (C) and GABAergic (C) neurons was also quantified. All results were analyzed by ANOVA. Data represent the mean±SEM. The level of significance is indicated where *p≦0.05 and **p≦0.005.

FIG. 43 demonstrates the effect of different soluble factors on the differentiation of DY-1 into dopamine neurons with the EB method. (A) eYFP fluorescence or (B) DAT immunoreactivity was quantified by fluorescent confocal microscopy, see text for details. Data represent the mean±SEM. All results were analyzed by ANOVA. The level of significance is indicated where *p≦0.05 and **p≦0.005.

FIG. 44 shows embryoid body differentiation of DY-1 cells to mDNs. (A) EB differentiation of DY-1 cultures in vitro gives rise to DNs that co-express eYFP and DAT. Bar scale, 50 μm. (B) General scheme of the EB protocol and the expression profiles for Nurr1, PitX3, TH and DAT over the time course of the EB protocol, as analyzed by quantitative real-time rtPCR of cDNA. Data are presented as fold induction (over levels at Stage 2) after normalization for cDNA content (quantified by (3-actin levels).

FIG. 45 shows Nurr1 and PitX3 coordinately specify the ‘late’ maturation of ES-derived mDNs. (A) EB-differentiated DY-1 cells were transduced with lentiviral vectors for control vector (DsRed), Nurr1 alone, PitX3 alone, or Nurr1 and PitX3 vectors. Cultures were subsequently fixed and immunostained with antibodies for eYFP and TH; DsRed2 was visualized by confocal microscopy. Scale bar, 50 μm. (B) mDNs from EB differentiated DY-1 ES cultures were immunostained for eYFP, DAT, TuJ1, Serotonin (5-HT), or GABA. Cell numbers were quantified in 10 random fields for each sample by confocal microscopy. Data are presented as mean±s.e.m. and were analyzed by Fischer test (ANOVA). *p<0.05; **p<0.005.

FIG. 46 illustrates Nurr1 and PitX3 play functionally distinct and synergistic roles in mDN maturation. (A-C, E) Quantitative real-time rtPCR analysis of mDN markers as indicated in EB-differentiated DY-1 (A, B, E) or MM13 (C) ES cells transduced with control vector, Nurr1, PitX3, or Nurr1 and PitX3. (A) Expression levels of Nurr1 and PitX3 are dramatically increased by the respective viral vectors, as predicted. (B,C) Nurr1 overexpression is sufficient to induce early markers such as TH, VMAT-2 whereas Nurr1 and PitX3 coordinately induce the late marker DAT. (D) HPLC quantification of dopamine release by EB-differentiated MM13 ES cells after depolarization with a high potassium media (56 mM KCl) for 15 min. PitX3 alone or Nun1 together with PitX3 significantly induced dopamine release. Data are shown as mean±s.e.m. and were analyzed by Fischer test (ANOVA); *p<0.05. (E) Markers for other neuron types such as GABAergic (GAD), and serotonergic neurons (TPH and SERT) are weakly induced by Nurr1 but not by PitX3 or the combination, as determined by quantitative real-time rtPCR. real-time rtPCR levels are presented as a percentage of the value obtained double (Nurr1 and PitX3) transduction after normalization with β3-actin.

FIG. 47 further illustrates PitX3 and Nurr1 coordinately regulate extrinsic factor signaling pathways in EB-differentiated ES cultures. (A) Overexpression of Nurr1 alone is sufficient to induce c-Ret, whereas Nurr1 along with PitX3 additionally induce expression of GFR1a, GDNF, and BDNF in MM13 cultures. (B) PitX3 and Nun1 co-overexpression expands cells that are sensitive to BDNF and GDNF treatment as quantified by rtPCR for the late marker DAT but not the early marker TH. BDNF and GDNF treatment fails to induce expression of Nurr1 and PitX3. Virally transduced MM13 cells were treated with GDNF and BDNF (both 10 ng/ml, from R&D system) during EB stage 4; real-time rtPCR levels are presented as a percentage of the value obtained double (Nurr1 and PitX3) transduction after normalization with P3-actin.

FIG. 48 shows functional analysis of Nurr1 and PitX3 transduced, EB-differentiated mDNs in vivo. EB-differentiated cultures (MM13 Stage 3) transduced with either vector alone (n=4) or Nurr1 and PitX3 (n=5) were transplanted by stereotaxic injection into the striatum of adult mice two weeks after unilateral 6-OHDA lesioning. Engraftment was assessed by immunostaining of striatal sections with TH antibody. (A-:B) The site of engraftment was identified (white circle) at low magnification; TH immunoreactivity was visualized by confocal microscopy in random fields (10 images per mouse, 20×) within 0.6 mm of the injection site. Quantification of imaging was performed by a blinded observer using NIH Image software (Martinat et al., 2004, PLoS in the press). Data represent mean±s.e.m. and were analyzed by Fischer test (ANOVA). *p<0.05. (C) Mice transplanted with the Nurr1 and PitX3-transduced cells displayed significantly fewer contralateral rotations in response to apomorphine (0.4 mg/kg) than vector-transduced cells. Mice were habituated to the environment for 20 min prior to the apomorphine injection (0.1 or 0.4 mg/kg); contralateral rotatory behavior was assessed in hemispheric bowls over a 7 min period by a blinded observer. One animal that had been transplanted with control vector transduced cells displayed severe contralateral barrel rotations in response to 0.4 mg/kg apomorphine) that could not be accurately quantified in this assay and this animal was therefore not included in the statistical analysis. Data were statistically analyzed by the Mann-Whitney test; *, P<0.05.

FIG. 49 demonstrates Lmx1b plays a role the early differentiation of ES-derived mDNs but does not specify the ‘late’ maturation of ES-derived mDNs. (A) Quantitative real-time rtPCR analysis of mDN markers after transduction of MM 13 ES cells with vector alone, Nurr1, Lmx1b or PitX3. Real-time rtPCR levels are presented as a percentage of the value obtained double (Nurri and PitX3) transduction after normalization with (3-actin. (B) Quantification of dopamine release by EB-differentiated MM13 ES cells after depolarization with a high potassium media (56 mM KCl) for 15 min. Data are shown as mean±s.e.m.

FIG. 50 depicts a list of the primers used for the real-time rtPCR analysis.

FIG. 51 shows (A) Quantitative real-time rtPCR analysis of exogenous (virally transduced) Nurr1 and PitX3 expression in differentiated DY-1 ES cells. Primers for virally transduced genes are listed in FIG. 50. (B) Quantitative real-time rtPCR analysis of endogenous and exogenous expression of Nurr1, PitX3, and expression of early mDN markers in EB-differentiated, virally transduced MM13 ES cells. Data are expressed as the percentage of doubly-infected (Nurr1 and PitX3) values after normalization with b-actin. (C) Nurr1 and PitX3 overexpression in undifferentiated DY-1 ES cells does not lead to induction of dopamine neuron markers. Data represent mean±s.e.m and were analyzed by Fischer test (ANOVA). *p<0.05; **p<0.005.

FIG. 52 shows (A) Immuncytochemistry on differentiated MM13 ES cells transduced with Vector (GFP), Nurr1, PitX3 or Nurr1 and PitX3 lentiviral vector. Scale bar, 50 mm. (B) mDNs from EB-differentiated MM13 ES cultures were quantified in 10 random fields for each sample by confocal microscopy. Data are presented as mean±s.e.m. and were analyzed by Fischer test (ANOVA). *p<0.05; **p<0.005.

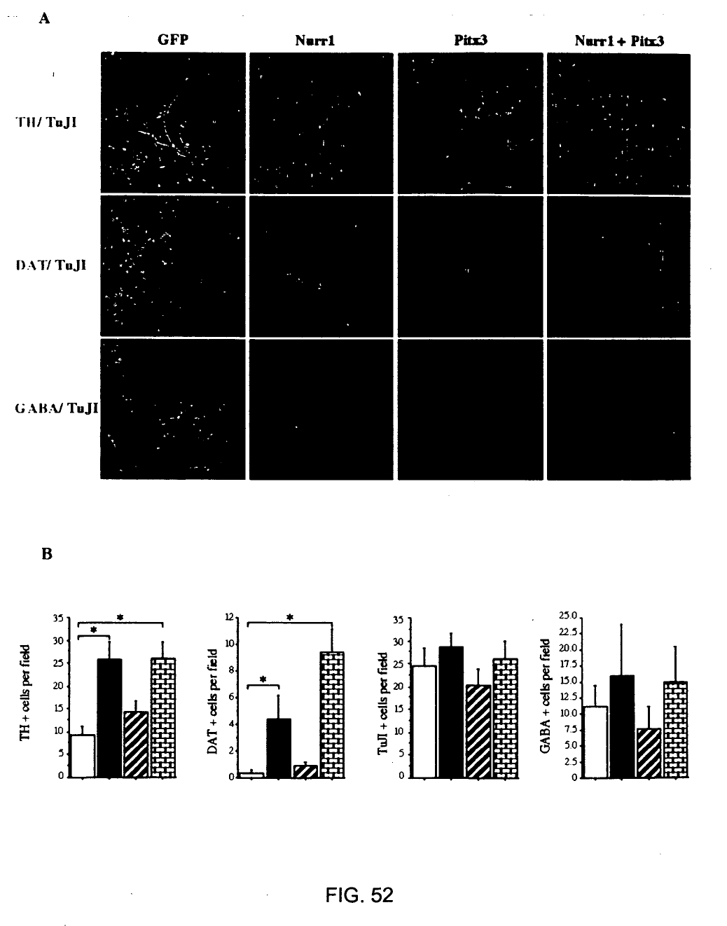

FIG. 53 sets forth immunohistochemical characterization of the infection by lentivirus in the striatum. Four weeks after stereotactic injection of virus in the striatum (volume=3 microliters; rate=0.5 microliters/min; stereotactic coordinate: relative to the bregma for Anteriority: +1 mm and Laterality: +2.2, or to the scull surface for the high: −3 mm), GFP is transduced in a volume corresponding to ⅔ of the structure. (A) represents GFP staining in a striatal section close to the injection site. Cells expressing GFP are positive for NeuN (neuronal specific marker, B) and GAD65 (not shown). The GFP protein is transported anterogradely to the target structures of striatal neurons (globus pallidus, entopedoncular nucleus and substantia nigra pars reticulata, C). GFP in the target structures is found to a lesser extent at earlier time points (1-3 weeks, not shown).

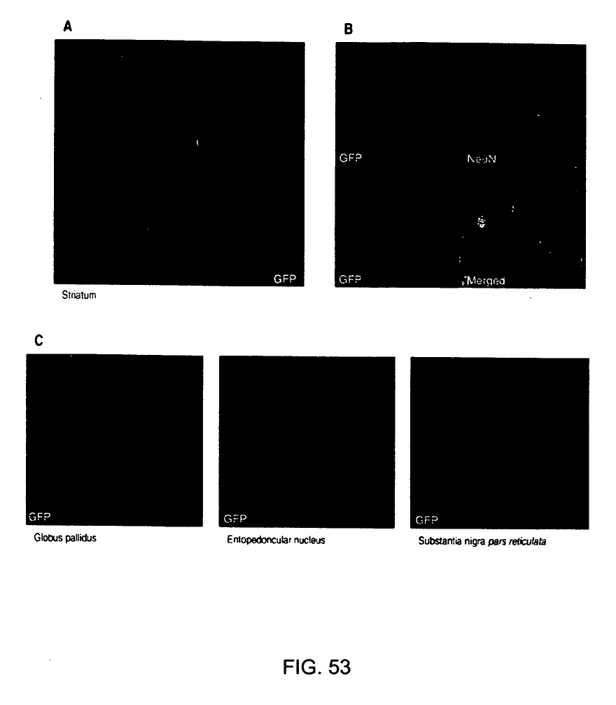

FIG. 54 shows immunohistochemical characterization of the infection by Adeno-associated virus in the substantia nigra pars compacta. Four weeks after stereotactic injection of virus in the substantia nigra pars compacta (volume=2.5 microliters; rate=0.3 microliters/min; stereotactic coordinates: relative to the bregma for Anteriority: −2.9 mm and Laterality: +1.3, or to the scull surface for the high: −4.2 mm), GFP is transduced in about 60 to 80% of tyrosine hydroxylase (TH) positive cells (A). GFP protein is transported anterogradely to the target structures of dopamine neurons (here are shown the striatum, the entopedoncular nucleus and the subthalamic nucleus (B). This expression in the target structures is not found at earlier time points (1-3 weeks, not shown).

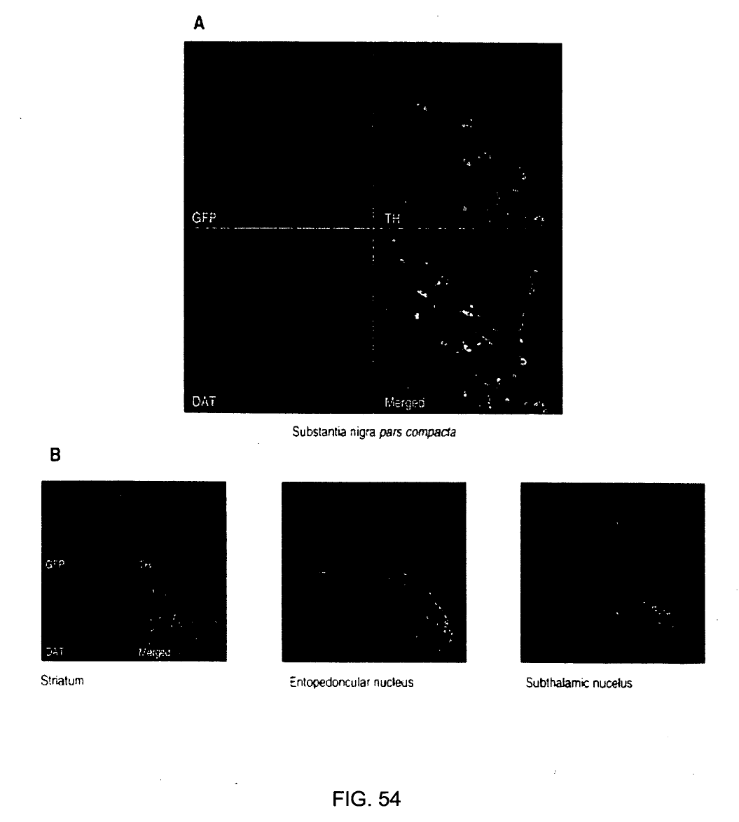

FIG. 55 demonstrates immunohistochemical characterization of the infection by lentivirus in the hippocampus. GFP transduction in the hippocampus four weeks after stereotactic injection of virus (volume=2 microliters; rate=0.2 microliters/min; stereotactic coordinates relative to the bregma for Anteriority: −2.5 mm and Laterality: +2.0 or to the scull surface for the high: −2.0 mm).

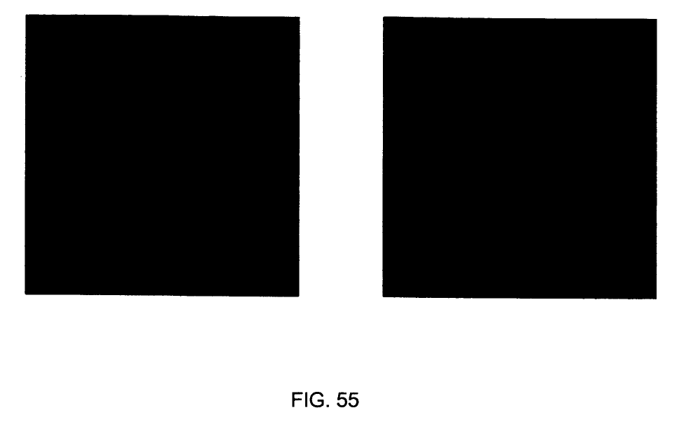

FIG. 56 sets forth immunohistochemical characterization of the infection by adenovirus in the hippocampus. GFP transduction in the hippocampus four weeks after stereotactic injection of virus (volume=2 microliters; rate=0.2 microliters/min; stereotactic coordinates relative to the bregma for Anteriority: −2.5 mm and Laterality: +2.0 or to the scull surface for the high: −2.0 mm).

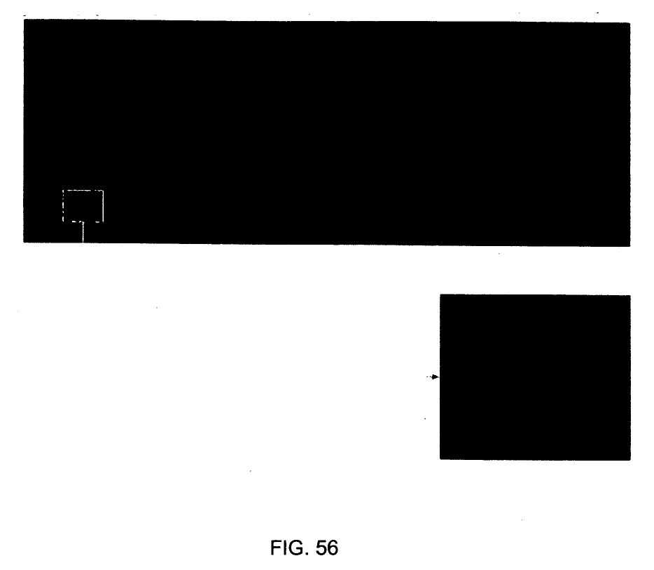

FIG. 57 illustrates immunohistochemical characterization of the infection by Adeno-associated virus in the striatum. GFP transduction in the striatum four weeks after stereotactic injection of virus (volume=3 microliters; rate=0.5 microliters/min; stereotactic coordinates: relative to the bregma for Anteriority: +1 mm and Laterality: +2.2, or to the scull surface for the high: −3 mm).

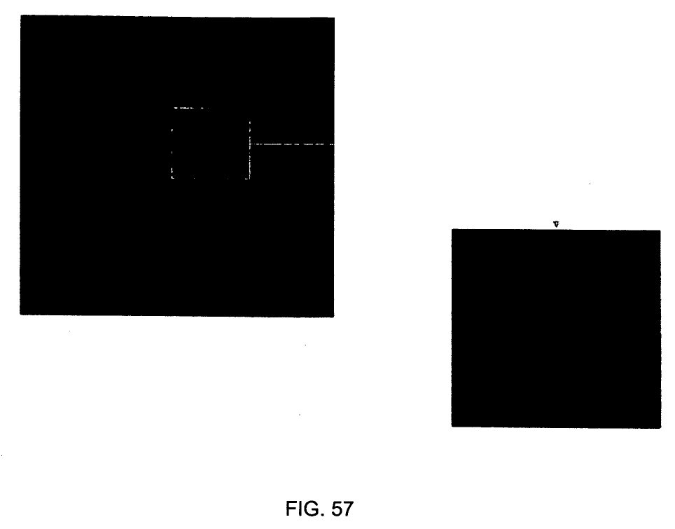

FIG. 58 depicts developmental progression of hES cells using the SDIA method as described (3). (A) Briefly, neural induction was achieved by plating hES cells (1×103 cells/cm2) on a confluent layer of stromal feeder cells in knockout serum replacement medium (KSR), Neural patterning occurred in the presence of 200 ng/ml SHH and 100 ng/ml FGF8b, 20 ng/ml BDNF and 20 ng/ml GDNF (all the factors are from R&D system). At passages 1 and 2, hEScells-derived rosettes were plated on a 15 ug/ml polyornithine/1 ug/ml laminin substrate in the absence of feeder cells. Neural differentiation required the withdrawal of SHH/FGF8b. To increase the dopaminergic differentiation and survival, cells were exposed to 0.2 mM ascorbic acid, 20 ng/ml BDNF, 20 ng/ml GDNF and 1 ng/ml transforming growth factor type b3. (B, C) Lentiviral transduction during the passage 2 led to persistent overexpression of transgene, as indicated by the GFP expression, throughout SDIA differentiation in over 95% of cells.

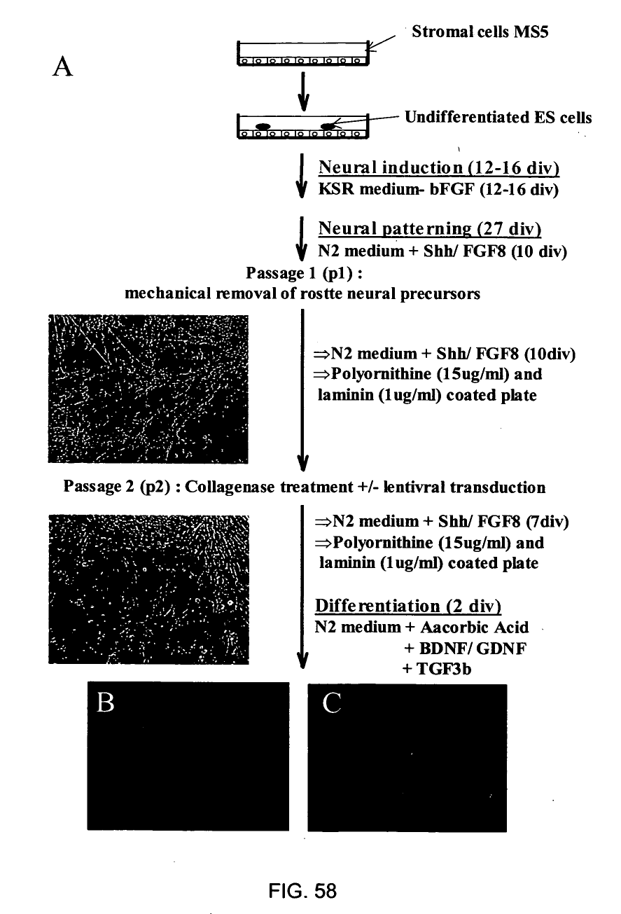

FIG. 59 shows Nurr1 and PitX3 play functionally distinct and synergistic roles in mDN maturation as determined by quantitative real-time rtPCR analysis of mDN markers. SDIA differentiated hES cells (H9 cell line) were transduced with lentiviral vectors for control vector (GFP), Nurr1 alone, PitX3 alone, or Nurr1 and PitX3 vectors at passage 2. (A and B) Expression levels of Nurr1 and PitX3 are dramatically increased by the respective viral vectors, as predicted. (C, D) Nurr1 overexpression is sufficient to induce early markers such as TH whereas Nurr1 and PitX3 coordinately induce the late marker DAT. Quantitative real-time rtPCR. real-time rtPCR levels are presented as a percentage of the value obtained double (Nurr1 and PitX3) transduction after normalization with b-actin.

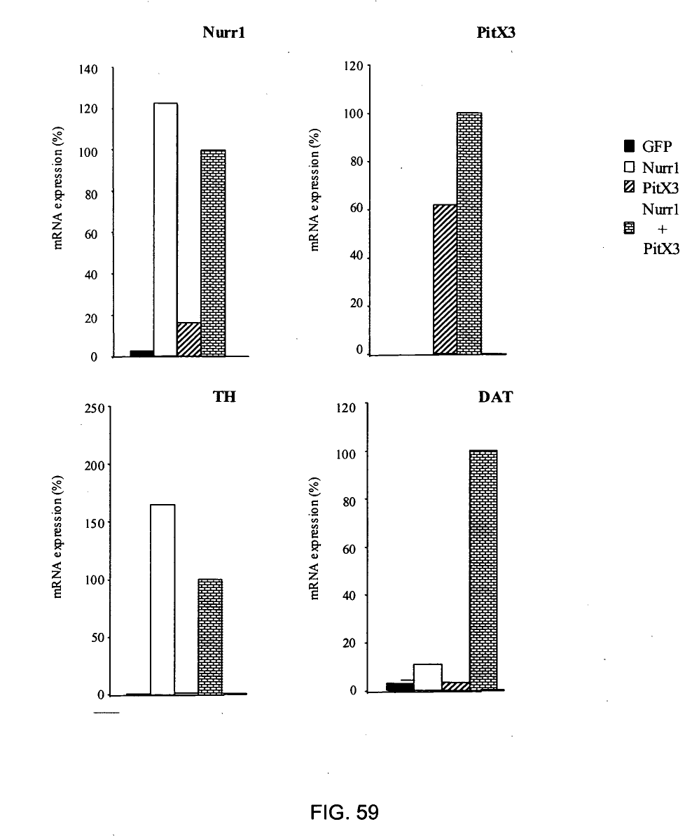

FIG. 60 shows Nurr1 and PitX3 coordinately specify the ‘late’ maturation of ES-derived mDNs. Cultures were subsequently fixed and immunostained with antibodies for the dopaminergic markers TH; DAT and for the pan neuronal marker TuJI as previously described (2). Immunostaining were visualized by confocal microscopy. Scale bar, 50 mm.

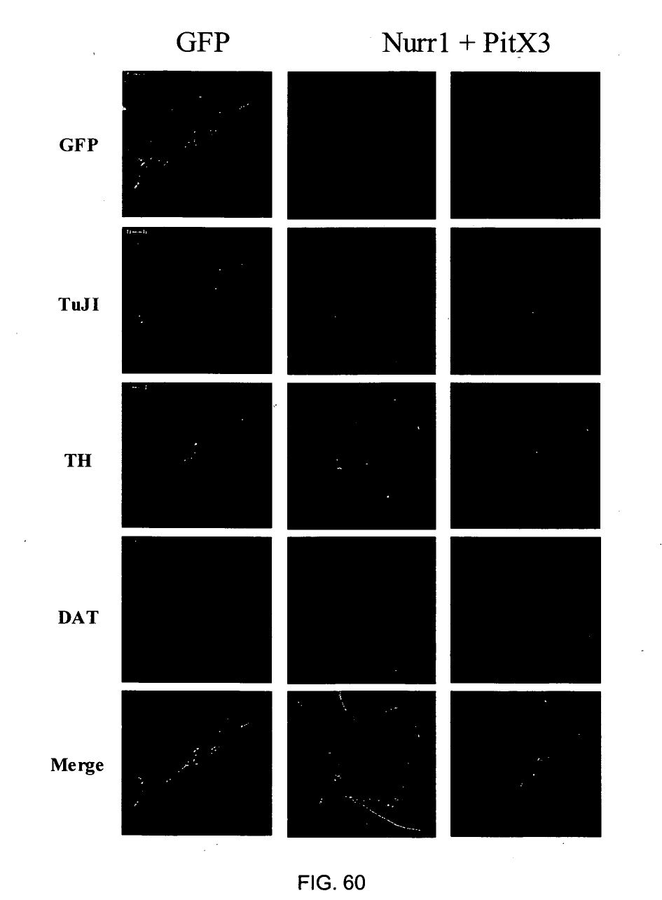

FIG. 61 sets forth a general scheme of hES cells differentiation using the embryoid bodies (EB) protocol (1). (A) hES cells were dissociated using collagenase treatment and allowed to aggregate for 4 days in a non-adherent bacteriological dish at a density of 3×104 cells/cm2. According to the protocol, EB were plated on a 0.1% gelatin-coated dish. Neural precursor cells were selected by incubation in insulin/transferring/selenium/fibronectin medium for 8 days. Then, cells were dissociated and plated onto 6 ug/ml polyornithine/3 ug/ml laminin coated dish at a concentration of 2×105 cells/cm2 and expand in N2 medium containing 1 ug/ml of laminin (Sigma), 10 ng/ml bFGF (Invitrogen), for 6 days. The final differentiation step involved removing bFGF and culturing in N2 medium containing 0.2 mM AA. (B, C) Lentiviral transduction at the end of the stage 3 led to persistent overexpression of transgene, as indicated by the GFP expression, throughout EB protocol in over 95% of cells.

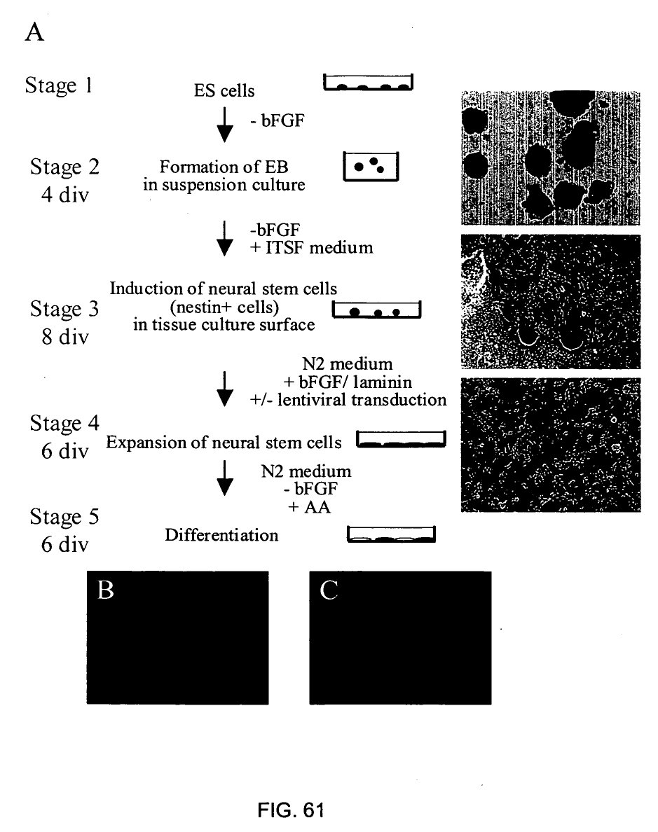

FIG. 62 illustrates quantitative real-time rtPCR analysis of EB-differentiated hES for mDN markers. (A) H9 hES cells were transduced with lentiviral vectors for control vector (GFP), Nurr1 alone, PitX3 alone, or Nurr1 and PitX3 vectors. Expression levels of Nurr1 and PitX3 are dramatically increased by the respective viral vectors, as predicted. Nurr1 overexpression is sufficient to induce early markers such as TH. However, in contrast with the SDIA method, no effect on the expression of TH or DAT is observed after overexpression of Nurr1 and PitX3. (B) Similar results were obtained using an independent hES cell line (BGN2). Quantitative real-time rtPCR. real-time rtPCR levels are presented after normalization with b-actin.

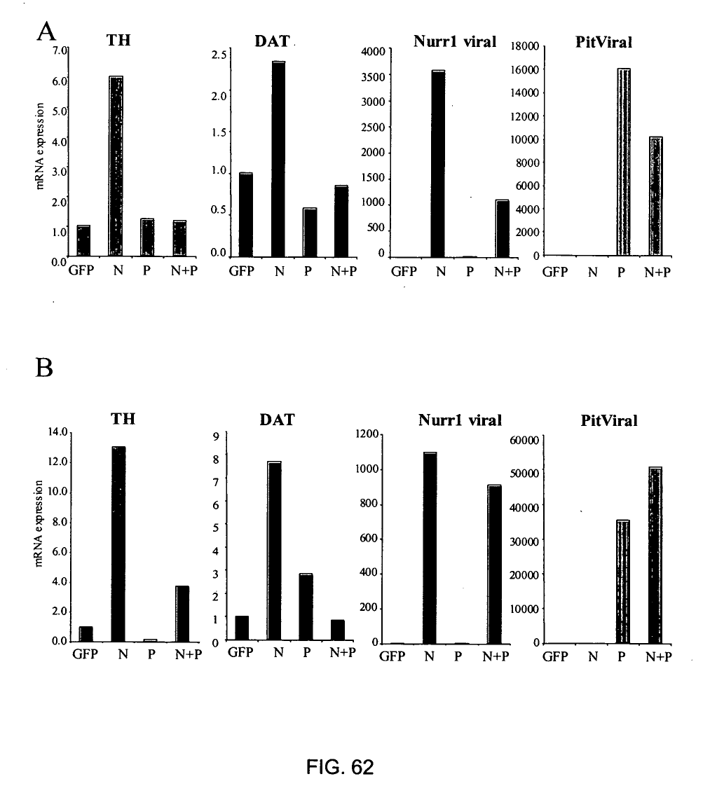

### DETAILED DESCRIPTION OF THE INVENTION

The inventors have developed viral vectors that allow for the over-expression and RNAi mediated knockdown of specific genes in vitro and in vivo, i.e., in midbrain dopamine neurons of a subject. The inventors have disclosed that these viral vectors can be utilized as therapeutic agents in the context of treating or preventing nerurodegeneration, including Parkinson's disease, in a subject, either by overexpression of protective genes or knockdown of toxic genes.

Additionally, the inventors disclose herein that these viral vectors can be used to modify cell-based therapies in order to improve their efficacy. These viral vectors and improved cell therapies can also be used for modifying existing cellular and animal models of neurodegeneration to overcome limitations in these model systems.

Accordingly, the present invention provides compositions and methods for enhancing and improving cell therapies for neurological disorders including Parkinson's disease. Specifically, the present invention provides a cell or cells modified by viral vector compositions that allow for the over-expression and RNAi mediated knockdown of genes in vitro and in vivo. The present invention further provides cell therapy methods for treating or preventing neurodegeneration in a subject, and for protecting neurons from damage in the context of neurodegenerative disorders using the modified cells. Additionally, the present invention provides methods for purification and identification of mature dopamine neurons for cell therapy using fluorescent compounds including JHC1-64.

In one embodiment the invention provides a cultured cell or cells transduced or modified by a composition comprising a nucleic acid encoding a Nurr-1 associated agent and a PitX3-associated agent; a vector; and optionally, a pharmaceutically-acceptable carrier; wherein the Nurr-1-associated agent is selected from the group consisting of a Nurr-1 protein, a Nurr-1 mimetic, a modulator of Nurr-1 expression and a modulator of Nurr-1 activity; and the PitX3-associated agent is selected from the group consisting of a PitX3 protein, a PitX3 mimetic, a modulator of PitX3 expression and a modulator of PitX3 activity. In other specific embodiments of the present invention, the nucleic acid of the composition encodes a parkin-associated agent, a pink-1 associated agent, a DJ-1-associated agent.

The present invention further provides cells transduced or modified by compositions comprising viral vectors that allow for RNAi mediated knockdown of specific genes toxic to neurons. In one embodiment, the invention provides a cell or cells transduced by a composition comprising a nucleic acid having a sequence sufficiently complementary to a portion of an alpha synuclein gene to reduce expression of the gene a vector; and optionally, a pharmaceutically-acceptable carrier wherein the nucleic acid is selected from the group consisting interfering RNA, and shRNA. In another embodiment, the invention provides a cell or cells transduced by a composition comprising a nucleic acid which having a sequence sufficiently complementary to a portion of a park8 gene to reduce expression of the gene a vector; and optionally, a pharmaceutically-acceptable carrier wherein the nucleic acid is selected from the group consisting interfering RNA, and shRNA. In yet another embodiment, the invention provides a cell or cells transduced by a composition comprising a nucleic acid having a sequence sufficiently complementary to a portion of an amyloid precursor protein (APP) gene to reduce expression of the gene a vector; and optionally, a pharmaceutically-acceptable carrier wherein the nucleic acid is selected from the group consisting interfering RNA, and shRNA.

In a particular embodiment of the invention, the vector of the composition also expresses a fluorescent protein, such as green fluorescent protein (GFP). In a preferred embodiment, the vector of the therapeutic composition expresses eGFP.

The cells transduced by the vector compositions of the present invention include but are not necessarily limited to ES cells, neuronal precursor cells, ES-derived dopamine neurons, dopamine neuron precursors and midbrain dopamine neurons. A cell has been transduced within the context of the present invention if it expresses an exogenous nucleic acid molecule.

The present invention additionally provides methods for treating or preventing neurodegeneration in a subject in need of such treatment by administering to the subject the transduced cells of the present invention in an amount effective to treat or prevent the neurodegeneration. In one embodiment, the cells are administered with a pharmaceutically acceptable carrier. The neurodegeneration or neurodegenerative disorder treated or prevented by the method of the present invention includes, but is not necessarily limited to Parkinson's disease (including sporadic Parkinson's disease and autosomal recessive early-onset Parkinson's disease), Alzheimer's disease, stroke, amyotrophic lateral scelerosis, Binswanger's disease, Huntington's chorea, multiple sclerosis, myasthenia gravis, and Pick's disease. In a preferred embodiment of the invention, the neurodegeneration treated or prevented by the compositions and methods of the present invention is Parkinson's disease. In one embodiment of the invention, the cell therapy composition of the invention is used in combination with one or more different cell therapy compositions of the present invention.

In the context of the present invention genes, amino acid or nucleic acid sequences are homologous if they share an arbitrary threshold of similarity determined by alignment of matching bases or amino acids, or have fundamental similarities that indicate a common evolutionary origin. By way of non-limiting example, the human DJ-1 gene has homologs in many different species of mammals and invertebrate species including drosophila and bacteria. Nucleic acid homologs can be produced using techniques known in the art for the production of nucleic acids including, but not limited to, classic or recombinant DNA techniques to effect random or targeted mutagenesis.

“Neuron” as used in the present invention refers to any neuron of the central nervous system (CNS), but is preferably a neuron from the brain. Examples of CNS neurons include, without limitation, cerebellar neurons, or neurons from the cerebellum (e.g., basket cells, Golgi cells, granule cells, Purkinje cells, and stellate cells); cortical neurons, or neurons from the cerebral cortex (e.g., pyramidal cells and stellate cells, including interneurons, midbrain neurons, and neurons of the substantia nigra); hippocampal cells, or cells from the hippocampus (including granule cells); cells of the Pons; and primary neurons (neurons taken directly from the brain, and, in general, placed into a tissue culture dish). Neurons may secrete, or respond to, a variety of neurotransmitters, including, without limitation, acetylcholine, adrenaline, dopamine, endorphins, enkephalins, GABA (gamma aminobutyric acid), glutamate or glutamic acid, noradrenaline, and serotonin. In one embodiment of the present invention, the neuron is a dopamine neuron. Dopamine (3,4-dihydroxyphenylethylamine) is a hormone-like substance with the chemical formula, C₈H₁₁NO₂. It functions in the nervous system as an important neurotransmitter, and is an intermediate in the production of two hormones, epinephrine (adrenaline) and norepinephrine.

The method of the present invention may be used to promote overexpression of genes that protect neurons from toxicity or promote knockdown of toxic genes, in vitro, or in vivo in a subject. As used herein, the “subject” is a mammal, including, without limitation, a cow, dog, human, monkey, mouse, pig, or rat. Preferably, the subject is a human. The cell therapy compositions of the present invention are particularly useful for treating neurodegeneration, particularly parkin-associated neurodegeneration, and neurodegeneration. Accordingly, in one embodiment of the present invention, the subject is a human with neurodegeneration.

As used herein, “neurodegeneration” or “neurodegenerative disorder” refers to a condition of deterioration of nervous tissue, particularly neurons, wherein the nervous tissue changes to a lower or less functionally active form. It is believed that, by over expressing genes that protect neurons from toxicity and/or knockdown of toxic genes, the therapeutic compositions of the present invention will be useful for the treatment of conditions associated with neurodegeneration. It is further believed that use of the therapeutic compositions of the present invention would be an effective therapy, either alone or in combination with other therapeutic agents that are typically used in the treatment of these conditions.

Neurodegeneration may be caused by, or associated with, a variety of factors, including, without limitation, primary neurologic conditions (e.g., neurodegenerative diseases), central nervous system (CNS) and peripheral nervous system (PNS) traumas, and acquired secondary effects of non-neural dysfunction (e.g., neural loss secondary to degenerative, pathologic, or traumatic events, including stroke). Examples of neurodegenerative diseases include, without limitation, Alzheimer's disease, amyotrophic lateral sclerosis (Lou Gehrig's Disease), Binswanger's disease, Huntington's chorea, multiple sclerosis, myasthenia gravis, Parkinson's disease, and Pick's disease. In one embodiment of the present invention, the neurodegeneration is sporadic Parkinson's disease or autosomal recessive early-onset Parkinson's disease. In another embodiment of the present invention, the neurodegeneration is associated with glutamate excitotoxicity.

As used herein, the phrase “effective to treat or prevent the neurodegeneration” means effective to ameliorate or minimize the clinical impairment or symptoms resulting from the neurodegeneration. For example, where the neurodegeneration is Parkinson's disease, the clinical impairment or symptoms of the neurodegeneration may be ameliorated or minimized by diminishing any pain or discomfort suffered by the subject; by extending the survival of the subject beyond that which would otherwise be expected in the absence of such treatment; by inhibiting or preventing the development or spread of the neurodegeneration, including loss, in the substantia nigra, of nerve cells containing dopamine; and/or by limiting, suspending, terminating, or otherwise controlling tremors, speech impediments, movement difficulties, dementia, and other symptoms associated with Parkinson's disease. The amount of the therapeutic composition that is effective to treat neurodegeneration in a subject will vary depending on the particular factors of each case, including the type of neurodegeneration, the stage of neurodegeneration, the subject's weight, the severity of the subject's condition, and the method of administration. These amounts can be readily determined by the skilled artisan.

In a specific embodiment of the invention, the therapeutic cell composition is administered directly into the brain of a subject. The therapeutic cell compositions of the present invention can be directly administered to any structure in the brain. In one embodiment, the compositions are administered to brain structures selected from the group consisting of substantia nigra, hippocampus, striatum, and cortex. In a preferred embodiment of the invention, the cell composition is administered using a stereotactic device.

Also provided are methods for use of the cell compositions of the invention to improve an animal model of Parkinson's disease or other neurodegenerative disorder. In one embodiment, a cell composition is provided, comprising a nucleic acid comprising a sequence sufficiently complementary to a portion of a gene selected from the group consisting of PAD1, Psmc4, Apg7L and NPC, to reduce expression of the gene a vector; and optionally, a pharmaceutically-acceptable carrier wherein the nucleic acid is selected from the group consisting interfering RNA, and shRNA. In another embodiment, the vector expresses a fluorescent protein, including but not limited to green fluorescent protein, and the vector is selected from the group consisting of an adeno-associated viral vector or a lentiviral vector. In still another embodiment, the animal model of neurodegeneration or neurodegenerative disorder includes but is not necessarily limited to Parkinson's disease, autosomal recessive early-onset Parkinson's disease, Alzheimer's disease, stroke, amyotrophic lateral scelerosis, Binswanger's disease, Huntington's chorea, multiple sclerosis, myasthenia gravis, and Pick's disease.

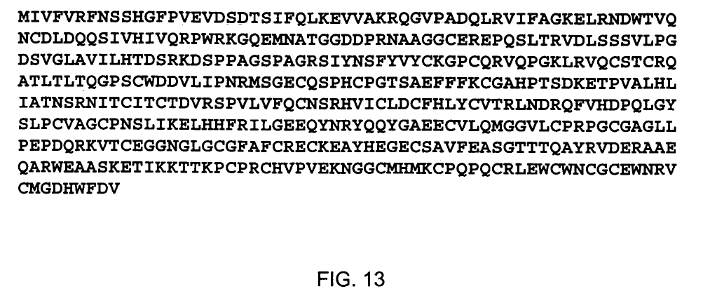

Unless otherwise indicated, “parkin” “pink-1 and “DJ-1” refer to and include both peptides and analogues. By way of a non-limiting example, “parkin peptide” includes at least the carboxyl terminus domain of parkin (including conservative substitutions thereof), from residues 76-465, up to and including a “parkin protein” having the amino acid sequence set forth in FIG. 13 (including conservative substitutions thereof). Unless otherwise indicated, “protein” shall include a protein, protein domain, polypeptide, or peptide. A “parkin analogue”, for example is a functional variant of the parkin peptide, having parkin biological activity, that has 60% or greater (preferably, 70% or greater) amino-acid-sequence homology with the parkin peptide. As further used herein, the term “peptide biological activity” refers to the activity of a protein or peptide that demonstrates an ability to associate physically with, or bind with, hSel-10 (i.e., binding of approximately two fold, or, more preferably, approximately five fold, above the background binding of a negative control), under the conditions of the assays described herein, although affinity may be different from that of parkin.

It will be obvious to the skilled practitioner that the numbering of amino acid residues in parkin, pink-1 or DJ-1 or in the parkin, pink-1 or DJ-1 analogues or mimetics covered by the present invention, may be different than that set forth herein, or may contain certain conservative amino acid substitutions that produce the same associating activity as described herein. Corresponding amino acids and conservative substitutions in other isoforms or analogues are easily identified by visually inspecting the relevant amino acid sequences, or by using commercially available homology software programs.

The parkin-associated complex of the present invention has ubiquitin ligase activity, and can promote ubiquitination of cellular substrates, including cyclin E. Thus, in one embodiment, the parkin-associated complex further comprises cyclin E.

In the method of the present invention, the therapeutic cell composition may be administered to a human or animal subject by known procedures, including, without limitation, oral administration, parenteral administration (e.g., epifascial, intracapsular, intracutaneous, intradermal, intramuscular, intraorbital, intraperitoneal, intraspinal, intracranial, intrasternal, intravascular, intravenous, parenchymatous, or subcutaneous administration), transdermal administration, and administration by osmotic pump. One preferred method of administration is parenteral administration, by intravenous or subcutaneous injection.

For oral administration, the therapeutic cell composition may be presented as capsules, tablets, powders, granules, or as a suspension. The formulation may have conventional additives, such as lactose, mannitol, cornstarch, or potato starch. The formulation also may be presented with binders, such as crystalline cellulose, cellulose derivatives, acacia, cornstarch, or gelatins. Additionally, the formulation may be presented with disintegrators, such as cornstarch, potato starch, or sodium carboxymethylcellulose. The formulation also may be presented with dibasic calcium phosphate anhydrous or sodium starch glycolate. Finally, the formulation may be presented with lubricants, such as talc or magnesium stearate.

For parenteral administration, the cell therapy composition may be combined with a sterile aqueous solution, which is preferably isotonic with the blood of the subject. Such a formulation may be prepared by dissolving a solid active ingredient in water containing physiologically-compatible substances, such as sodium chloride, glycine, and the like, and having a buffered pH compatible with physiological conditions, so as to produce an aqueous solution, then rendering said solution sterile. The formulation may be presented in unit or multi-dose containers, such as sealed ampules or vials. The formulation also may be delivered by any mode of injection, including any of those described above.

For transdermal administration, the cell therapy composition may be combined with skin penetration enhancers, such as propylene glycol, polyethylene glycol, isopropanol, ethanol, oleic acid, N-methylpyrrolidone, and the like, which increase the permeability of the skin to the modulator, protein, or nucleic acid, and permit the modulator, protein or nucleic acid to penetrate through the skin and into the bloodstream. The composition of enhancer and modulator, protein, or nucleic acid also may be further combined with a polymeric substance, such as ethylcellulose, hydroxypropyl cellulose, ethylene/vinylacetate, polyvinyl pyrrolidone, and the like, to provide the composition in gel form, which may be dissolved in solvent, such as methylene chloride, evaporated to the desired viscosity, and then applied to backing material to provide a patch. The cell therapy composition may be administered transdermally, at or near the site on the subject where the neuron of interest is located. Alternatively, the modulator, protein, or nucleic acid may be administered transdermally at a site other than the affected area, in order to achieve systemic administration.

The cell therapy composition of the present invention also may be released or delivered from an osmotic mini-pump or other time-release device. The release rate from an elementary osmotic mini-pump may be modulated with a microporous, fast-response gel disposed in the release orifice. An osmotic mini-pump would be useful for controlling release, or targeting delivery, of the modulator, protein, or nucleic acid.

The cell therapy composition of the present invention may be administered or introduced to a subject by known techniques used for the introduction of proteins, nucleic acids, and other drugs, including, for example, injection and transfusion. Where the neurodegeneration is localized to a particular portion of the body of the subject, it may be desirable to introduce the cell therapy composition directly to that area by injection or by some other means (e.g., by introducing the cell therapy composition into the blood or another body fluid). In an embodiment, the therapeutic compositions of the present invention are injected into the brain of a subject using stereotactic techniques well-known to the skilled artisan. The amount of the cell therapy composition to be used is an amount effective to treat neurodegeneration in the subject, as defined above, and may be readily determined by the skilled artisan.

The nucleic acids of the present invention, including those encoding the parkin-associated agent, the pink-associated agent, the DJ-1 associated agent, all may be introduced to the subject using conventional procedures known in the art, including, without limitation, electroporation, DEAE Dextran transfection, calcium phosphate transfection, lipofection, monocationic liposome fusion, polycationic liposome fusion, protoplast fusion, creation of an in vivo electrical field, DNA-coated microprojectile bombardment, injection with recombinant replication-defective viruses, homologous recombination, in vivo gene therapy, ex vivo gene therapy, viral vectors, and naked DNA transfer, or any combination thereof. Recombinant viral vectors suitable for gene therapy include, but are not limited to, vectors derived from the genomes of viruses such as retrovirus, HSV, adenovirus, adeno-associated virus, Semiliki Forest virus, lentivirus, cytomegalovirus, and vaccinia virus. In a preferred embodiment of the present invention, the cell therapy compositions comprise an adenovirus vector or a lentivirus vector.

It is within the confines of the present invention that a nucleic acids of the present invention may be introduced into suitable neurons in vitro, using conventional procedures, to achieve expression of the therapeutic protein in the neurons. Neurons expressing the nucleic acids, then may be introduced into a subject to treat neurodegeneration in vivo. In such an ex vivo gene therapy approach, the neurons are preferably removed from the subject, subjected to DNA techniques to incorporate nucleic acid encoding the particular therapeutic protein, and then reintroduced into the subject.

It is also within the confines of the present invention that a formulation containing a transduced cell of the invention, may be associated with a pharmaceutically-acceptable carrier, thereby comprising a pharmaceutical composition. Accordingly, the present invention further provides a pharmaceutical composition, comprising the cell therapy composition of the present invention, and a pharmaceutically acceptable carrier. The pharmaceutically acceptable carrier must be “acceptable” in the sense of being compatible with the other ingredients of the composition, and not deleterious to the recipient thereof. The pharmaceutically acceptable carrier employed herein is selected from various organic or inorganic materials that are used as materials for pharmaceutical formulations, and which may be incorporated as analgesic agents, buffers, binders, disintegrants, diluents, emulsifiers, excipients, extenders, glidants, solubilizers, stabilizers, suspending agents, tonicity agents, vehicles, and viscosity-increasing agents. If necessary, pharmaceutical additives, such as antioxidants, aromatics, colorants, flavor-improving agents, preservatives, and sweeteners, may also be added. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc, and water, among others.

The pharmaceutical composition of the present invention may be prepared by methods well-known in the pharmaceutical arts. For example, the composition may be brought into association with a carrier or diluent, as a suspension or solution. Optionally, one or more accessory ingredients (e.g., buffers, flavoring agents, surface active agents, and the like) also may be added. The choice of carrier will depend upon the route of administration of the vaccine. Formulations of the composition may be conveniently presented in unit dosage, or in such dosage forms as aerosols, capsules, elixirs, emulsions, eye drops, injections, liquid drugs, pills, powders, granules, suppositories, suspensions, syrup, tablets, or troches, which can be administered orally, topically, or by injection, including, but not limited to, intravenous, intraperitoneal, subcutaneous, and intramuscular injection.

In one embodiment of the present invention, the pharmaceutical composition is a therapeutic composition comprising a nucleic acid encoding a parkin-associated agent (e.g., a parkin protein, a parkin mimetic, a modulator of parkin expression, and a modulator of parkin activity), a lentiviral vector, and, optionally, a pharmaceutically-acceptable carrier. By way of example, a parkin lentiviral vector may be assembled by cloning the human parkin cDNA into the BamH1 and XhoI restriction enzyme sites of plasmid pTRIP GFP, and replacing the GFP gene (Zennou et al., The HIV-1 DNA flap stimulates HIV vector-mediated cell transduction in the brain. Nat. Biotechnol., 19:446-50, 2001). A parkin virus may be produced by co-transfection of 293T cells with p8.91 and pHCMV-G, and viral transduction of neuronal cultures may be performed as described (Zennou et al., The HIV-1 DNA flap stimulates HIV vector-mediated cell transduction in the brain. Nat. Biotechnol., 19:446-50, 2001).

The formulations of the present invention may be prepared by methods well-known in the pharmaceutical arts. For example, the vector of the present invention may be brought into association with a carrier or diluent, as a suspension or solution. Optionally, one or more accessory ingredients (e.g., buffers, flavoring agents, surface active agents, and the like) also may be added. The choice of carrier will depend upon the route of administration. The pharmaceutical composition would be useful for administering the therapeutic composition to a subject to treat neurodegeneration. The therapeutic composition is provided in an amount that is effective to treat neurodegeneration in a subject to whom the pharmaceutical composition is administered. That amount may be readily determined by the skilled artisan, as described above.

In accordance with the method of the present invention, the therapeutic cell composition may be administered to a subject who has neurodegeneration, either alone or in combination with one or more drugs used to treat or prevent neurodegeneration, including Parkinson's disease. Examples of drugs used to treat Parkinson's disease include, without limitation, deprenyl, selenium, and vitamin E.

The therapeutic cell composition is provided in an amount that is effective to treat the neurodegeneration in a subject to whom the composition is administered. This amount may be readily determined by the skilled artisan. In one embodiment of the present invention, the cell composition comprises a nucleic acid encoding a parkin-associated agent (e.g., a parkin protein, a parkin mimetic, a modulator of parkin expression, and a modulator of parkin activity), a lentiviral vector, and, optionally, a pharmaceutically-acceptable carrier. In a preferred embodiment of the present invention, the neurodegeneration is sporadic Parkinson's disease or autosomal recessive early-onset Parkinson's disease.

The cell therapy compositions of the present invention may also be useful for studying treatment options in animal models of neurodegeneration, including Parkinson's disease. In particular, because lentivirus vectors are potentially useful, in vivo, for gene therapy, the present invention may provide an animal model demonstrating the efficacy of using parkin-encoding lentivirus in Parkinson's disease. Accordingly, the present invention also provides for use of the pharmaceutical composition of the present in an animal model of neurodegeneration (e.g., Parkinson's disease). In one embodiment of the present invention, the cell composition comprises a nucleic acid encoding a parkin-associated agent (e.g., a parkin protein, a parkin mimetic, a modulator of parkin expression, and a modulator of parkin activity), a lentiviral vector, and, optionally, a pharmaceutically-acceptable carrier. In a preferred embodiment of the present invention, the neurodegeneration is sporadic Parkinson's disease or autosomal recessive early-onset Parkinson's disease.

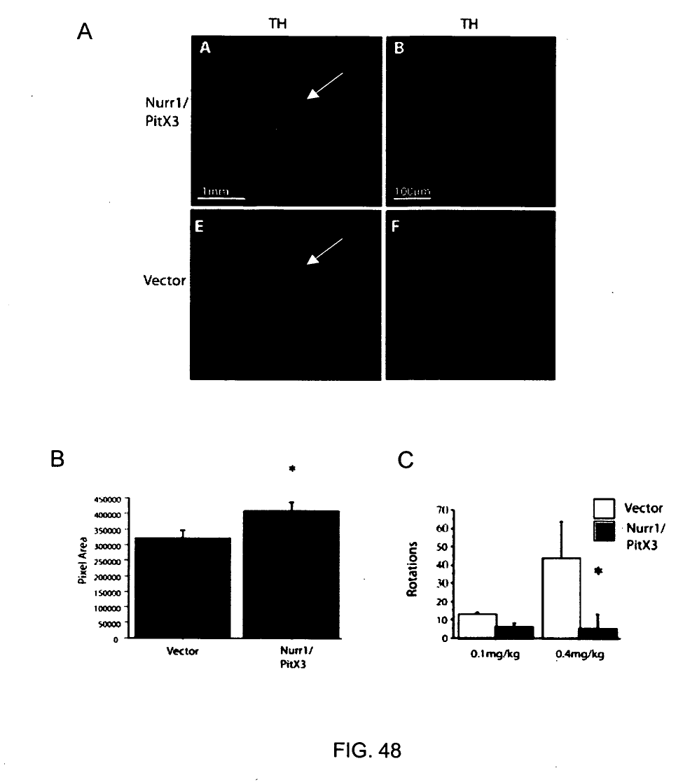

The inventors further disclose herein that fluorescent dye JHC 1-64 (C₅₀H₆₁Cl₂N₅O₉S₂) (FIG. 48), when used in conjunction with Fluorescent Activated Cell Sorting (FACS), is valuable for identifying mature dopamine neurons and purifying mature dopamine neurons from immature ES cells or other populations in cell therapies. This discovery is critical for successful cell therapies because immature or otherwise contaminating cells can lead to tumors. Purification of mature cells leads to more efficacious treatment. Accordingly, the present invention also provides methods for preparing a preparation of mature dopamine neurons comprising contacting a cell sample with at least one fluorescent compound that targets the dopamine transporter protein; identifying cells containing the dopamine transporter protein, and removing cell and cellular aggregates that do not contain the dopamine transporter protein by FACS analysis; wherein the resulting preparation consists essentially of cells containing the dopamine transporter protein. A combination of more than one fluorescent compound can be used in the method of the invention. In a preferred embodiment, at least one of the fluorescent compounds is JHC 1-64. By way of non-limiting example, the cell sample may contain embryonic stem (ES) cells, neuronal precursor cells, ES derived dopamine neurons, dopamine neuron precursor cells, and dopamine neurons.

The present invention is described in the following Examples, which are set forth to aid in the understanding of the invention, and should not be construed to limit in any way the scope of the invention as defined in the claims which follow thereafter.

### EXAMPLES

### Example 1

Expression Vectors, Cell Cultures and Antibodies

cDNAs for parkin, UbcH7, α-synuclein, and UbcH8 were PCR-amplified from a human liver cDNA library (Clontech), and cloned into the eukaryotic expression vectors, pCMS-EGFP (Clontech) or pcDNA3.1. Flag-parkin, T240R parkin, Flag-T240R parkin, and ΔUHD parkin were generated by PCR-mediated mutagenesis. A cDNA clone encoding PP2A/Bα was obtained from Research Genetics. HSel-10 constructs have been described (Wu et al., SEL-10 is an inhibitor of Notch signaling that targets Notch for ubiquitin-mediated protein degradation. Mol. Cell Biol., 21:7403-15, 2001). The integrity of all constructs was confirmed by automated sequencing. Recombinant baculoviruses expressing GST-tagged parkin were generated using the Baculogold system (Pharmingen), as per the manufacturer's instructions.

HeLa cells were maintained in Dulbecco's Modified Eagle Medium (Life Technologies), supplemented with 10% fetal bovine serum (Life Technologies), and heat-inactivated for 30 min at 50° C. Cells were transfected using Lipofectamine Plus (Life Technologies), incubated for 24-36 h, and treated as appropriate with 2.5 μM lactacystin (Sigma) for 16 h. Baculovirus expression and protein purifications were performed as described (Carrano et al., SKP2 is required for ubiquitin-mediated degradation of the CDK inhibitor p27. Nat. Cell Biol., 1:193-99, 1999).

A parkin lentiviral vector was assembled by cloning the human parkin cDNA into the BamH1 and XhoI restriction enzyme sites of plasmid pTRIP GFP, and replacing the GFP gene (Zennou et al., The HIV-1 DNA flap stimulates HIV vector-mediated cell transduction in the brain. Nat. Biotechnol., 19:446-50, 2001). Parkin and control GFP viruses were produced by co-transfection of 293T cells with p8.91 and pHCMV-G; viral transduction of neuronal cultures was performed as described (Zennou et al., The HIV-1 DNA flap stimulates HIV vector-mediated cell transduction in the brain. Nat. Biotechnol., 19:446-50, 2001).

Parkin and cleaved-PARP polyclonal antibodies were obtained from Cell Signaling; α-Synuclein, UbH7, and Skp1 monoclonal antibodies were obtained from Transduction Labs; monoclonal rat antibody against DAT, and polyclonal rabbit antibodies against PP2A-Bα and GAD-65, were obtained from Chemicon; HA polyclonal antibody was obtained from Clontech; HRP-coupled Flag monoclonal antibody was obtained from Sigma; Myc polyclonal, cyclin D1 polyclonal, cyclin A1 polyclonal, and cyclin E monoclonal and polyclonal antibodies were obtained from Santa Cruz; Cul-1 and Rbx1 polyclonal antibodies were obtained from Zymed; and hSel-10 (69 kDa form) polyclonal antibody was obtained from Gentaur Molecular Products. Mouse monoclonal antibody, 2E10, against recombinant human parkin was generated using standard techniques (Ericson et al., Two critical periods of Sonic Hedgehog signaling required for the specification of motor neuron identity. Cell, 87:661-73, 1996). HRP-coupled goat-anti-mouse and goat-anti-rabbit secondary antibodies were obtained from Jackson Immunoresearch. Fluorescently-labeled secondary antibodies were obtained from Molecular Probes.

Immunoprecipitation, Western Blot and mRNA Analysis

Transiently transfected HeLa cells were suspended in lysis buffer (50 mM Tris (pH 7.6), 150 mM NaCl, 0.2% Triton X-100, and complete protease inhibitors (Sigma)), incubated for 60 min at 4° C., and cleared by centrifugation at 20,000×g for 15 min at 4° C. Samples used for in vivo ubiquitination assays were suspended in lysis buffer supplemented with 2.5 mM N-ethyl maleimide (NEM). Lysates were subsequently quenched with 2.5 mM DTT for 20 min at 4° C. Immunoprecipitations and Western blotting were performed using standard techniques (Wu et al., SEL-10 is an inhibitor of Notch signaling that targets Notch for ubiquitin-mediated protein degradation. Mol. Cell Biol., 21:7403-15, 2001). Human brain tissue was obtained from the Columbia University Alzheimer's Disease Research Center Brain Bank. Quantitative real-time PCR was performed as described (Troy et al., Death in the balance: alternative participation of the caspase-2 and -9 pathways in neuronal death induced by nerve growth factor deprivation. J. Neurosci., 21:5007-16, 2001) using primers specific for cyclin E and β-actin.

Banked Brain Tissue Analysis

ARPD mutant brain tissue was identified by genotyping of banked, early-onset PD brains for parkin mutations. One sample showed a 40-bp deletion in exon 3 (Δ438-477) in one allele of parkin, and a complete deletion of exon 8 in the other. Pathological analysis demonstrated depigmented substantia nigra without Lewy bodies (data not shown). Tissue was processed as below.

Pull-Down and Ubiquitination Assays

Brain tissue (2 g per pulldown, maintained at 4° C.) was homogenized in 3× volume buffer (150 mM NaCl, 50 mM Tris (pH 7.6)), and centrifuged at 1,000×g for 15 min. 0.2% Triton X-100 was added to supernatants, and samples were centrifuged at 20,000×g for 20 min. Thereafter, samples were incubated with either parkin monoclonal-antibody-conjugated agarose beads (Pierce), or anti-Flag antibody-conjugated agarose beads (Sigma), along with recombinant Flag-hSel-10, for 2 h. Beads were washed five times with lysis buffer, and protein was eluted with LDS loading buffer (Life Technologies). In vitro ubiquitination assays were performed as described (Koepp et al., Phosphorylation-dependent ubiquitination of cyclin E by the SCFFbw7 ubiquitin ligase. Science, 294:173-77, 2001). Purified yeast E1, human UbcH7, ubiquitin, and ubiquitin aldehyde were obtained from Boston Biochem.

Neuronal Assays

Cerebellar granule neurons from P6 mice were purified and transfected essentially as described (Scheiffele et al., Neuroligin expressed in nonneuronal cells triggers presynaptic development in contacting axons. Cell, 101:657-69, 2000). Dissociated cortical neurons from E16.5 mice were prepared and cultured as described (Scheiffele et al., Neuroligin expressed in nonneuronal cells triggers presynaptic development in contacting axons. Cell, 101:657-69, 2000). Primary midbrain cultures were prepared from E13.5 mouse embryos, as described (Hynes et al., Induction of midbrain dopaminergic neurons by Sonic hedgehog. Neuron, 15:35-44, 1995). Cells were treated for 24 h with or without kainate (250 or 500 μM as indicated; Sigma) or MPP⁺ (1 or 10 μM as indicated; Sigma), stained for 20 min with 0.5 μg/ml Hoechst 33342 (Sigma), and visualized by fluorescence microscopy. For granule cell assays, at least 100 cells in two 20× fields were scored in duplicate for GFP and/or Hoechst signal. For immunohistochemistry, cell cultures were washed twice with PBS, and fixed for 20 min with 4% (w/v) paraformaldehyde. Cells were blocked for 1 h, at room temperature, with 3% (v/v) goat serum in PBS, and then incubated overnight, at 4° C., with specific antibodies, as indicated. Cells were washed, incubated for 1 h at room temperature with appropriately-labeled secondary antibodies, and visualized by fluorescence microscopy. For midbrain culture experiments, DAT-specific immunoreactivity (pixels), and cytoplasmic parkin and cyclin E immunoreactivity (mean pixel density), were quantified in triplicate, across nine fields of view, at 20× using Image software (Scion).

SiRNAs were synthesized by Dharmacon Research, Inc., and duplexes were formed as per the manufacturer's instructions (parkin siRNA sequence: 5′ UUCCAAACCGG AUGAGUGGdTdT 3′; DAT siRNA sequence: 5′ GAGCGGGAGACCUGGAGCAdTdT 3′; SERT siRNA sequence: 5′CUCCUGGAACACUGGCAACdTdT 3′). Cortical cultures were transfected using Lipofectamine 2000 reagent (Life Technologies); primary midbrain cultures were transfected using Transmessenger (Qiagen), as described (Krichevsky and Kosik, RNAi functions in cultured mammalian neurons. Proc. Natl. Acad. Sci. USA, 99:11926-929, 2002).

Discussed below are results obtained by the inventors in connection with the experiments of Examples 1-5:

Parkin Interacts with HSel-10, an F-Box/WD-Repeat Domain Protein

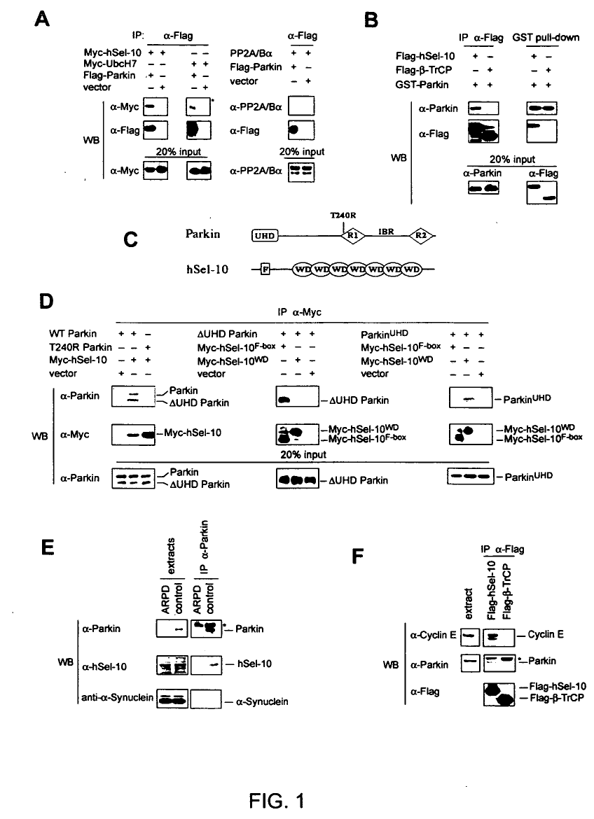

Epitope-tagged parkin and candidate interacting proteins were co-expressed in insect or HeLa cells; complexes were isolated by pull-down assays, and subsequently analyzed by Western blotting. Parkin was found to associate with hSel-10, an F-box/WD protein, in both the HeLa cell (FIG. 1A) and insect cell (FIG. 1B) systems. In contrast, parkin failed to associate with β-TrCP, a second F-box/WD protein (FIG. 1B). Parkin also failed to associate with several other WD-repeat-containing proteins (protein phosphatase 2A/Bα, the β subunit of heterotrimeric protein, and the Cockayne syndrome subunit A gene product) or F-box proteins (FIG. 1A and data not shown). Of note, hSel-10 and parkin are both predominantly cytoplasmic proteins enriched in adult brain (Koepp et al., Phosphorylation-dependent ubiquitination of cyclin E by the SCFFbw7 ubiquitin ligase. Science, 294:173-77, 2001). Parkin has also been reported to co-localize with Golgi apparatus and synaptic markers (Fallon et al., Parkin and CASK/LIN-2 associate via a PDZ-mediated interaction and are co-localized in lipid rafts and postsynaptic densities in brain. J. Biol. Chem., 25:25, 2001; Kubo et al., Parkin is associated with cellular vesicles. J. Neurochem., 78:42-54, 2001). Co-transfection of Flag-parkin and Myc-tagged UbcH7, followed by Flag immunoprecipitation, confirmed the previously-described association of parkin and the E2 UbcH7 (FIG. 1A) (Shimura et al., Familial Parkinson disease gene product, parkin, is a ubiquitin-protein ligase. Nat. Genet., 25:302-05, 2000), whereas no such interaction was observed with other E2s, including UbcH8 and Ubc6 (data not shown).

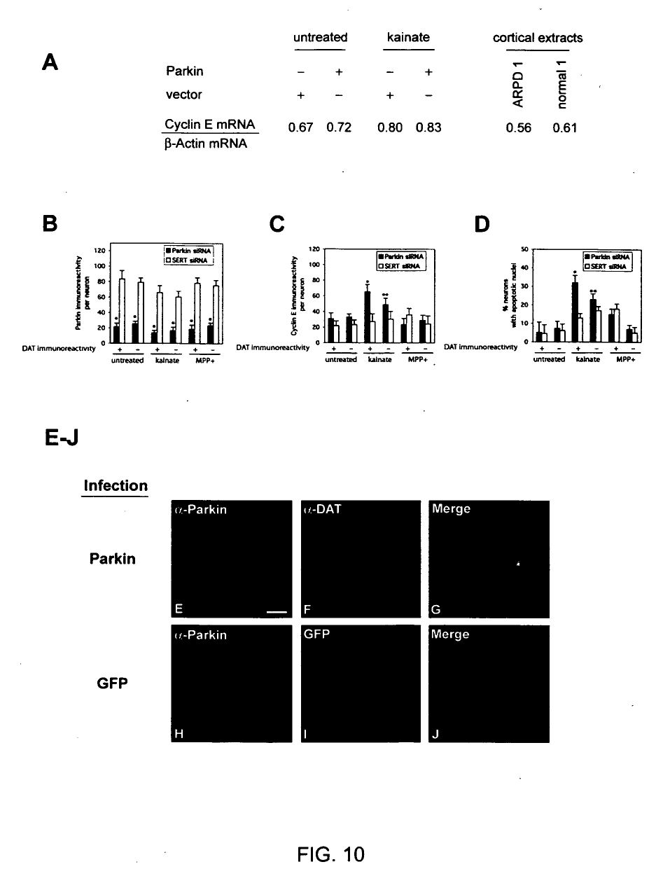

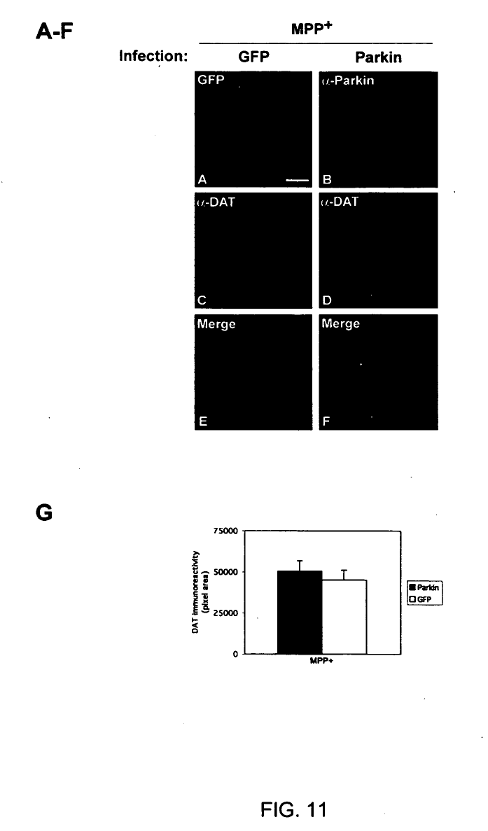

Deletion analysis of parkin and hSel-10 in transfected HeLa cells revealed that the carboxyl terminus of parkin, which includes the two RING finger domains, interacts specifically with the F-box of hSel-10 (FIGS. 1C and 1D). Furthermore, a missense mutation in parkin within the first RING finger (T240R; FIG. 1C), which leads to a familial ARPD syndrome (Shimura et al., Familial Parkinson disease gene product, parkin, is a ubiquitin-protein ligase. Nat. Genet., 25:302-05, 2000), abrogated the interaction with hSel-10 (FIG. 1D), consistent with the notion that this association is important for parkin function. A second interaction was apparent between either full-length or the amino-terminal ubiquitin homology domain (UHD) of parkin and the WD-repeat domain of hSel-10, but this interaction was not required for parkin-hSel-10 association (FIG. 1D and FIGS. 9-11).

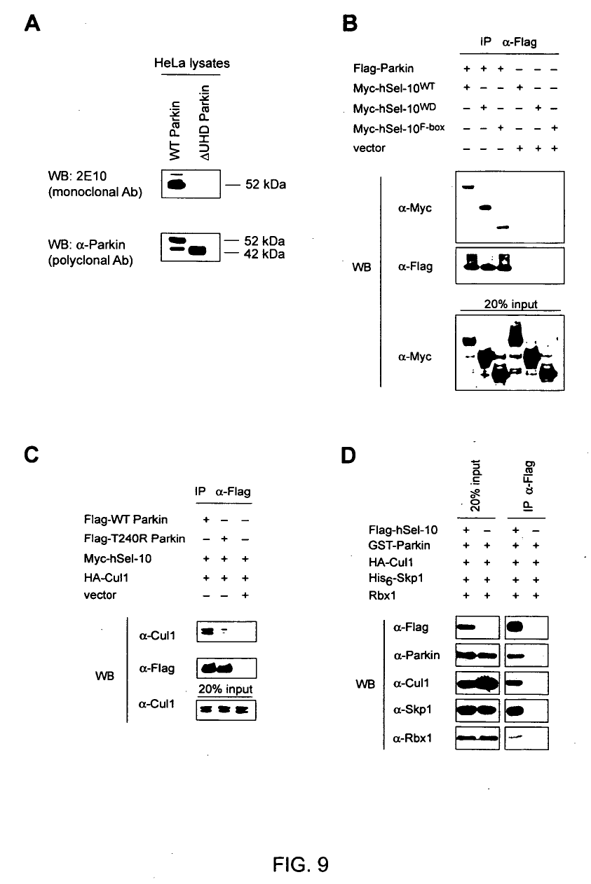

The inventors sought to confirm the interaction between parkin and hSel-10 in mammalian brain extracts. Immunoprecipitation of normal human frontal cortex extract with a monoclonal antibody specific for parkin protein (FIGS. 9-11), followed by Western blotting with a polyclonal antibody against hSel-10, indicated that parkin and hSel-10 are associated (FIG. 1E). In contrast, immunoprecipitation of an age-matched, parkin-deficient ARPD frontal cortex extract, with a Parkin-specific monoclonal antibody, failed to co-purify hSel-10. Parkin antibody immunoprecipitation of normal human frontal cortex extract (FIG. 1E) or transfected HeLa cell lysates (data not shown) failed to co-purify any form of α-synuclein. Purified Flag-hSel-10, when added to mouse cortex extract, associated with endogenous brain parkin in pull-down assays, whereas purified Flag-β-TrCP failed to do so (FIG. 1F). As previously reported (Strohmaier et al., Human F-box protein hCdc4 targets cyclin E for proteolysis and is mutated in a breast cancer cell line. Nature, 413:316-22, 2001), tagged hSel-10 effectively pulled down cyclin E as well (FIG. 1F).

HSel-10 Potentiates Parkin Ubiquitin Ligase Activity

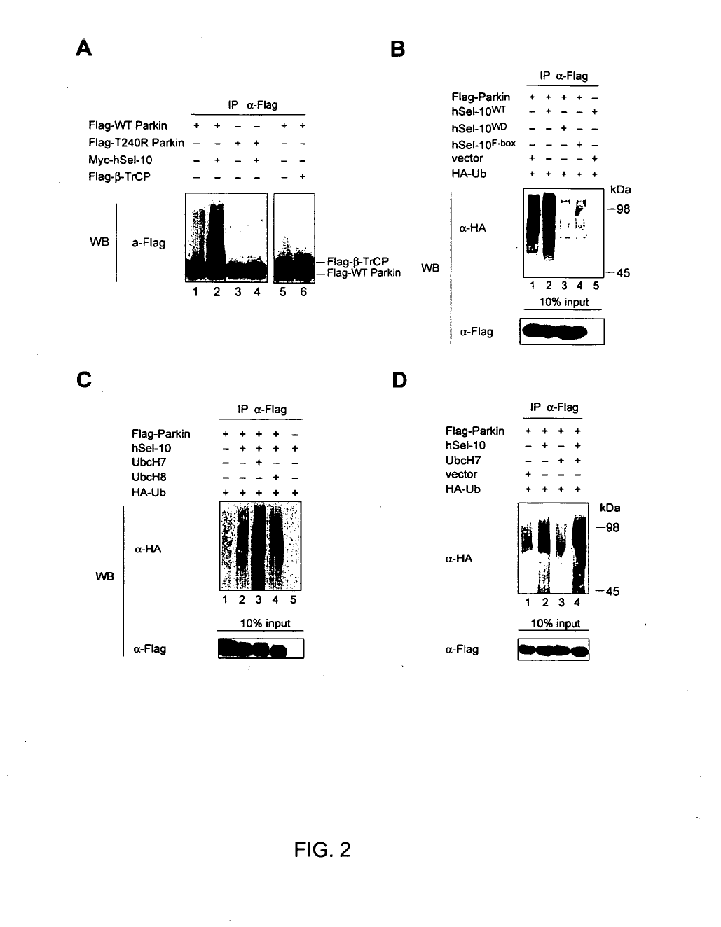

The inventors hypothesized that hSel-10 may be a component of a parkin-associated ubiquitin ligase complex, rather than a substrate. Consistent with this notion, the inventors did not observe parkin-dependent ubiquitination or proteolysis of hSel-10 (data not shown). Similar to several other RING-domain ubiquitin ligases, parkin auto-ubiquitinates (Zhang et al., Parkin functions as an E2-dependent ubiquitin-protein ligase and promotes the degradation of the synaptic vesicle-associated protein, CDCrel-1. Proc. Natl. Acad. Sci. USA, 97:13354-359, 2000). The inventors examined whether hSel-10 overexpression modifies parkin ubiquitin ligase activity. Expression vectors encoding Flag-tagged wild-type or T240R (ARPD mutant) parkin, as well as either hSel-10 or β-TrCP, were transfected, along with hemagglutinin-(HA)-tagged ubiquitin, into HeLa cells. Flag immunoprecipitation from cell lysates, and subsequent Western blotting for the Flag tag, revealed a high-molecular-weight smear in lysates from cells transfected with wild-type (FIG. 2A, lane 1), but not T240R mutant parkin (FIG. 2A, lane 3), consistent with auto-ubiquitination of parkin. Indeed, Western blots of Flag immunoprecipitates for HA-ubiquitin (FIG. 2B, lane 1) again demonstrated a high-molecular-weight smear in lysates from wild-type parkin-transfected cells, confirming that these species are ubiquitinated derivatives of parkin.

Overexpression of hSel-10 dramatically potentiated the ubiquitin ligase activity of wild-type (FIG. 2A, lane 2 and FIG. 2B, lane 2), but not T240R ARPD mutant, parkin (FIG. 2A, lane 4). In contrast, overexpression of β-TrCP (FIG. 2A, lane 6) failed to potentiate parkin ubiquitin ligase activity. As HeLa cells express endogenous hSel-10 (Koepp et al., Phosphorylation-dependent ubiquitination of cyclin E by the SCFFbw7 ubiquitin ligase. Science, 294:173-77, 2001), the basal level of parkin ubiquitination in untransfected HeLa cells may be due to endogenous hSel-10 or a related activity. Therefore, the inventors co-transfected tagged parkin and ubiquitin expression constructs, as above, along with deletion mutants of hSel-10 (WD-repeat domain alone (WD) and F-box domain alone (F-box)) (Strohmaier et al., Human F-box protein hCdc4 targets cyclin E for proteolysis and is mutated in a breast cancer cell line. Nature, 413:316-22, 2001) that are thought to function in a dominant-negative manner, and to bind to wild-type parkin (FIGS. 1D and 9-11). Indeed, overexpression of these hSel-10 mutants inhibited parkin-mediated ubiquitination, indicating that parkin ubiquitin ligase activity requires hSel-10 or a related activity in vivo (FIG. 2B, lanes 3 and 4).

The inventors further investigated whether the E2 ubiquitin-conjugating enzyme, UbcH7, functions in the above parkin ubiquitination assay by co-transfecting a UbcH7 expression construct. Consistent with the protein interaction data (FIG. 1A), the inventors found that overexpression of UbcH7 (FIG. 2C, lane 3), but not UbcH8 (FIG. 2C, lane 4) or Ubc6 (data not shown), increased parkin-mediated ubiquitination. Furthermore, the enhancement of parkin-mediated ubiquitination, by UbcH7 overexpression, required co-expression of hSel-10 (FIG. 2D, lanes 3 and 4). Thus, parkin functions cooperatively with hSel-10 and UbcH7.

A Parkin-HSel-10-Cullin-1 Complex

HSel-10 has been shown to function in cell-cycle regulation within a modular, multiprotein E3 ubiquitin ligase complex, termed the SCF^{hSel-10} complex (for Skp1, Cullin, and F-box) (Patton et al., Combinatorial control in ubiquitin-dependent proteolysis: don't Skp the F-box hypothesis. Trends Genet., 14:236-43, 1998; Skowyra et al., F-box proteins are receptors that recruit phosphorylated substrates to the SCF ubiquitin-ligase complex. Cell, 91: 9-19, 1997), that includes Rbx1, a RING domain protein (Kamura et al., Rbx1, a component of the VHL tumor suppressor complex and SCF ubiquitin ligase. Science, 284:657-61, 1999; Skowyra et al., Reconstitution of G1 cyclin ubiquitination with complexes containing SCFGrr1 and Rbx1. Science, 284:662-65, 1999). Therefore, the inventors speculated that other characterized SCF^{hSel-10} components might be present in the parkin-hSel-10 complex.

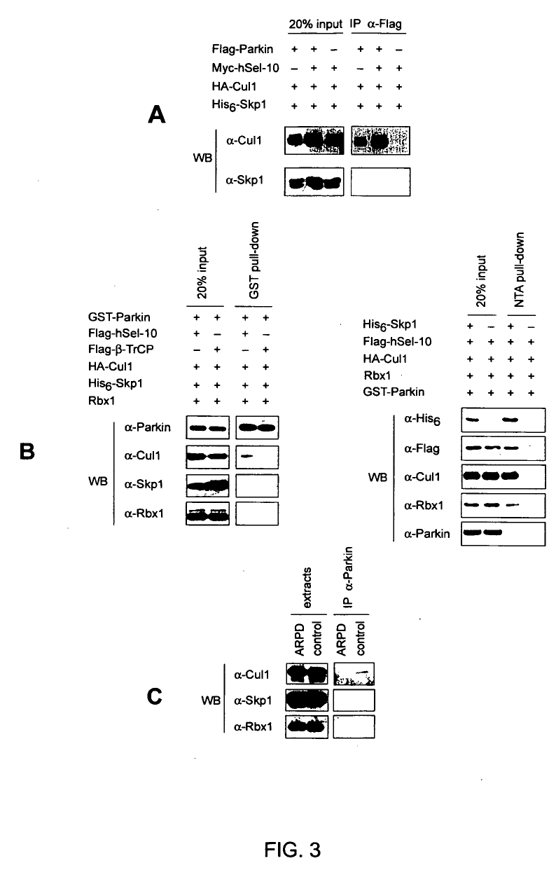

To investigate this possibility, the inventors co-expressed tagged parkin with tagged forms of Cul-1, Skp1, and Rbx1, in HeLa and insect cells. Subsequent pull-down assays revealed that parkin associates with Cul-1, but not Skp1 or Rbx1 (FIGS. 3A and 3B). The parkin-Cul-1 interaction appears to be modified by hSel-10, as the interaction is potentiated in HeLa cells that overexpress hSel-10 (FIG. 3A), and as parkin failed to associate with Cul-1 in insect cells in the absence of hSel-10 (FIG. 3B, left panel). Furthermore, the T240R ARPD parkin mutation attenuated the association of parkin with Cul-1 (FIGS. 9-11).

To investigate the relationship of the parkin-hSel-10 complex with the SCF^{hSel-10} complex, the inventors went on to perform a pull-down of His₆-Skp1 from the insect cell lysates. Tagged Skp1 co-purified Cul-1, hSel-10, and Rbx1 from insect cells (the SCF^{hSel-10} complex) (Koepp et al., Phosphorylation-dependent ubiquitination of cyclin E by the SCFFbw7 ubiquitin ligase. Science, 294:173-77, 2001; Strohmaier et al., Human F-box protein hCdc4 targets cyclin E for proteolysis and is mutated in a breast cancer cell line. Nature, 413:316-22, 2001), but not parkin (FIG. 3B, right panel). Flag-immunoprecipitation of hSel-10, as expected, co-precipitated parkin as well as all of the SCF components (FIGS. 9-11). Taken together, these data show that the parkin-hSel-10-Cul-1 complex is cooperative and distinct from the SCF^{hSel-10} complex.

The inventors next sought to confirm the presence of the parkin-hSel-10-Cul-1 complex in brain extracts. Immunoprecipitation of normal human frontal cortex brain extract (but not parkin-deficient ARPD frontal cortex extract), with a parkin-specific antibody, co-purified Cul-1, but not Skp1 or Rbx1 (FIG. 3C), consistent with the above data. Thus, parkin associates cooperatively with Cul-1 and hSel-10 in a novel complex that is distinct from SCF^{hSel-10}.

Ubiquitination of Cyclin E by Parkin

HSel-10 functions as an adaptor to recruit specific substrates for ubiquitination by the SCF^{hSel-10} complex, including cyclin E, a regulatory subunit of cyclin-dependent kinase 2 (CDK2) (Ekholm and Reed, Regulation of G(1) cyclin-dependent kinases in the mammalian cell cycle. Curr. Opin. Cell Biol., 12:676-84, 2000). Ubiquitination and degradation of phosphorylated cyclin E by the SCF hSel-1 complex underlies the regulation of cell-cycle entry into S phase. Interestingly, hSel-10 (Strohmaier et al., Human F-box protein hCdc4 targets cyclin E for proteolysis and is mutated in a breast cancer cell line. Nature, 413:316-22, 2001) is highly expressed in adult brain neurons, consistent with a role in post-mitotic cells. Of note, the accumulation of cyclins, including cyclin E, has been implicated in the regulation of apoptosis in post-mitotic neurons, as increased cyclin levels correlate with apoptosis (Verdaguer et al., Kainic acid-induced apoptosis in cerebellar granule neurons: an attempt at cell cycle re-entry. Neuroreport, 13:413-16, 2002), and cyclin-dependent kinase (CDK) inhibitors prevent such neuronal death (Copani et al., Activation of cell-cycle-associated proteins in neuronal death: a mandatory or dispensable path? Trends Neurosci., 24:25-31, 2001; Liu and Greene, Neuronal apoptosis at the G1/S cell cycle checkpoint. Cell Tissue Res., 305:217-28, 2001; Padmanabhan et al., Role of cell cycle regulatory proteins in cerebellar granule neuron apoptosis. J. Neurosci., 19:8747-56, 1999; Park et al., Cyclin-dependent kinases participate in death of neurons evoked by DNA-damaging agents. J. Cell Biol., 143:457-67, 1998). Furthermore, cyclin and CDK levels are increased in neurons in the course of several neurodegenerative disorders, such as PD (Chung et al., The role of the ubiquitin-proteasomal pathway in Parkinson's disease and other neurodegenerative disorders. Trends Neurosci., 24:S7-14, 2001; Husseman et al., Mitotic activation: a convergent mechanism for a cohort of neurodegenerative diseases. Neurobiol. Aging, 21:815-28, 2000).

The inventors hypothesized that hSel-10 may recruit cyclin E to the parkin-hSel-10-Cul-1 complex in post-mitotic neurons in a manner that is analogous to its role as an adaptor in SCF^{hSel-10}. Because hSel-10 is known to bind directly to cyclin E, it may recruit cyclin E and other substrates for modification by a parkin-hSel-10-Cul-1 E3 complex. Of note, cullins, including Cul-1, have been implicated directly in the ubiquitination of cyclin E (Dealy et al., Loss of Cull results in early embryonic lethality and dysregulation of cyclin E. Nat. Genet., 23:245-48, 1999; Singer et al., Cullin-3 targets cyclin E for ubiquitination and controls S phase in mammalian cells. Genes Dev., 13:2375-87, 1999). Thus, the inventors further hypothesized that the parkin-hSel-10-Cul-1 E3 complex may target cyclin E, and that parkin-associated ARPD may lead to toxic accumulation of this substrate.

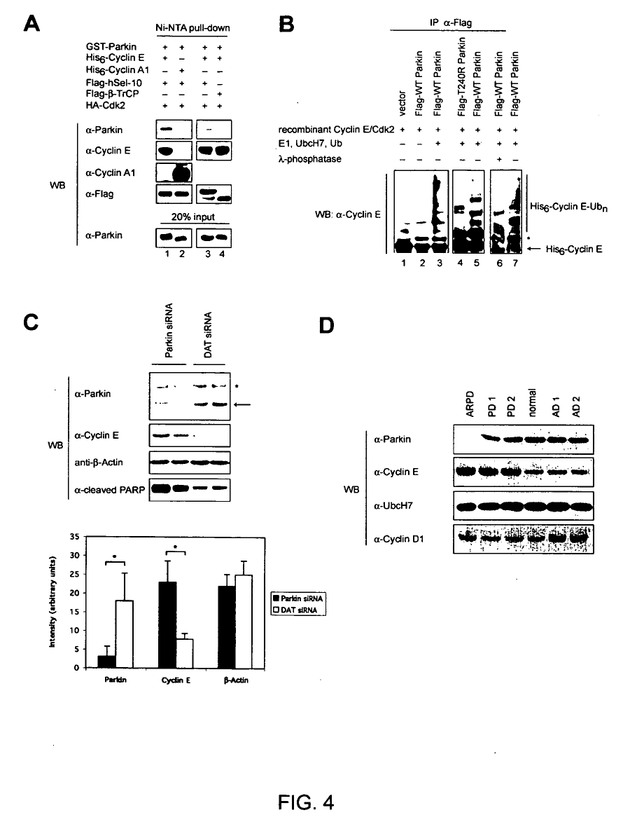

The inventors first tested the hypothesis that hSel-10 could recruit cyclin E to a parkin-associated complex. Insect cells were infected with baculoviruses encoding GST-parkin, Flag-hSel-10 (or Flag-β-TrCP), and His₆-cyclin E (or His₆-cyclin A1), and HA-CDK2 (which stabilizes phosphorylated forms of cyclin E that interact with hSel-10 (Clurman et al., Turnover of cyclin E by the ubiquitin-proteasome pathway is regulated by cdk2 binding and cyclin phosphorylation. Genes Dev., 10:1979-90, 1996). Cell lysates were subsequently analyzed by pull-down assays and Western blotting. These studies confirmed that cyclin E (FIG. 4A, lane 1), but not cyclin A1 (lane 2), could be recruited to a parkin-associated complex by hSel-10 (FIG. 4A, lanes 1 and 3), but not by β-TrCP (FIG. 4A, lane 4).

The inventors also sought to determine whether a parkin-associated complex is able to ubiquitinate cyclin E substrate in vitro. The inventors found that a Flag-immunoprecipitated, wild-type parkin-associated complex (from lysates of HeLa cells transfected with Flag-parkin) could modify recombinant cyclin E/CDK2 substrate in the presence of other ubiquitination components in vitro (FIG. 4B, lanes 3, 5 and 7), whereas T240R ARPD mutant parkin complex failed to do so (FIG. 4B, lane 4). Furthermore, cyclin E ubiquitination appeared to be phosphorylation-dependent, as pre-treatment of the substrate with λ-phosphatase inhibited the modification (FIG. 4B, lanes 6 and 7).

Parkin Deficiency Potentiates the Accumulation of Cyclin E

The inventors hypothesized that parkin deficiency would potentiate the accumulation of cyclin E in primary neurons. Previous studies have indicated that primary neuronal cultures accumulate cyclin E in response to the glutamatergic excitotoxin, kainate (Padmanabhan et al., Role of cell cycle regulatory proteins in cerebellar granule neuron apoptosis. J. Neurosci., 19:8747-56, 1999; Verdaguer et al., Kainic acid-induced apoptosis in cerebellar granule neurons: an attempt at cell cycle re-entry. Neuroreport, 13:413-16, 2002), and the inventors confirmed this to be the case for primary cortical, cerebellar granule, and midbrain neuron cultures (see below, and data not shown).

To investigate the role of parkin in the accumulation of cyclin E, primary cortical cultures (prepared from embryonic day 16.5 (E16.5) embryos) were transfected with 25 nM parkin-specific or control (dopamine transporter-specific) short interfering RNAs (siRNAs) (Elbashir et al., Duplexes of 21-nucleotide RNAs mediate RNA interference in cultured mammalian cells. Nature, 411:494-98, 2001), and subsequently treated with kainate. Western-blot analysis of lysates from cultures transfected with control siRNA revealed readily detectable parkin protein expression (FIG. 4C), whereas lysates from parkin siRNA-transfected cultures displayed significantly reduced parkin expression. As predicted, parkin-deficient cultures displayed increased accumulation of cyclin E (FIG. 4C). Furthermore, such cultures displayed accumulation of cleaved poly (ADP-ribose) polymerase (cleaved-PARP), a marker of apoptosis.

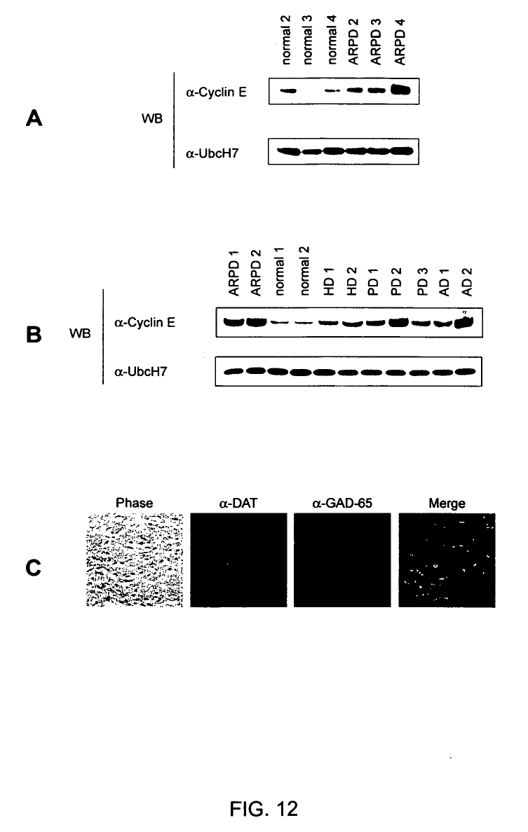

Parkin deficiency leads to neuronal loss in ARPD, and PD has been associated with apoptotic neuronal death (Burke and Kholodilov, Programmed cell death: does it play a role in Parkinson's disease? Ann. Neurol., 44:S126-33, 1998). Therefore, the inventors next investigated whether cyclin E is accumulated in extracts from Parkin-deficient human ARPD brain. Western blotting with a specific antibody demonstrated accumulation of cyclin E in substantia nigra from ARPD brain tissue extract, relative to age-matched control extract (FIG. 4D). In contrast, no accumulation was observed for three other proteins: cyclin D1, UbcH7, and α-synuclein (FIG. 4D, and data not shown). Similar accumulation of cyclin E was observed in frontal cortex extract from ARPD brain, and in cortical extracts from three independent ARPD cases, relative to three normal controls (FIG. 12).

Analysis of cyclin E mRNA by quantitative real-time PCR indicated that the accumulation of cyclin E protein was not accounted for by differences in cyclin E mRNA transcript levels (FIGS. 9-11). Analysis of cyclin E protein accumulation in substantia nigra extracts from sporadic PD and AD cases similarly demonstrated the accumulation of cyclin E in sporadic PD, but not sporadic AD, nigral extracts (FIG. 4D), consistent with the notion that cyclin E accumulation may be relevant to sporadic PD, as well as parkin-associated ARPD. Finally, analysis of frontal cortex extracts from sporadic PD (relative to AD, Huntington's disease, and normal control) brains revealed a variable degree of cyclin E accumulation (FIG. 12).

Parkin Overexpression Inhibits the Accumulation of Cyclin E

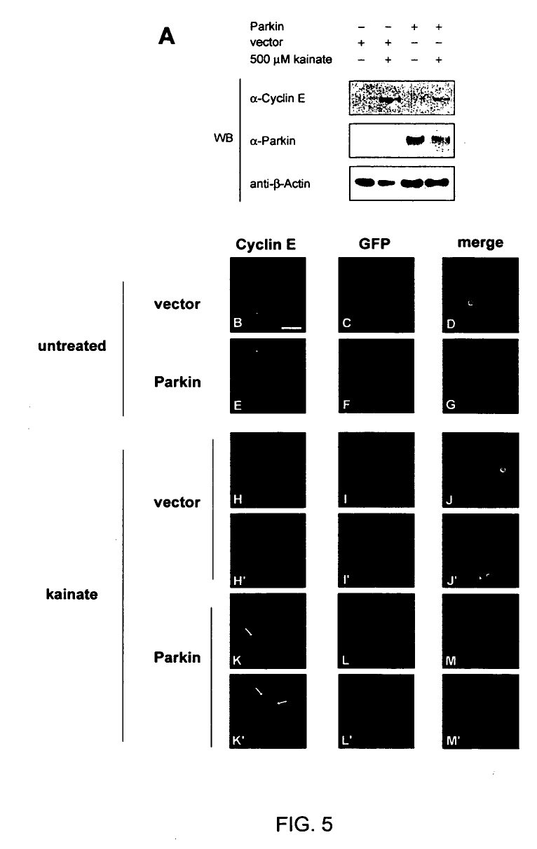

The inventors investigated the effect of parkin overexpression on kainate-induced apoptosis of cultured cerebellar granule cells, as these cells are readily purified to near homogeneity (Scheiffele et al., Neuroligin expressed in nonneuronal cells triggers presynaptic development in contacting axons. Cell, 101:657-69, 2000) and appear to be devoid of endogenous parkin expression (FIG. 5A). Furthermore, cyclin E has been shown to accumulate with apoptosis in such cultures (Padmanabhan et al., Role of cell cycle regulatory proteins in cerebellar granule neuron apoptosis. J. Neurosci., 19:8747-56, 1999; Verdaguer et al., Kainic acid-induced apoptosis in cerebellar granule neurons: an attempt at cell cycle re-entry. Neuroreport, 13:413-16, 2002).

Granule neurons transfected with a bicistronic expression plasmid encoding wild-type parkin or vector alone (along with green fluorescent protein (GFP)) were treated with kainate (500 μM for 24 h). Subsequently, cultures were analyzed by Western blotting or immunofluorescence microscopy. As previously described, kainate treatment led to the accumulation of cyclin E in granule cell cultures (FIG. 5, panels A, B, E, H, and H′). Furthermore, overexpression of parkin significantly attenuated the accumulation of cyclin E (FIG. 5, panels H, H′, K, and K′). Analysis of cyclin E mRNA by quantitative real-time PCR indicated that the accumulation of cyclin E protein was not accounted for by differences in cyclin E mRNA (FIGS. 9-11).

Parkin Overexpression Protects Post-Mitotic Neurons from Kainate-Mediated Excitotoxicity

Cell-cycle regulatory proteins have been implicated in the apoptotic death of post-mitotic cells. Cyclins, including cyclin E, accumulate in post-mitotic cells destined for apoptosis, whereas inhibitors of cyclin-dependent kinases block apoptosis (Copani et al., Activation of cell-cycle-associated proteins in neuronal death: a mandatory or dispensable path? Trends Neurosci., 24:25-31, 2001; Liu and Greene, Neuronal apoptosis at the G1/S cell cycle checkpoint. Cell Tissue Res., 305:217-28, 2001; Padmanabhan et al., Role of cell cycle regulatory proteins in cerebellar granule neuron apoptosis. J. Neurosci., 19:8747-56, 1999; Park et al., Cyclin-dependent kinases participate in death of neurons evoked by DNA-damaging agents. J. Cell Biol., 143:457-67, 1998). In contrast to cyclin E regulation at the G1/S cell-cycle checkpoint, little is known about the regulation of cyclin E accumulation in post-mitotic cells. Based on the above data, the inventors hypothesized that parkin may play a role in the regulation of cyclin E in the context of neuronal apoptosis, and that parkin overexpression would protect post-mitotic neurons from cell death.

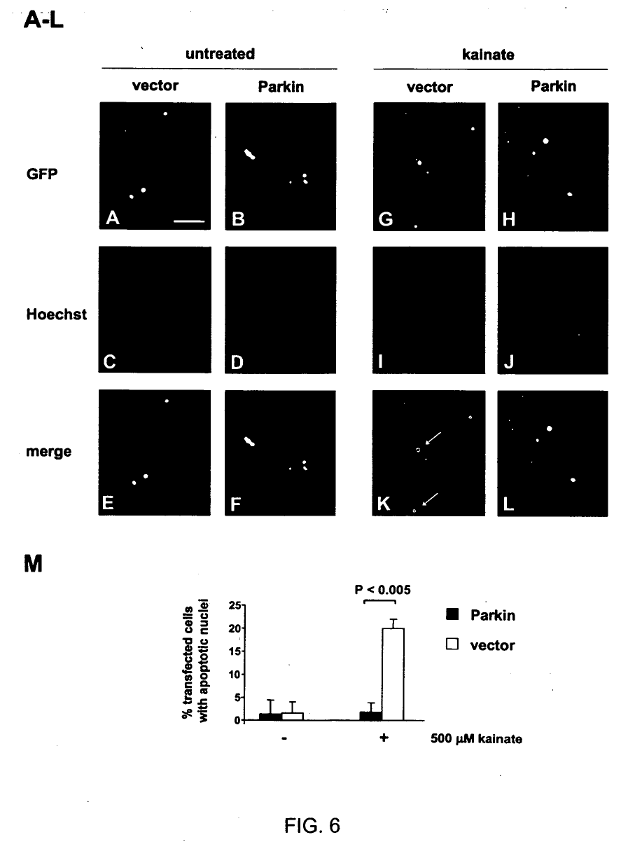

As described above, cyclin E is upregulated in the course of kainate-induced apoptosis of cultured cerebellar granule cells, and overexpression of parkin attenuates the accumulation of cyclin E. To investigate whether parkin overexpression would protect these cells from apoptosis, granule neurons were transfected and treated with kainate, as above. Apoptosis was quantified by visualization of condensed nuclei using Hoechst staining and fluorescence microscopy (FIG. 6). Neuronal cultures transfected with parkin showed significantly fewer apoptotic nuclei than vector-transfected cells (FIG. 6). Thus, parkin overexpression can protect post-mitotic neurons from toxin-mediated apoptosis, and this may be a consequence of inhibiting cyclin E accumulation.

Parkin and Dopamine Neuron Survival

ARPD and sporadic PD lead to the relatively specific loss of dopamine neurons, although additional neuronal populations are affected to a variable extent. Furthermore, glutamate excitotoxicity has been implicated as a potential mechanism for dopamine neuron loss in PD ((Lang and Lozano, Parkinson's disease. First of two parts. N. Engl. J. Med., 339:1044-53, 1998; Olanow and Tatton, Etiology and pathogenesis of Parkinson's disease. Annu. Rev. Neurosci., 22 :123-44, 1999). As described above, parkin deficiency leads to increased cyclin E accumulation and the expression of apoptotic markers in the context of an excitotoxic insult to primary neurons, while parkin over-expression protects primary neurons. The inventors sought to extend these studies to primary dopamine neuron cultures.

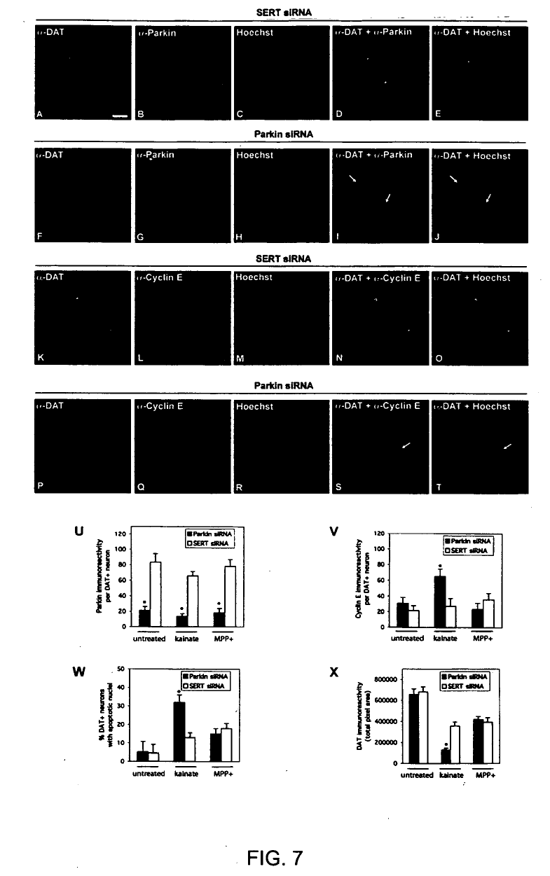

Embryonic day 13.5 (E13.5) primary culture midbrain dopamine neurons, identified by immunohistochemical staining for the dopamine transporter (DAT) (Nirenberg et al., The dopamine transporter is localized to dendritic and axonal plasma membranes of nigrostriatal dopaminergic neurons. J. Neurosci., 16:436-47, 1996), were transfected with parkin siRNA (FIG. 7, panels F-J and P-T) or control siRNA (FIG. 7, panels A-E and K-O), and subsequently exposed to kainate, as above. As predicted, parkin “knockdown” dopamine neurons displayed increased accumulation of cyclin E (FIG. 7, panel S) and increased apoptosis (FIG. 7, panels J and T), as compared with siRNA-treated cells. Furthermore, parkin-siRNA-treated midbrain cultures displayed decreased DAT immunoreactivity in cell bodies and processes in the presence of kainate, as compared with control siRNA-treated cells (FIG. 7, panel I), consistent with the increased sensitivity to kainate excitotoxicity. Parkin siRNA treatment alone, in the absence of kainate, failed to alter cyclin E or DAT immunoreactivity (FIG. 7, panels U-X); thus, parkin “knockdown” is not directly toxic, but appears to sensitize neurons to kainate excitotoxicity.

Parkin siRNA treatment failed to alter dopamine neuron sensitivity to 1-Methyl-4-phenylpyridinium (MPP⁺; 10 μM) at a toxin dose that (in control siRNA-treated cultures) led to a reduction in DAT immunoreactivity comparable to the kainate treatment (FIG. 7, panel X). The inventors further investigated the effect of parkin “knockdown” on the kainate sensitivity of DAT-negative neurons in midbrain cultures, which are primarily GABAergic (greater than 90%) (FIG. 12, in order to determine the specificity of parkin action. Parkin siRNA did sensitize DAT-negative neurons to kainate toxicity, but to a significantly lesser extent than it did the DAT-positive population, with respect to cyclin E induction and apoptosis (p<0.05 for both measures; FIGS. 9-11). Thus, parkin deficiency appears to preferentially sensitize midbrain dopamine neurons to kainate excitotoxicity.

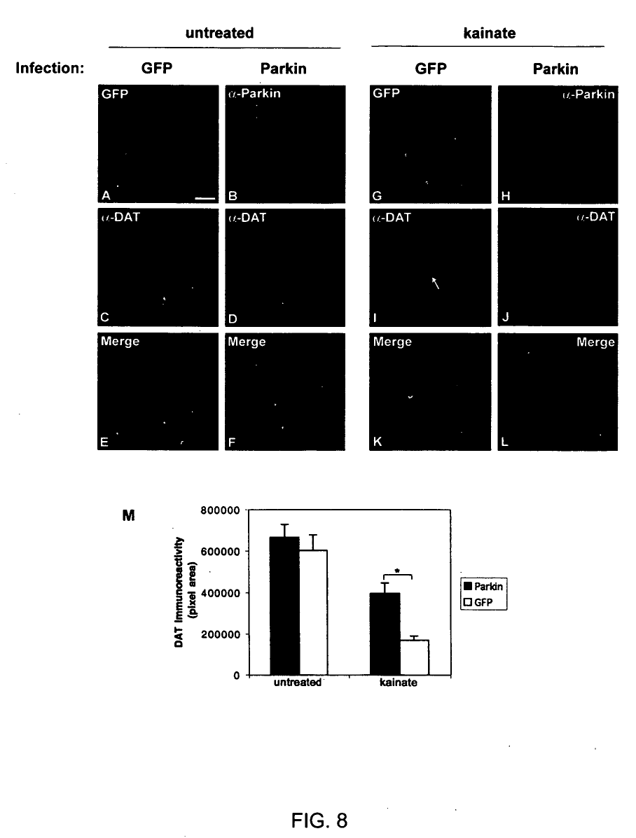

Overexpression of parkin using a lentiviral vector (in E13.5 midbrain dopamine neuron cultures) conferred robust protection of dopaminergic cell bodies and processes from 250 μM kainate, as quantified by DAT immunohistochemistry (FIG. 8), as compared to control lentivirus). Both parkin and control viruses infected over 90% of DAT-positive neurons (FIGS. 9-11, and data not shown). Parkin overexpression did not appear to alter sensitivity to MPP⁺ (FIGS. 9-11). Furthermore, parkin overexpression did not alter DAT immunoreactivity in primary midbrain neuron cultures in the absence of toxin (FIG. 8, panel M).

As demonstrated above, parkin associates with hSel-10 and Cul-1 in a novel ubiquitin ligase complex. The parkin ubiquitin ligase complex functions in parkin auto-ubiquitination and in hetero-ubiquitination of cyclin E. The inventors also present evidence that parkin does appear to regulate cyclin E in the course of neuronal apoptosis, in dopamine neurons, and in ARPD tissue. The inventors hypothesize that, in addition to cyclin E, there are additional targets of parkin ubiquitination, as other characterized RING-finger-associated E3 complexes appear to target multiple diverse substrates (Joazeiro and Weissman, RING finger proteins: mediators of ubiquitin ligase activity. Cell, 102:549-52, 2000). For example, genetic and biochemical evidence implicate hSel-10 in the ubiquitination of Notch4 (Wu et al., SEL-10 is an inhibitor of Notch signaling that targets Notch for ubiquitin-mediated protein degradation. Mol. Cell Biol., 21:7403-15, 2001) and presenilin (Wu et al., Evidence for functional and physical association between Caenorhabditis elegans SEL-10, a Cdc4p-related protein, and SEL-12 presenilin. Proc. Natl. Acad. Sci. USA, 95:15787-791, 1998), the latter of which is mutated in autosomal dominant forms of Alzheimer's disease. Thus, these represent additional candidates for activity of the parkin-associated complex.

SCF complexes are modular: for instance, Skp1 can interact with several F-box adaptor proteins, thereby generating functional diversity. It is of interest to determine whether parkin associates with adaptor proteins other than hSel-10 (although the inventors failed to detect an interaction with other F-box/WD-repeat proteins in the foregoing Examples), as such complexes would likely display different substrate specificities. This may explain the diverse targets that have been reported for parkin, including CDCrel-1 (Zhang et al., Parkin functions as an E2-dependent ubiquitin-protein ligase and promotes the degradation of the synaptic vesicle-associated protein, CDCrel-1. Proc. Natl. Acad. Sci. USA, 97: 13354-359, 2000), synphilin-1 (Chung et al., Parkin ubiquitinates the alpha-synuclein-interacting protein, synphilin-1: implications for Lewy-body formation in Parkinson disease. Nat. Med., 7:1144-50, 2001), PAEL-R (Imai et al., An unfolded putative transmembrane polypeptide, which can lead to endoplasmic reticulum stress, is a substrate of Parkin. Cell, 105:891-02, 2001), and a modified form of α-synuclein (αSp22) (Shimura et al., Ubiquitination of a new form of alpha-synuclein by parkin from human brain: implications for Parkinson's disease. Science, 293:263-69, 2001). Recently, parkin has been reported to form a complex with the heat-shock protein, Hsp70, as well as CHIP, an Hsp70-associated protein with E3 activity, in SH-SY5Y cells that overexpress parkin (Imai et al., CHIP is associated with parkin, a gene responsible for familial Parkinson's disease, and enhances its ubiquitin ligase activity. Mol. Cell., 10:55-67, 2002). It remains to be determined whether SCF-like components play a role in this complex.

The inventors' data support the notion that there is both redundancy and specificity in the regulation of cyclin E (Koepp et al., Phosphorylation-dependent ubiquitination of cyclin E by the SCFFbw7 ubiquitin ligase. Science, 294:173-77, 2001; Winston et al., Culprits in the degradation of cyclin E apprehended. Genes Dev., 13:2751-57, 1999). Although cyclin E accumulation has been noted in the context of several apoptosis model systems, the mechanism of regulation of cyclin E in neuronal apoptosis has not previously been investigated. The data presented here suggest that parkin regulates the degradation of cyclin E in the context of neuronal apoptosis. Furthermore, as the parkin ubiquitin ligase complex targets cyclin E, this effect is likely to be direct.

Cyclin regulation, specifically regulation of cyclin E, has previously been implicated in kainate-excitotoxin-induced neuronal apoptosis (Padmanabhan et al., Role of cell cycle regulatory proteins in cerebellar granule neuron apoptosis. J. Neurosci., 19:8747-56, 1999; Verdaguer et al., Kainic acid-induced apoptosis in cerebellar granule neurons: an attempt at cell cycle re-entry. Neuroreport, 13:413-16, 2002). The inventors have shown herein that parkin overexpression protects dopamine neurons from kainate-mediated apoptosis, that parkin “knockdown” (using siRNA) sensitizes dopamine neurons to such excitotoxicity, and that this correlates with the accumulation of cyclin E. The inventors note that glutamate excitotoxicity has been implicated in sporadic PD (Olanow and Tatton, Etiology and pathogenesis of Parkinson's disease. Annu. Rev. Neurosci., 22:123-44, 1999; Schulz et al., The role of mitochondrial dysfunction and neuronal nitric oxide in animal models of neurodegenerative diseases. Mol. Cell. Biochem., 174:193-97, 1997), and that excitatory input ablation appears to protect dopamine neurons (Klein et al., The harlequin mouse mutation downregulates apoptosis-inducing factor. Nature, 419:367-74, 2002; Olanow and Tatton, Etiology and pathogenesis of Parkinson's disease. Annu. Rev. Neurosci., 22:123-44, 1999; Raina et al., Cyclin' toward dementia: cell cycle abnormalities and abortive oncogenesis in Alzheimer disease. J. Neurosci. Res., 61:128-33, 2000; Takada et al., Protection against dopaminergic nigrostriatal cell death by excitatory input ablation. Eur. J. Neurosci., 12:1771-80, 2000). Parkin deficiency appeared to preferentially sensitize midbrain dopamine neurons (versus midbrain GABAergic neurons) to kainate excitotoxicity, as may be the case in parkin-associated ARPD. Parkin overexpression did not appear to protect cultured primary dopamine neurons from MPP⁺ toxicity, and parkin knockdown (with siRNA) did not appear to sensitize dopamine neurons to MPP⁺ (at least under the conditions used here). These data suggest that different mechanisms may underlie kainate- and MPP⁺-mediated toxicity, and, indeed, it has been reported that MPP⁺ treatment induces non-apoptotic death in neuronal midbrain cultures (Lotharius et al., Distinct mechanisms underlie neurotoxin-mediated cell death in cultured dopaminergic neurons. J. Neurosci., 19:1284-93, 1999), in contrast with kainate. In a recent report (Petrucelli et al., Parkin protects against the toxicity associated with mutant alpha-synuclein: proteasome dysfunction selectively affects catecholaminergic neurons. Neuron, 36: 1007-19, 2002), parkin overexpression was found to protect primary midbrain catecholaminergic neurons from non-apoptotic death associated with the overexpression of mutant α-synuclein or proteasomal inhibition. The molecular mechanism of this protection appears to differ from that underlying the protection of primary neurons from excitotoxin-mediated apoptotic death, as described herein.

Finally, the protective role of parkin overexpression suggests a treatment approach for PD and other diseases that relate to glutamate excitotoxicity. Thus, an understanding of the parkin-associated ubiquitin ligase complex described herein, and its mechanism of action, can lead to novel diagnostic and therapeutic tools.

### Example 2

Generation of DJ-1 Deficient ES Cells

To investigate the normal cellular function of DJ-1 and the pathogenic mechanism of the PD mutations, the inventors generated cells deficient in DJ-1. A murine embryonic stem (ES) cell clone, F063A04, that harbors a retroviral insertion at the DJ-1 locus was obtained through the German Gene Trap Consortium (Tikus web site) (Floss and Wurst 2002). The pT1ATGβgeo gene trap vector is present between exons 6 and 7 of the murine DJ-1 gene, as determined by cDNA sequencing of trapped transcripts and genomic analysis (FIG. 1A). This integration is predicted to disrupt the normal splicing of DJ-1, leading to the generation of a truncated protein that lacks the carboxy-terminal domain required for dimerization and stability (data not shown). Of note, a mutation that encodes a similarly truncated protein (at the human DJ-1 exon 7 splice acceptor) has been described in a patient with early-onset PD (Hague et al. 2003).

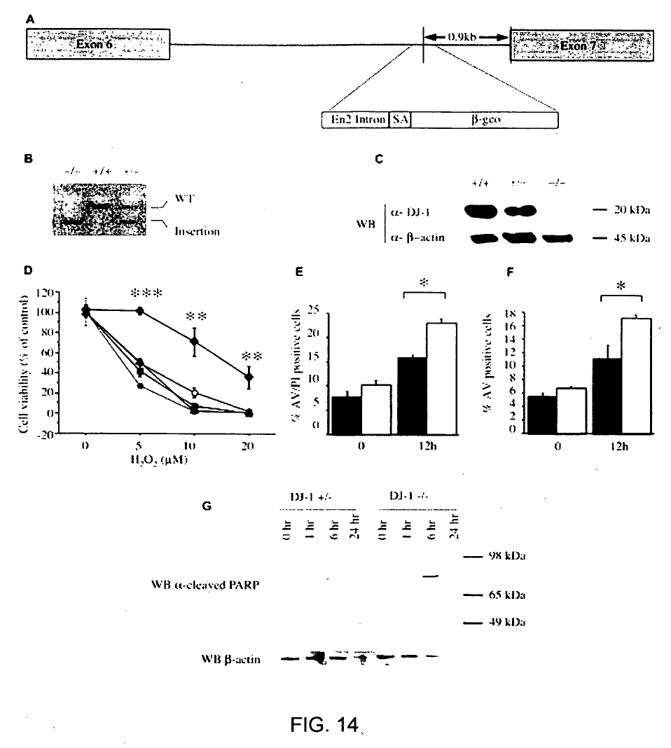

To select for ES subclones homozygous for the trapped DJ-1 allele, clone F063A04 was exposed to high-dose G418 (4 mg/ml) (Mizushima et al. 2001). Several homozygous mutant ES subclones (that have undergone gene conversion at the DJ-1 locus) were identified by Southern blotting (FIG. 14B). To confirm that the trapped allele leads to the loss of wild-type DJ-1 protein, cell lysates from ‘knockout’ homozygous clones as well as the parental heterozygous clone were analyzed by Western blotting using polyclonal antibodies to the amino terminal region of DJ-1 (amino acids 64-82) or full-length protein (data not shown). Neither full-length nor truncated DJ-1 protein products were detected in ‘knockout’ clones (FIG. 14C), consistent with instability of the predicted truncated DJ-1 product, and no full-length DJ-1 RNA was detected in the mutant cultures. In contrast, heterozygous and wild-type (control) ES cells express high levels of DJ-1. Initial phenotypic analysis of DJ-1-deficient ES subclones indicated that DJ-1 is non-essential for the growth rate of ES cells in culture, consistent with the viability of humans with homozygous DJ-1 mutation.

DJ-1 Protects Cells from Oxidative Stress and Proteasomal Inhibition

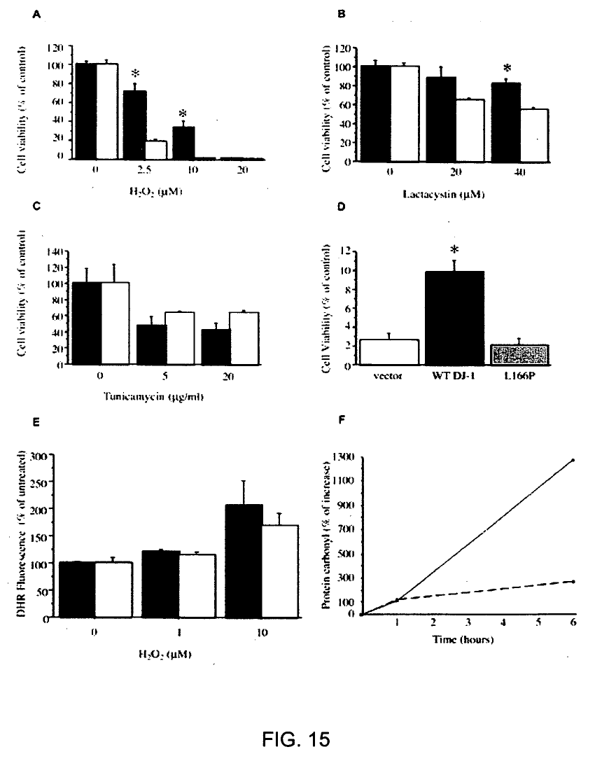

DJ-1 has been hypothesized to function in the cellular response to oxidative stress. To investigate the role of DJ-1 in the oxidative stress response in vivo, DJ-1-deficient homozygous mutant (‘knockout’) cells and DJ-1 heterozygote (‘control’) ES cell clones were analyzed for cell viability in the context of increasing concentrations of H₂O₂. Heterozygous cells were used as controls because the ‘knockout’ subclones were derived from these. Cell viability was initially determined by MTT assay in triplicate (Fezoui et al. 2000). Exposure to H₂O₂ led to significantly greater toxicity in DJ-1 deficient cells; similar results were obtained with multiple DJ-1 deficient subclones in independent experiments (FIGS. 14D and 15A). Untreated heterozygous and homozygous cells displayed comparable viability in the MTT assay in the absence of toxin. Consistent with the MTT assay, fluorescence activated cell sorting (FACS) analysis of cells stained with Annexin V (AV) and propidium iodide (PI) revealed increased cell death of knockout cells (relative to heterozygous cells) in the context of H₂O₂ exposure (FIG. 14E). The increase in AV-positive cells implicated an apoptotic mechanism of cell death (FIG. 14F). Furthermore, in the context of H₂O₂, knockout cells displayed potentiated cleavage of Poly(ADP-ribose)polymerase-1 (PARP) in a pattern indicative of an apoptotic death program (Gobeil et al. 2001) (FIG. 14G).

Additional toxin exposure studies demonstrated that DJ-1 deficient cells were sensitized to the proteasomal inhibitor lactacystin (FIG. 15B), as well as copper, which catalyzes the production of ROS. The inventors did not observe altered sensitivity to several other toxins including tunicamycin (an inducer of the unfolded protein response in the endoplasmic reticulum; FIG. 15C), staurosporine (a general kinase inhibitor that induces apoptosis), or cycloheximide (an inhibitor of protein translation; data not shown).

Wild-Type but not PD-Associated L166P Mutant DJ-1 Protects Cells from Oxidative Stress

To confirm that altered sensitivity to oxidative stress is a consequence of the loss of DJ-1, ‘rescue’ experiments by overexpressing wild-type or mutant human DJ-1 in ‘knockout’ ES cells were performed. Plasmids encoding human wild-type DJ-1, PD-associated L166P mutant DJ-1, or vector alone, were transiently transfected into DJ-1 deficient clones, and these were subsequently assayed for sensitivity to H₂O₂ using the MTT viability assay. DJ-1 deficient cells transfected with a vector encoding wild-type human DJ-1 were effectively ‘rescued’ in terms of viability in the presence of H₂O₂ (FIG. 15D); Thus, viability in ‘rescued’ knockout cells mimicked the viability of control (heterozygous) cells in the context of H₂O₂ treatment (FIGS. 15A, D). In contrast, transfection of a vector encoding the PD-associated L166P mutant DJ-1 did not significantly alter the viability of H₂O₂-treated knockout cells. Baseline cell viability in the absence of toxin exposure was not altered by DJ-1 overexpression, and Western blotting of lysates from transfected cells with an antibody specific to human DJ-1 demonstrated that transfected human wild-type and L166P mutant DJ-1 accumulated comparably.

DJ-1 Deficiency does not Alter the H₂O₂-Induced Intracellular ROS Burst

The inventors hypothesized that DJ-1 either alters the initial accumulation of intracellular ROS in response to H₂O₂ exposure, or alternatively that DJ-1 functions downstream of the ROS burst and protects cells from consequent damage. Therefore, the inventors quantified the accumulation of ROS in response to H₂O₂ treatment in mutant and heterozygous control cells using the ROS-sensitive fluorescent indicator dye dihydrorhodamine-123 (DHR) and FACS analysis. Initial ROS accumulation (at 15 minutes after stimulation) appeared unaltered in DJ-1 deficient cells in comparison to control heterozygous cells (FIG. 15E). Consistent with this, accumulation of protein carbonyls, an index of oxidative damage to proteins (Sherer et al. 2002), appeared normal initially (at 1 hour after toxin exposure; FIG. 15F). However at 6 hours after toxin exposure, a point at which knockout cells already display increased apoptosis (as determined by PARP cleavage; FIG. 14G), protein carbonyl accumulation was robustly increased in the DJ-1 deficient cells. These data suggest that initial ROS accumulation is not altered by DJ-1 deficiency, but that the mutant cells are unable to appropriately cope with the consequent damage. Consistent with this, antioxidant or peroxiredoxin activity with purified DJ-1 protein in vitro was not detected (Shendelman et al., S.R.B. and A.A., data not shown).

DJ-1 is Required for Survival of ES-Derived Dopamine Neurons

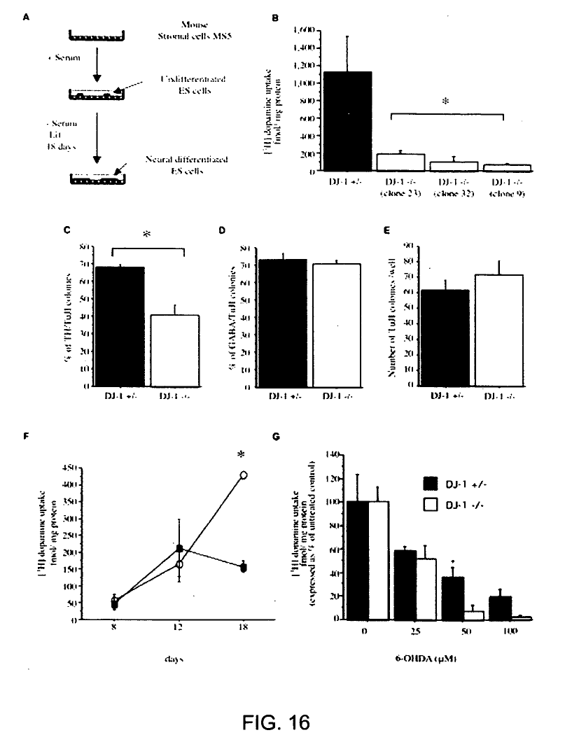

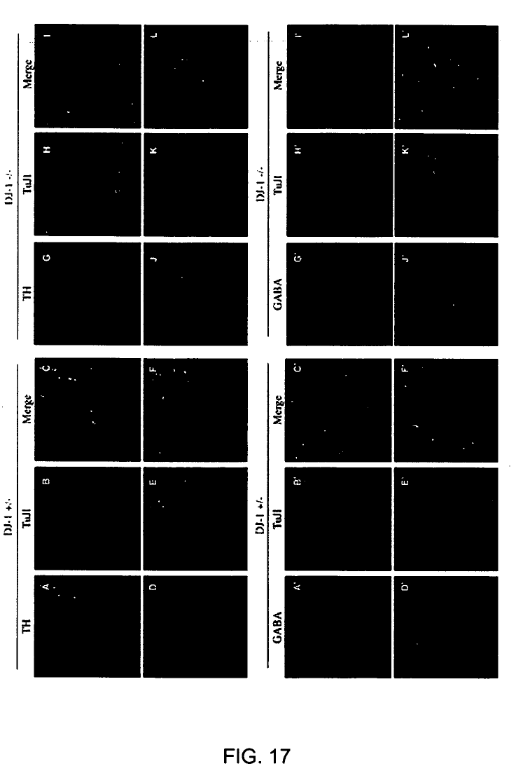

Several methods have been established for the differentiation of ES cells into dopamine neurons (DN) in vitro (Morizane et al. 2002). To extend this analysis of DJ-1 function to DNs, the inventors differentiated DJ-1-deficient ES cells or control heterozygous cells into DNs in vitro by co-culture with stromal cell-derived inducing activity (SDIA; FIG. 16A) (Morizane et al. 2002; Barberi et al. 2003). Dopamine neurons were quantified by immunohistochemistry for tyrosine hydroxylase (TH; a marker for dopamine neurons and other catecholaminergic cells), or by analysis of dopamine transporter uptake activity (a quantitative dopamine neuron marker) (Han et al. 2003). Production of dopamine neurons appeared to be significantly reduced in DJ-1-deficient ES cell cultures relative to parental heterozygous cultures at 18 days in vitro as determined both by dopamine uptake and TH immunoreactivity (DIV; FIGS. 16B and 16C, and 17A-L). In contrast, general neuronal production did not appear altered in this assay in terms of the post-mitotic neuronal marker Tuj1 (FIGS. 16E and 17A-L′), and other neuronal subtypes appeared normal, including GABAergic (FIGS. 16D and 17A′-L′) and motor neurons (HB9-positive). To investigate whether the reduction in dopamine neurons in DJ-1 deficient cultures is due to defective generation or survival, a time course analysis was performed. The inventors found that at early time points (8 and 12 DIV) dopamine uptake activity was comparable in wild-type and DJ-1 deficient cultures, whereas subsequently the DJ-1 deficient cultures appeared defective (FIG. 16F). Consistent with this, intracellular dopamine accumulation (as quantified using high performance liquid chromatography; HPLC) was significantly reduced in DJ-1 deficient cultures (6.4+/−1.5 ng dopamine/mg protein) relative to control heterozygous cultures (66.0+/−17.4 ng/mg) at 35 DIV. These data strongly suggest that DJ-1 deficiency leads to loss of dopamine neurons, rather than simply to downregulation of cell marker expression.

The inventors hypothesized that DJ-1 deficient dopamine neurons may be sensitized to oxidative stress, akin to DJ-1 deficient undifferentiated cells. To test this, dopamine neuron cultures from DJ-1-deficient or heterozygous control ES cultures at 9 DIV were exposed to oxidative stress in the form of 6-hydroxydopamine (6-OHDA), a dopamine neuron-specific toxin that enters dopamine neurons through the dopamine transporter and leads to oxidative stress and apoptotic death (Dauer and Przedborski 2003). DJ-1 deficient dopamine neurons displayed an increased sensitivity to oxidative stress in this assay (FIG. 16G). Post-hoc analysis of the data indicates that the difference among genotypes is maximal at an intermediate dose of toxin (50 μM); at the highest dose of 6-OHDA employed (100 μM) the difference is lessened, indicating that DJ-1-mediated protection is limited. Although a role for DJ-1 in the late stage differentiation of dopamine neurons cannot be excluded, these data suggest that DJ-1 deficiency leads to reduced dopamine neuron survival and predisposes these cells to endogenous and exogenous toxic insults.

RNAi ‘Knockdown’ of DJ-1 in Midbrain Embryonic Dopamine Neurons Leads to Increased Sensitivity to Oxidative Stress

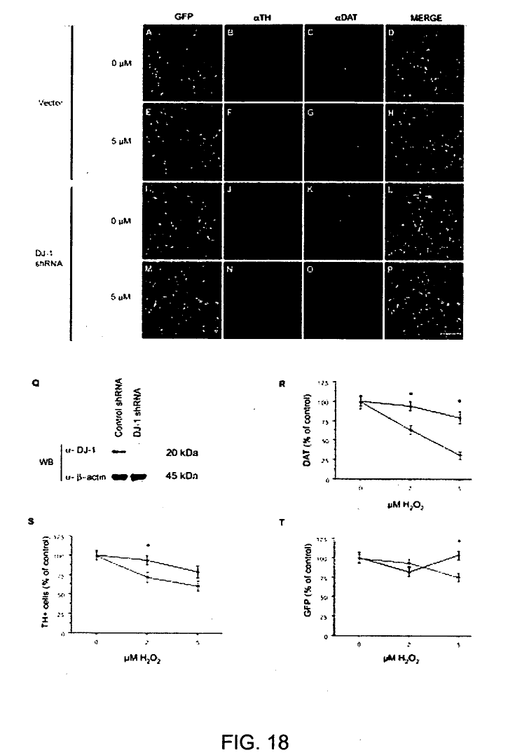

To confirm the role of DJ-1 in primary midbrain dopamine neurons, DJ-1 expression was inhibited by RNA interference (RNAi) in embryonic day 13 (E13) murine primary midbrain cultures by lentiviral transduction of short hairpin RNAs (shRNA) (Rubinson et al. 2003). E13 midbrain cultures (Staropoli et al. 2003) were transduced with a lentiviral vector that encodes a fluorescent marker gene, eGFP, along with short hairpin RNAs (shRNA) homologous to murine DJ-1. DJ1-shRNA virus-infected cells displayed efficient silencing of DJ-1 gene expression to 10-20% of control vector-infected cultures (as determined by Western blotting; FIG. 18Q). Transduction efficiency, as assessed by visualization of the fluorescent eGFP marker, exceeded 95% in all cases (FIG. 181 and data not shown). After 7 days in vitro (DIV7), cultures were exposed to hydrogen peroxide for 24 hours and then evaluated for dopamine neuron survival as quantified by immunostaining for TH and DAT.

Midbrain cultures transduced with DJ-1 shRNA virus and control vector transduced cells displayed similar numbers of TH-positive neurons in the absence of exposure to H₂O₂ (FIGS. 5A-D, I-L, R-S). In contrast, in the presence of H₂O₂, DJ-1-deficient cultures displayed significantly reduced dopamine neuron survival as quantified by immunohistochemistry for TH or DAT (FIGS. 18E-H, M-P, R-S). Similar results were obtained in three independent studies. The reduction in DAT immunoreactivity appears to be more robust than the reduction in TH cell number in the context of hydrogen peroxide; this may reflect the differential localization of DAT to dopamine neuron processes, whereas TH is primarily in the cell body.

As described in a previously by the inventors, non-dopaminergic cells in the E13 primary midbrain cultures are predominantly GABAergic neurons (90-95%) (Staropoli et al. 2003). Total embryonic midbrain neurons transduced with either DJ-1 shRNA or vector displayed comparable survival in the context of toxin exposure, suggesting that DJ-1 deficiency leads to a relatively specific alteration in dopamine neuron survival (FIG. 18T). These data are consistent with the analyses of ES-derived dopamine neurons above and indicate that DJ-1 is required for the normal survival of midbrain dopamine neurons in the context of toxin exposure.

Discussion

In this study evidence is presented that DJ-1 is an essential component of the oxidative stress response of dopamine neurons. DJ-1 deficient cells display an apparently normal initial burst of ROS in response to H₂O₂, but they are unable to cope with the consequent toxicity, culminating in apoptosis. Additionally, the inventors found that DJ-1 deficiency sensitizes cells to the proteasomal inhibitor lactacystin but not other toxic stimuli such as tunicamycin. Proteasomal inhibition induces the accumulation of short-lived and misfolded cytoplasmic proteins, leading to oxidative stress and apoptosis (Demasi and Davies 2003). Reactive oxygen species and proteasomal inhibition have previously been correlated with PD pathology (Dauer and Przedborski 2003), and it is therefore tempting to hypothesize that DJ-1 mutations lead to PD due to an increased sensitivity to such stressors.

The apparent cell-type specificity of DN impairment in patients with Parkinsonism-associated DJ-1 mutation is not predicted by the ubiquitous expression of DJ-1 (Nagakubo et al. 1997). In this study, the inventors discovered that DJ-1 protects both dopaminergic and nondopaminergic cells from oxidative insult. However, DJ-1 deficient dopamine neurons appear to be especially sensitive to oxidative insult, suggesting relative cell-type specificity to the consequences of DJ-1 deficiency. Similar results are observed with knockout ES-derived dopamine neurons that are devoid of any detectable DJ-1 and in primary dopamine neurons with DJ-1 levels reduced by RNAi ‘knockdown’. However, the inventors found that even in the absence of exogenous toxin exposure, ES-derived DJ-1‘knockout’ DNs display reduced survival, whereas the primary embryonic midbrain ‘RNAi knockdown’ DNs appear similar to wild-type cells. The inventors hypothesize that this discrepancy reflects the activity of residual DJ-1 (approximately 10-20%) in the primary midbrain ‘knockdown’ cultures. Alternatively, the ES-derived ‘knockout’ DNs may be exposed to a greater degree of oxidative stress in vitro than the midbrain derived DNs even in the absence of added toxin. The mechanism by which dopamine neurons are preferentially targeted for destruction in the absence of DJ-1 is unclear. It has been proposed that dopamine neurons are subject to high levels of endogenous oxidative stress that may relate to dopamine metabolism (Jenner and Olanow 1998).

DJ-1 is structurally modified in the context of cellular oxidative stress (Mitsumoto and Nakagawa 2001), suggesting a possible function. Two recent studies (Yokota et al. 2003; Taira et al. 2004) investigated the role of DJ-1 in the oxidative stress response of neuroblastoma tumor cells. Both studies use RNAi to perturb the expression of DJ-1 in neuroblastoma tumor cell lines, and suggest that DJ-1 deficiency sensitizes to oxidative stress, consistent with this data. Taira et al. further report that overexpression of DJ-1 in neuroblastoma cells leads to a reduction in ROS accumulation and hypothesize that DJ-1 may harbor antioxidant activity in vivo. In contrast, the inventors found that ES cells that are deficient in DJ-1 display a normal initial burst of ROS in the context of H₂O₂. Consistent with this, antioxidant activity in vitro was not detected (S.B. and A. A., data not shown).

Finally, this study presents a novel, ES cell-based genetic approach to the study of neurodegenerative disorders. Mouse genetic models of disease are often limited by the inherent variability of animal experiments, the limited mouse lifespan, and by difficulties in manipulating whole animals. For instance, genetic rescue experiments and toxicological dose-response studies are impractical in whole animals. Furthermore, genetic cell models are more readily amenable to molecular dissection of disease mechanism. Thus, genetically altered, ES-derived neurons are likely to be generally useful as cellular models of these disorders. Future studies may also utilize available human ES cells to investigate species differences.

Methods

Cell Culture. Undifferentiated ES cells were cultured using standard techniques (Abeliovich et al. 2000). SDIA differentiation of ES cultures was performed as described (Kawasaki et al. 2000) except that ES cells were plated at a density of 500 cells/cm² and cocultured with the MS5 mouse stromal cell line (Barberi et al. 2003). Transfections were performed using Lipofectamine 2000 (Life Technologies) for 18-36 hours as per the manufacturer's instructions (Staropoli et al. 2003). Primary cultures and infections were performed as described (Staropoli et al. 2003).

Antibodies. An anti-DJ-1 rabbit polyclonal antibody was generated against the synthetic polypeptide QNLSESPMVKEILKEQESR, which corresponds to amino acids 64-82 of the mouse protein. Antiserum was produced using the Polyquick antibody production service (Zymed). The antiserum was affinity purified on a peptide-coupled Sulfolink column (Pierce) per the manufacturer's instructions. Antibody was used at a dilution of 1:200 for immunohistochemistry and Western blotting as described (Staropoli et al. 2003). Immunohistochemistry was performed with a rabbit polyclonal antibody to TH (PelFreez; dilution 1:1000), a mouse monoclonal antibody to TujI (Covance, dilution 1:500), and a rabbit polyclonal antibody to GABA (Sigma, dilution 1:1000). Western blotting was performed using cleaved PARP polyclonal antibody (Cell Signaling, dilution 1:500), monoclonal DJ-1 antibody (Stressgen, dilution 1:1000) and β-Actin (Sigma, 1:500).

In Vivo Assays. ES cells plated in 96-well format (2.3×10⁴ cells/well) were treated for 15 hours with H₂O₂ in ES media deficient in β-mercaptoethanol (Abeliovich et al. 2000). Cell viability (as a percent of untreated control) was determined by MTT assay in triplicate (Fezoui et al. 2000). Annexin V/Propidium Iodide (Molecular probes) staining was performed per the manufacturer's instructions. For dihydrorhodamine-123 staining (DHR, Molecular probes) (Walrand et al. 2003), cells were preincubated for 30 min with DHR (5 μM), washed with PBS, then treated with H₂O₂ in ES media deficient in β-mercaptoethanol for 15 min at 37° C. The FACS analysis was performed using a FACSTAR sorter (Becton Dickinson). Dopamine uptake assay was performed essentially as described (Farrer et al. 1998). Reported values represent specific uptake from which non-specific uptake, determined in the presence of mazindol, was subtracted, and normalized for protein content (BCA kit, Pierce).

Primary midbrain embryonic cultures were prepared and transduced with lentiviral vectors as described (Staropoli et al. 2003). DJ-1 shRNA vector was generated by insertion of annealed oligonucleotides 5′-TGTCACTGTTGCAGGCTTGGTTCAAGAGACCAAGCCTGCAACAGTG ACTTTTTTC-3′ and 5′-ACAGTGACAACGTCCGAACCAAGTTCTCTGGTTCGGACGTTGTCACTG AAAAAAGAGCT-3′ into the LentiLox vector (Rubinson et al. 2003). For cellular dopamine quantification, cultures were incubated in standard differentiation media supplemented with L-DOPA (50 μM) for 1 hour to amplify dopamine production as described (Pothos et al. 1996). Subsequently cells were washed in phosphate buffered saline and then lysed in 0.2 M perchloric acid. Dopamine levels were quantified by HPLC (Yang et al. 1998) and normalized for protein content as above.

Expression Vectors. cDNA for DJ-1 was PCR amplified from human liver cDNA (Clontech) and cloned into the expression vectors pET-28a (Novagen) or pcDNA3.1 (Invitrogen) using standard techniques. Flag-DJ-1 and all described mutants were generated by PCR-mediated mutagenesis.

Protein Carbonyl Analysis. For protein carbonyl quantitation (Bian et al. 2003), cells were plated (1.4×10⁵ cells per well), grown for 24 hours, and then treated with 10 μM H₂O₂ as indicated. Cleared lysate (40 μl) from each time point was added to 2 M HCl (120 μl) with or without 10 mM DNPH and incubated for 1 h at 24° C. with shaking. Proteins were then TCA precipitated and resuspended in 200 μl 6M Guanidinium Chloride. Absorbance was measured at 360 nm, and DNP-conjugated samples were normalized for protein concentration with the underivitized control samples.

### Example 3

DJ-1 Lacks Apparent Protease and Antioxidant Activities In Vitro

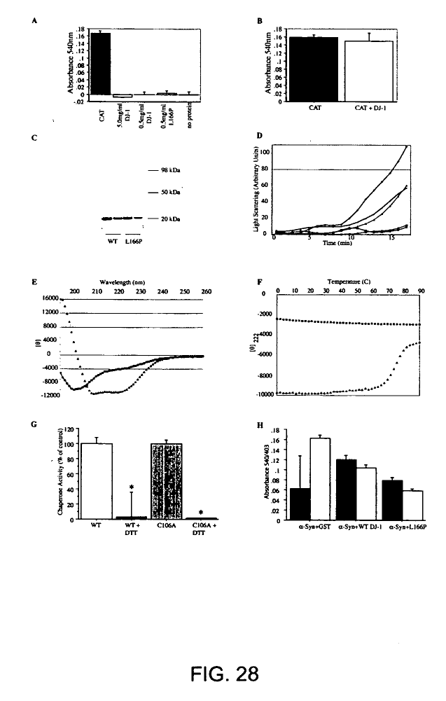

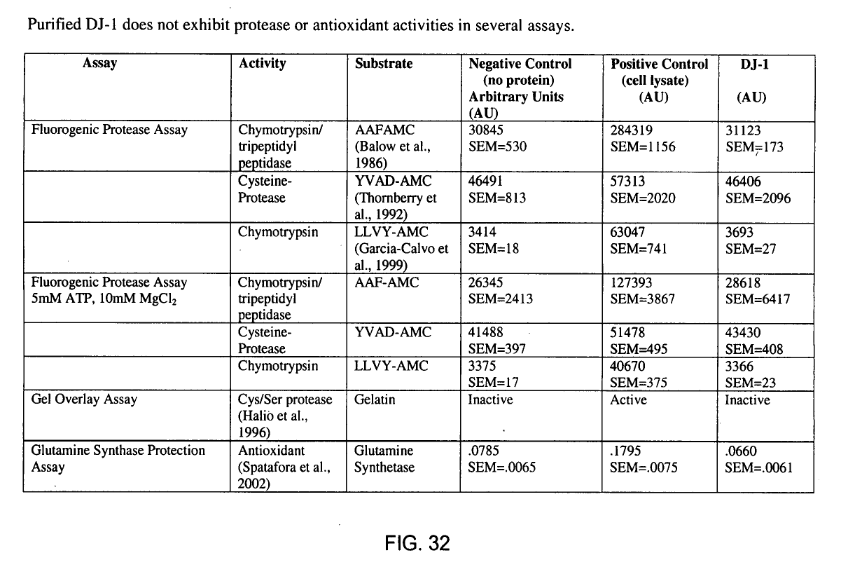

DJ-1 homologs have been reported to harbor protease (Halio et al. 1996; Du et al. 2000; Lee et al. 2003) and amidotransferase activities (Horvath and Grishin 2001). However, crystal structure analyses of DJ-1 suggest that this protein may not retain such catalytic activities (Honbou et al. 2003a; Huai et al. 2003; Lee et al. 2003; Tao and Tong 2003; Wilson et al. 2003). Consistent with this, purified DJ-1 preparations failed to display in vitro protease activity toward a variety of synthetic or natural substrates, and, similarly, DJ-1 lacked antioxidant (FIG. 32) or catalase activities (FIG. 28) in vitro. Furthermore, cells deficient in DJ-1 appear unaltered in the initial accumulation of ROS in the context of acute oxidative stress (Martinat et al. 2004).

DJ-1 Is a Redox-Dependent Molecular Chaperone

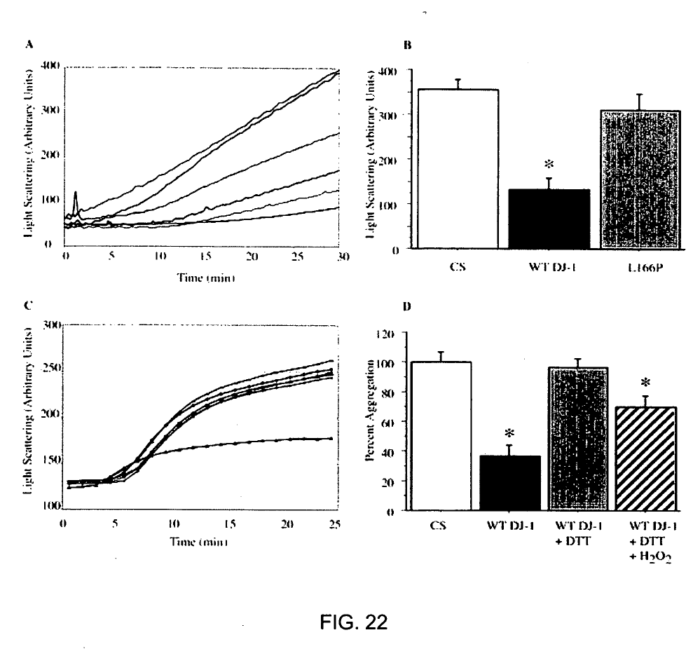

Every organism responds to ROS and other toxic environmental stresses by overexpressing a highly conserved set of heat shock proteins (Hsps), many of which function as molecular chaperones to assist other proteins in folding. Hsp31, an E. coli ThiJ domain protein, has been shown to function as a molecular chaperone in vitro (Sastry et al. 2002; Malki et al. 2003). The inventors hypothesized that DJ-1 may similarly function as a protein chaperone to protect cells from ROS. DJ-1 chaperone activity was quantified in the suppression of heat-induced aggregation of citrate synthase (CS) and glutathione S-transferase (GST), two well-characterized protein chaperone assays. These proteins lose their native conformation and undergo aggregation during incubation at 43° C. and 60° C., respectively. Addition of 0.5-4.0 μM polyhistidine (His)-tagged DJ-1 was found to effectively suppress the heat-induced aggregation of 0.8 μM CS (FIG. 22A). The chaperone activity was independent of the His tag used for purification, as cleavage and removal of the His tag did not alter DJ-1 chaperone function (unpublished data). DJ-1 chaperone activity is comparable to that of a well-described small cytoplasmic chaperone, human Hsp27. In contrast, RNase A failed to demonstrate chaperone activity and served as a negative control. Interestingly, the Parkinsonism-associated L166P DJ-1 mutation abrogated chaperone activity relative to the wild-type (WT) protein (FIG. 22B).

DJ-1 similarly functioned as a molecular chaperone in the context of the heat-induced aggregation of GST (FIG. 28). In contrast, DJ-1 failed to display activity in a third chaperone assay, aggregation suppression of reduced insulin (FIG. 22C). Reduction of the disulfide bonds between the A and B chains of insulin with dithiothreitol (DTT) leads to aggregation of the B chains. Hsp27 effectively inhibited the aggregation of insulin in the presence of 20 mM DTT, whereas neither DJ-1 nor the negative control protein RNase A displayed chaperone activity in this assay. As the insulin aggregation assay is performed in a reduced environment, the inventors hypothesized that DJ-1 chaperone activity may be redox regulated. Interestingly, such a redox switch in a molecular chaperone has been described in Hsp33 (Jakob et al. 1999), a dimeric bacterial Hsp unrelated to DJ-1.

To test the redox regulation of DJ-1, the inventors assayed chaperone activity in the CS aggregation assay in the presence or absence of the reducing agent DTT. DJ-1 chaperone activity in the CS aggregation assay was completely abrogated by preincubation of DJ-1 with 0.5 mM DTT in aggregation buffer for 10 min at 4° C. (FIG. 22D). DTT did not significantly alter CS aggregation in the absence of DJ-1 and did not modify suppression of CS aggregation by Hsp27 (unpublished data). To further test whether redox regulation might govern DJ-1 chaperone activity, reactivation studies using reduced DJ-1 were performed. DTT-reduced DJ-1 was incubated with H2O2 (10 mM in aggregation buffer for 10 min at 4° C. followed by dialysis against aggregation buffer for 2 h), and subsequently chaperone activity was measured in the CS thermal aggregation assay. H2O2 effectively reactivated the chaperone activity of DTT-treated DJ-1 (FIG. 22D). This was not an indirect effect of residual H2O2 on CS aggregation, as H2O2 treatment of CS increased aggregation (unpublished data). These results suggest that redox regulation of DJ-1 is reversible and is regulated by the redox environment.

Molecular chaperones typically display marked stability to thermal stress (Sastry et al. 2002). Consistent with this, the ultraviolet-circular dichroism (CD) spectrum of WT DJ-1 is consistent with a well-folded protein, and thermal denaturation of WT DJ-1 revealed a cooperative thermal unfolding transition at approximately 75° C. (FIG. 28). In contrast, the CD spectrum of the DJ-1 L166P mutant protein is typical of a partially unfolded polypeptide, suggesting that the L166P mutation causes a significant loss of helical structure. The mutant protein does not exhibit a thermal unfolding transition in the range studied (0-90° C.).

DJ-1 Inhibits the Generation of αSyn Aggregates

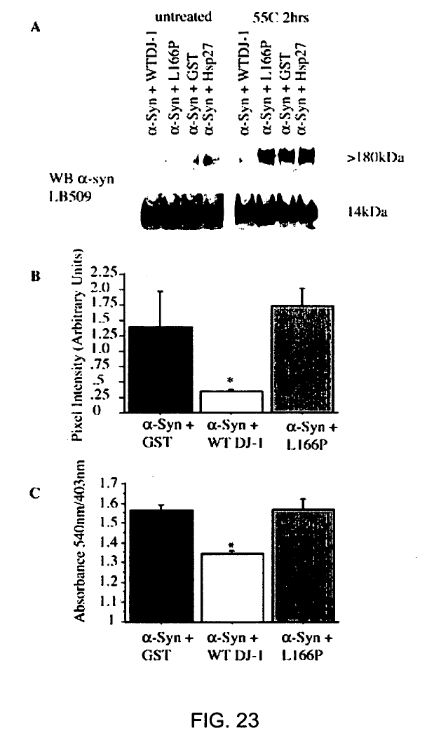

The analysis of DJ-1 chaperone function to a candidate DJ-1 substrate, αSyn (FIG. 23) was extended. The aggregation of αSyn has been implicated in familial and sporadic forms of PD, as mutations associated with autosomal dominant familial primary Parkinsonism alter the propensity of αSyn to aggregate (Conway et al. 2000a), and as αSyn fibrils are a major constituent of the Lewy body intracytoplasmic inclusions that typify PD pathology (Spillantini et al. 1997). In vitro, monomeric αSyn is disordered or “natively unfolded” in dilute solution (Weinreb et al. 1996). Incubation of purified WT human αSyn for 2 h at 55° C. results in the generation of high molecular weight multimers that likely represent protofibrils (FIGS. 23A and 23B) (Volles et al. 2001; Gosavi et al. 2002). This treatment does not result in formation of mature amyloid fibrils, as determined by Congo red staining (FIG. 28). WT DJ-1 effectively inhibits the formation of soluble αSyn protofibrils at a molar ratio of 1:2 (DJ-1: αSyn). In contrast, L166P mutant DJ-1, GST, and Hsp27 (FIGS. 23A and 23B) failed to inhibit the generation of αSyn protofibrils.

αSyn protofibrils have been shown to be an intermediate in the formation of mature amyloid fibrils. Because DJ-1 chaperone activity is effective at inhibiting the accumulation of αSyn protofibrils, the inventors sought to investigate the role of this activity in the generation of Congo red-positive mature fibrils. Congruently, WT DJ-1 inhibited formation of Congo red-positive αSyn fibrils, while L166P DJ-1 and GST did not (FIG. 23C). Thus, DJ-1 seems to inhibit formation of αSyn fibrils by preventing formation of αSyn high molecular weight oligomers, or protofibrils. Interestingly, PD-associated clinical mutations in αSyn appear to accelerate oligomerization and protofibril formation (Volles et al. 2001).

DJ-1 Chaperone Activity In Vivo

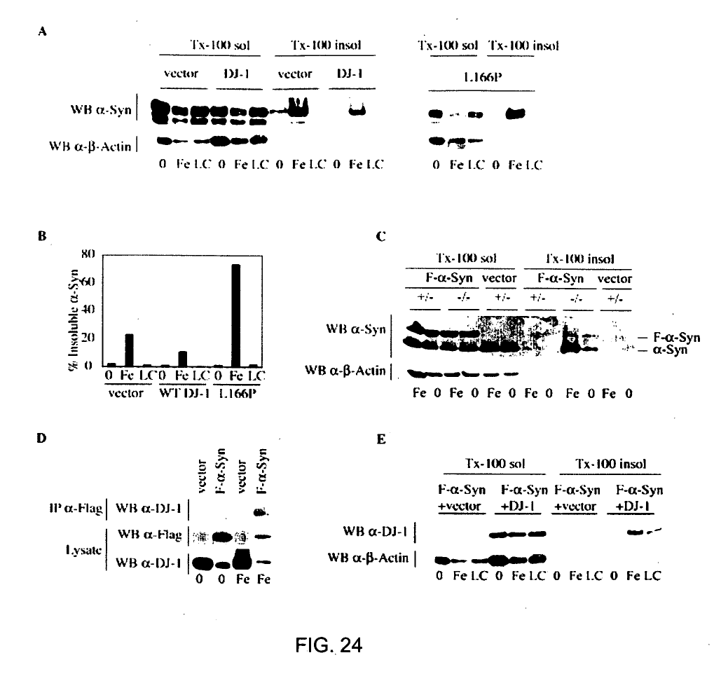

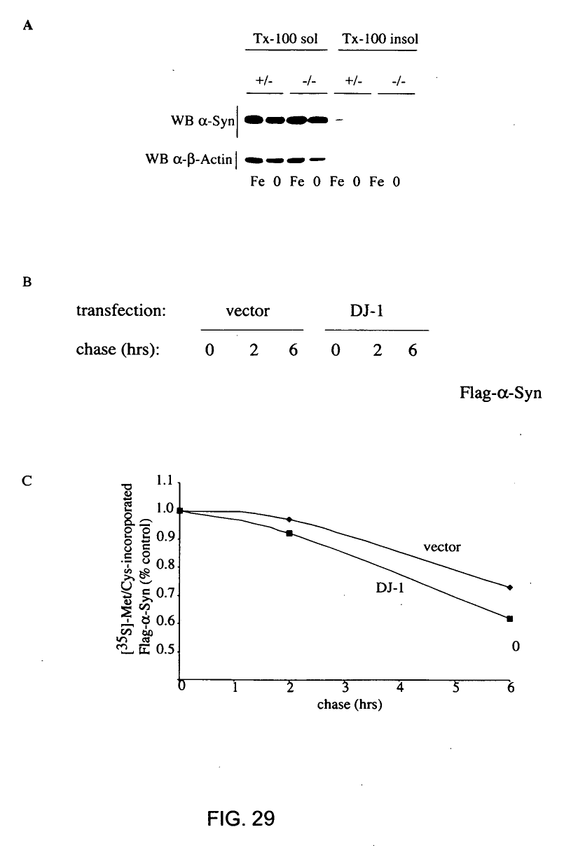

The inventors sought to investigate the chaperone activity of DJ-1 toward αSyn in vivo. αSyn has been shown to form aggregates that consist of both protofibrils and mature amyloid fibrils in the context of oxidative stress (such as FeCl2 treatment [Lee and Lee 2002; Lee et al. 2002]) in neuroblastoma cells. The activity of DJ-1 overexpression on αSyn aggregation in this tissue culture model system was evaluated. Briefly, CAD murine neuroblastoma cells (Staropoli et al. 2003) were transfected with Flag epitope-tagged αSyn (Flag-αSyn), differentiated via serum withdrawal, and exposed to FeCl2 (2 mM) for 18 h. Treatment with FeCl2 induced accumulation of αSyn in the Triton X-100-insoluble fraction, which has been shown to correlate with αSyn protofibrils (Lee and Lee 2002). Overexpression of WT DJ-1, but not L166P clinical mutant DJ-1, significantly inhibited the accumulation of Triton X-100-insoluble αSyn (FIGS. 24A and 24B). DJ-1 overexpression did not alter the accumulation (FIG. 24A) or half-life of soluble αSyn, as determined by pulse-chase kinetic analysis (FIG. 29). Thus, DJ-1 overexpression is sufficient to inhibit the formation of αSyn aggregates in vivo, consistent with the in vitro analysis.

To investigate whether DJ-1 is necessary to inhibit αSyn aggregation in vivo, the inventors utilized DJ-1 “knockout” embryonic stem (ES) cells, which display increased sensitivity to oxidative stress. DJ-1 homozygous knockout or control heterozygous ES cells (heterozygous cells were used as controls because they were the source of the knockout subclones) were differentiated in vitro using the embryoid body protocol (Martinat et al. 2004) and transfected with Flag-αSyn or control vector. Upon differentiation, both endogenous αSyn and transfected Flag-αSyn are accumulated to a similar extent in the soluble fraction of knockout and control cell lysates, as determined by Western blotting with an antibody for αSyn. In contrast, DJ-1-deficient cells (but not control cells) additionally accumulate Triton X-100-insoluble αSyn (both endogenous αSyn and transfected Flag-αSyn), which likely corresponds to protofibril aggregates (Lee and Lee 2002). As predicted, FeCl2 treatment further promoted the accumulation of insoluble αSyn in DJ-1-deficient cells but not in control heterozygous cells (FIG. 24C). Interestingly, transfection of Flag-αSyn into undifferentiated knockout or control ES cells in the presence or absence of FeCl2 treatment did not lead to the accumulation of insoluble Flag-αSyn (FIG. 29), consistent with a prior study suggesting a role for neuronal differentiation in the generation of insoluble αSyn aggregates (Lee et al. 2002).

To investigate the mechanism of DJ-1 activity toward αSyn, coimmunoprecipitation experiments on untreated and FeCl2-treated CAD cells transfected with DJ-1 and Flag-αSyn (or control vector) were performed as above. Triton X-100-soluble cell lysates were immunoprecipitated with a mouse monoclonal antibody for the Flag epitope, and Western blots were probed with a rabbit polyclonal antibody for DJ-1. DJ-1 failed to interact with Flag-αSyn in the absence of pretreatment with FeCl2, but an association was evident in FeCl2-treated cell lysates (FIG. 24D). Furthermore, overexpression of αSyn (but not vector control) leads to a reduction in the soluble pool of DJ-1, particularly in the context of FeCl2 treatment, indicating that DJ-1 additionally associates with an insoluble fraction of αSyn (FIG. 24D, bottom panel). Consistent with this, the inventors found that a significant fraction of DJ-1 protein localizes to the insoluble fraction upon FeCl2 treatment (FIG. 24E) in cells that have been cotransfected with Flag-αSyn.

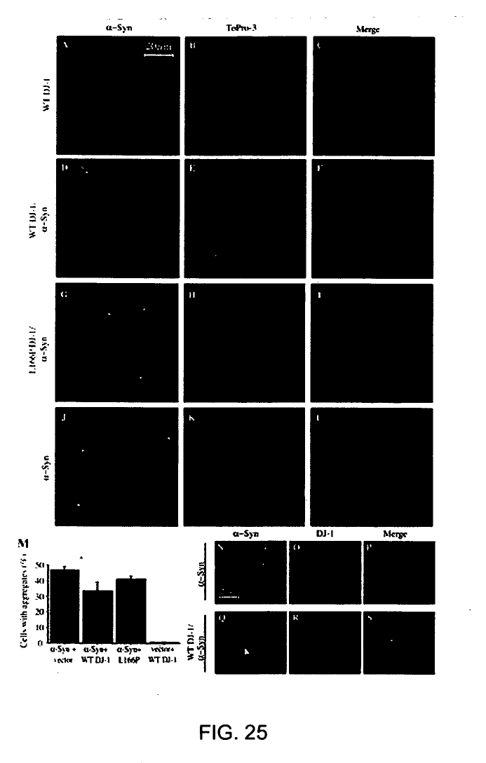

To further evaluate αSyn aggregation, the inventors performed immunohistochemical analyses of CAD cells transfected with αSyn along with DJ-1 or control vector (FIG. 25). Overexpression of αSyn in neuroblastoma cells induces the formation of visible cytoplasmic aggregates (Lee and Lee 2002) (FIGS. 25J-25L). Additional overexpression of WT DJ-1 significantly decreased the number of cells containing αSyn aggregates (FIGS. 25D-25F and 25M), whereas the L166P DJ-1 mutant fails to do so (FIGS. 25G-25I and 25M). However, DJ-1 does not appear to colocalize with αSyn aggregates, suggesting that DJ-1 functions at an early step in the formation of mature aggregates (FIGS. 25N-25S).

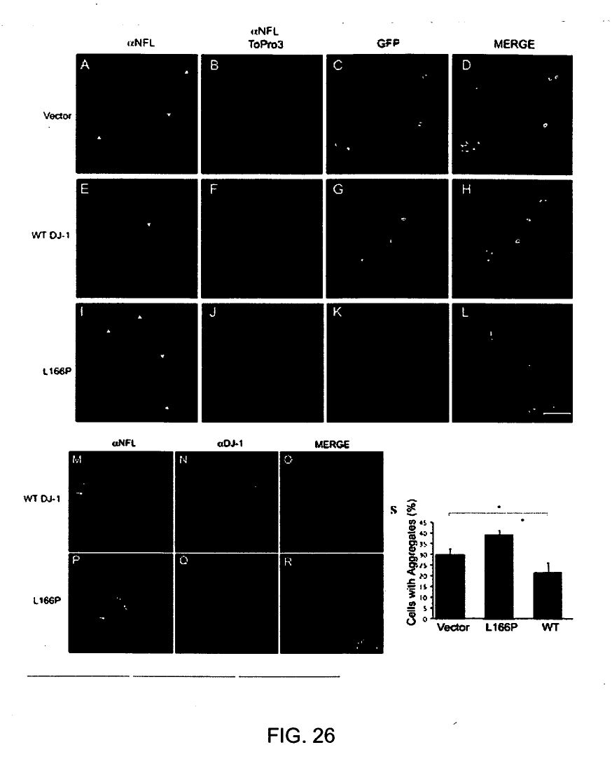

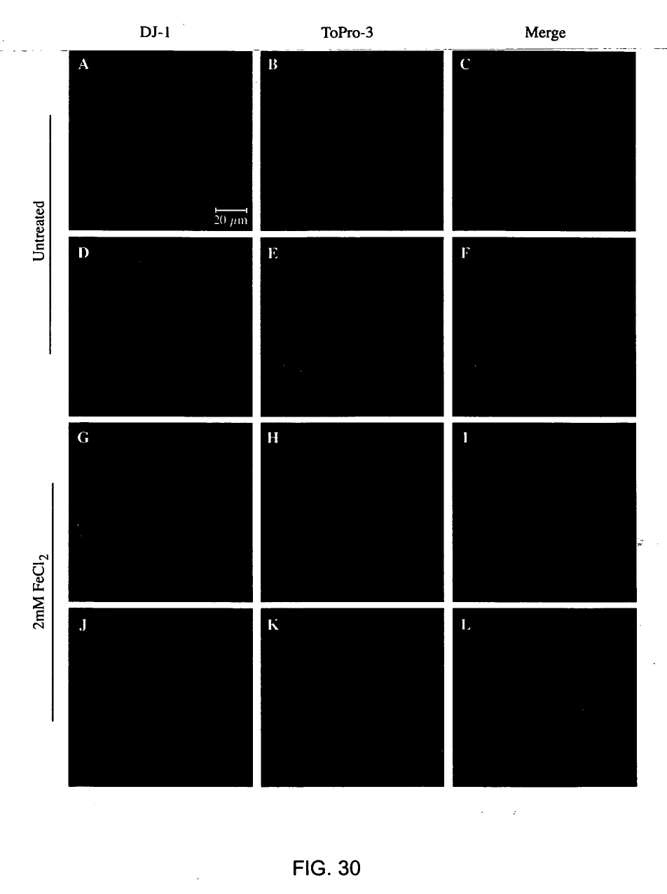

In a separate set of experiments, the inventors assayed the ability of DJ-1 to inhibit aggregation of a second substrate, neurofilament light subunit (NFL). Overexpression of a mutant form of human NFL, Q333P, by transient transfection of CAD murine neuroblastoma cells, leads to the accumulation of intracytoplasmic inclusions (Perez-Olle et al. 2002). Co-overexpression of WT DJ-1 along with mutant NFL significantly inhibited the accumulation of NFL inclusions (FIG. 26), whereas overexpression of the L166P Parkinsonism-associated mutant form of DJ-1 with NFL failed to inhibit the accumulation of aggregates. Coimmunostaining for DJ-1 and NFL indicated that DJ-1 does not colocalize with the NFL inclusions (FIGS. 26M-26R). DJ-1 did not appear to alter the expression of NFL (FIG. 30). These data are consistent with this analysis of DJ-1 chaperone activity toward αSyn and indicate that DJ-1 harbors chaperone activity toward a range of substrates in vivo.

DJ-1 Function Requires Cysteine 53

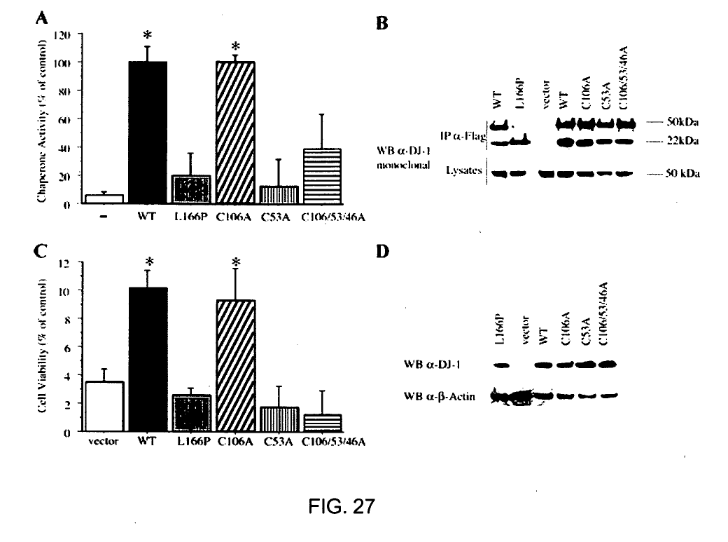

The DJ-1 crystal structure suggests the presence of two highly reactive cysteines, cysteine 106 (Lee et al. 2003; Wilson et al. 2003) and cysteine 53 (Honbou et al. 2003b). To test whether reactive cysteines play a critical role in the function or regulation of DJ-1 activity, the inventors mutagenized each cysteine in DJ-1 to alanine (FIG. 27). Surprisingly, mutation of cysteine 106, at the predicted nucleophile elbow of DJ-1, does not alter the basal activity (FIG. 27A) or the DTT sensitivity (FIG. 28) of DJ-1 chaperone function. In contrast, mutation of cysteine 53, which is present at the dimeric interface of DJ-1, completely abrogates chaperone activity. Similarly, mutation of all three cysteines in DJ-1 (cysteine 106, cysteine 53, and cysteine 47) leads to the loss of chaperone function. The cysteine mutations do not alter DJ-1 dimerization (FIG. 27D) or the apparent stability of DJ-1 in vivo (unpublished data), unlike the L166P Parkinsonism-associated mutation.

DJ-1-deficient ES cells display increased sensitivity to oxidative stress, and this phenotype can be “rescued” by overexpression of WT DJ-1 but not PD-associated L166P mutant DJ-1 (Martinat et al. 2004). The inventors further investigated the activity of the cysteine-mutant forms of human DJ-1 in vivo in the complementation of DJ-1-deficient ES cells. Cysteine 106-mutant DJ-1 robustly rescued DJ-1 knockout cells from H2O2 toxicity, consistent with the in vitro chaperone activity assay (FIG. 27C). In contrast, cysteine 53 and the triple-cysteine mutant forms of DJ-1 failed to protect from H2O2 toxicity. These data support a role for cysteine 53-dependent chaperone activity in DJ-1-mediated ROS protection, and demonstrate a direct correlation between DJ-1 in vitro chaperone activity and cellular protection from oxidative stress. These data are consistent with the prior observation that mutation of cysteine 53 to alanine abrogates the low-isoelectric point variant that is induced by oxidative stress (Honbou et al. 2003a).

The inventors provide evidence that DJ-1 functions as a cytoplasmic redox-sensitive molecular chaperone in vitro and in vivo. This activity extends to αSyn and the neurofilament subunit NFL, proteins implicated in PD pathology. In a companion article (Martinat et al. 2004), the inventors show that DJ-1 deficiency sensitizes cells to oxidative stress, leading to increased apoptosis in the context of an ROS burst. Taken together, these data strongly support the notion that DJ-1 functions as a redox-dependent protein chaperone to mitigate molecular insults downstream of an ROS burst. Oxidation-modified proteins have been shown to accumulate in the context of normal aging and PD, and may participate in the generation of protein aggregates in neurodegenerative disorders (Jenner 2003).

It is of interest to identify relevant in vivo substrates for DJ-1 activity in the context of DNs in PD. These data suggest that DJ-1 activity extends to multiple targets, reminiscent of other small protein chaperones (Gusev et al. 2002), and consistent with this, DJ-1 activity is not ATP-dependent (unpublished data). Candidate substrates for DJ-1 chaperone activity in the context of PD include αSyn and neurofilament proteins, based on their presence in PD protein inclusions. These data suggest that DJ-1 functions to suppress protein aggregates in the cytoplasm. It is possible that DJ-1 plays additional roles in the mitochondria or nucleus, as has been suggested (Bonifati 2003; Canet-Aviles 2004), although DJ-1 appears to remain localized diffusely in the cytoplasm with or without toxin treatment in these studies (FIG. 29).

These data indicate that DJ-1 can suppress an early step in the formation of αSyn aggregates, the generation of high molecular weight oligomers (protofibrils). Interestingly, it has been suggested that such protofibrils, rather than the large fibrillar aggregates, may underlie αSyn toxicity in vivo (Volles et al. 2001). DJ-1 inhibits the aggregation of αSyn in differentiated cells in vivo, and loss of DJ-1 leads to increased accumulation of insoluble αSyn. DJ-1 appears to associate with αSyn in the Triton X-100-soluble fraction of FeCl2-treated lysates, and DJ-1 colocalizes with αSyn in the Triton X-100-insoluble fraction in the context of FeCl2 treatment. However, DJ-1 does not colocalize with the punctate protein aggregates visible by immunostaining in the case of either αSyn or NFL. This supports the notion that DJ-1 functions at an early step in the aggregation process, when the substrate protein may be misfolded, but has not yet formed a mature aggregate. The inventors hypothesize that DJ-1 may promote the degradation of such misfolded proteins, either through the proteasome or through other cellular pathways such as chaperone-mediated autophagy.

A recent study investigated the chaperone activity of WT DJ-1 in vitro toward CS and concluded that redox regulation was not a significant factor (Lee et al. 2003). This is most likely a consequence of the use of only oxidizing conditions (0.5 mM H2O2) but not reducing conditions in the described chaperone assays (Lee et al. 2003). A second report failed to detect DJ-1 chaperone activity in vitro (Olzmann et al. 2003), but importantly, this study employed only reducing conditions in which DJ-1 chaperone activity is abrogated. In the present study the inventors demonstrate that DJ-1 chaperone activity is inhibited by reducing conditions, and can be stimulated by oxidation. Thus, in the normal reducing environment of the cell, DJ-1 may be inactive. Production of ROS and alteration of the redox state of the cytoplasm may activate DJ-1 chaperone activity as a mechanism of coping with protein aggregation and misfolding.

The inventors found that PD-associated L166P mutant DJ-1 fails to function as a molecular chaperone in vivo or in vitro. Consistent with this, in a companion article (Martinat et al. 2004), the inventors show that this mutant fails to complement DJ-1 knockout cells in vivo, even when overexpressed at artificially high levels (Martinat et al. 2004). Furthermore, the L166P mutant form fails to dimerize even when expressed at WT levels. Thus, although prior studies (Miller et al. 2003) and the inventors' analyses (unpublished data) have found that the L166P PD-associated DJ-1 mutation leads to decreased protein stability, it is apparent that even overexpression of the L166P mutant protein does not restore function. The L166P clinical phenotype is not due simply to reduced levels of DJ-1 protein, and, furthermore, evidence of altered subcellular localization of the L166P mutant protein was not observed (FIGS. 25M-25R). Rather, these studies favor a model by which the pathological mechanism of this mutation is a consequence of altered structure and resultant loss of function.

Mutation of cysteine 53 in DJ-1 abrogates both chaperone and protective functions of this protein. Interestingly, cysteine 53 has previously been implicated as a reactive cysteine required for the in vivo modification of DJ-1 to a lower isoelectric point in response to oxidative stress (Honbou et al. 2003a), consistent with a role for such redox regulation in vivo. In contrast, cysteine 106, which has been reported to be sensitive to oxidative modification in vitro (Wilson et al. 2003), does not appear to be required for the in vitro and in vivo DJ-1 activities.

Cell culture and in vivo assays. Undifferentiated ES cells, CAD neuroblastoma cells, and HeLa cells were cultured using standard techniques (Abeliovich et al. 2000; Staropoli et al. 2003). Transfections were performed using Lipofectamine 2000 (Life Technologies, Carlsbad, Calif., United States) for 18-36 h according to the manufacturer's instructions.

For in vivo αSyn aggregation assays, CAD cells were transfected with Flag-αSyn (pcDNA3) or DJ-1 (pCMS), and medium was replaced with medium without serum. Cells were cultured without serum to induce differentiation for 48 h post-transfection, at which time the medium was exchanged for medium alone or containing 2 mM FeCl2 and 5 μM lactacystin. Cells were treated with toxin for 18 h, then lysed or fixed with 4% PFA. Cell lysis was performed by resuspending cells in 50 mM Tris (pH 7.6), 150 mM sodium chloride, 0.2% Triton X-100, and protease inhibitor cocktail (Sigma, St. Louis, Mo., United States). Cells were incubated on ice for 20 min and Triton X-100-soluble and -insoluble fractions were separated via centrifugation at 13,000 rpm for 15 min.

Quantification of CAD cell aggregates was performed using a Zeiss LSM Pascal confocal microscope (Zeiss, Oberkochen, Germany) with a 20× long working distance lens. Images were imported to NIH Image J for analysis. Images from tenrandomly selected fields in each of three wells were quantified for each condition. Cells containing at least one intracytoplasmic aggregate, independent of size or number per cell, were scored as positive for aggregates. This number was divided by the number of transfected cells per field, determined by GFP fluorescence.

ES cell culture and in vitro differentiation. Mouse ES cells were propagated and differentiated as described (Martinat et al. 2004). ES cells were differentiated via the embryoid body protocol. Cells were transfected with Flag-αSyn (pCMS) using Lipofectamine 2000 as per the manufacturer's instructions. 48 h post-transfection, cells were treated with 2 mM FeCl2 (or media alone) for 18 h.

Antibodies. An anti-DJ-1 rabbit polyclonal antibody was generated against the synthetic polypeptide QNLSESPMVKEILKEQESR, which corresponds to amino acids 64-82 of the mouse protein. Antiserum was produced using the Polyquick polyclonal antibody production service of Zymed Laboratories (South San Francisco, Calif., United States). The antiserum was affinity purified on a peptide-coupled Sulfolink column (Pierce Biotechnology, Rockford, Ill., United States) according to the manufacturer's instructions. Antibody was used at a dilution of 1:200 for immunohistochemistry and Western blotting as described (Staropoli et al. 2003). Immunohistochemistry was performed with a rabbit polyclonal antibody to DJ-1 (Martinat et al. 2004), TH (PelFreez, Rogers, Ariz., United States; dilution 1:1000), and a rabbit polyclonal antibody to GABA (Sigma; dilution 1:1000). Western blotting was performed using monoclonal antibody to DJ-1 (Stressgen Biotechnologies, San Diego, Calif., United States; dilution 1:1000), a monoclonal antibody to αSyn LB509 antibody (Zymed), and a monoclonal antibody to β-actin (Sigma; dilution 1:500). Mouse monoclonal antibody to NFL (Sigma; dilution 1:200) and rabbit polyclonal antibody to NFL (Perez-Olle et al. 2002). ToPro3 (Molecular Probes, Eugene, Oreg., United States; dilution 1:1000) was used as a nuclear dye.

Expression Vectors

DJ-1 cDNA was PCR amplified from human liver cDNA (Clontech, Palo Alto, Calif., United States) and cloned into the expression vectors pET-28a (Novagen, Madison, Wis., United States) or pcDNA3.1 (Invitrogen, Carlsbad, Calif., United States). Flag-DJ-1 and all described mutants were generated by PCR-mediated mutagenesis using standard techniques.

In Vitro Preparation of WT and Mutant DJ-1

His-tagged recombinant human WT or L166P DJ-1 was produced in E. coli BL21 cells induced with 1 mM IPTG for 4 h at 37° C. Bacterial pellets were resuspended in 50 mM sodium phosphate (pH 6.8) and 300 mM sodium chloride, and lysed by sonication. Lysates were cleared by centrifugation at 20,000×g for 20 min, and the supernatant was incubated with NTA-Ni-conjugated agarose resin for 1 h at 4° C. The resin was subsequently washed five times with 20 resin volumes of lysis buffer containing 20 mM imidazole, and protein was eluted in five fractions of two resin volumes of lysis buffer containing 250 mM imidazole. Recombinant protein elutions were confirmed to be of >99% purity by SDS-PAGE and colloidal Coomassie staining.

Aggregation Assays

CS aggregation was performed in 40 mM HEPES (pH 7.8), 20 mM potassium hydroxinde, 50 mM potassium chloride, and 10 mM ammonium sulfate, and monitored in a thermostat-controlled fluorescence spectrophotometer with excitation and emission wavelengths at 500 nm and slit widths at 2.5 nm. Insulin aggregation was performed as described (Giasson et al. 2000). CS, insulin, RNase A, and GST were obtained from Sigma; human Hsp27 was obtained from Stressgen.

αSyn protofibril and fibril formation assays were performed essentially as described (Uversky et al.). Briefly, protofibrils were formed by incubation of 200 μM WT synuclein with 100 μM DJ-1 or control chaperone protein in PBS for 2 h at 55° C. Samples were mixed with SDS loading buffer and analyzed by SDS-PAGE and Western blotting using αSyn LB509 antibody (Zymed). Quantitation of high molecular weight αSyn was performed using NIH Image J. Integrated pixel intensity of high molecular weight synuclein for each sample was normalized to monomeric synuclein intensity. For fibril formation, αSyn and chaperone proteins (as described above) were incubated with shaking for 1 wk at 37° C. Fibril formation was assessed by Congo red (Conway et al. 2000b).

### Example 4

Generation of a ‘Marked’ Reporter ES Cell Line

To examine the process by which mouse ES cells acquire a dopaminergic phenotype, the inventors generated murine ES cell lines capable of giving rise to ‘marked’ mature DNs identifiable by the expression of enhanced yellow fluorescent protein (eYFP) or β-galactosidase (LacZ). The inventors employed a Cre-recombinase based 2-transgene approach, as has been broadly used in whole animals for cell type-specific and tissue-specific expression (Srinivas et al., 2001). Briefly, the phage-derived Cre recombinase is expressed specifically in the cells of interest—in this case midbrain dopamine neurons—along with a second transgene that harbors a marker gene under the regulation of Cre recombinase. To this end, a strain of mice was derived in which Cre recombinase was “knocked-in” at the dopamine transporter (DAT) locus, a ‘late’ marker of dopamine neurons (Zhuang et al., 2001). This marker is more specific than TH, as TH is also expressed in other catecholaminergic cell types such as norepinephrine cells in the locus ceruleus. Furthermore, DAT is expressed at a later developmental point than TH in vivo and in vitro (Barberi et al., 2003).

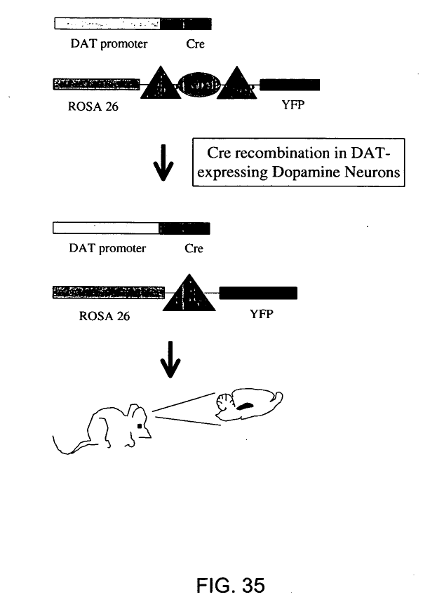

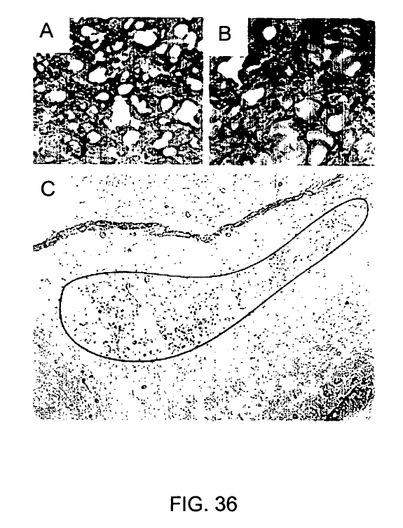

A second transgenic mouse line was obtained that harbors the eYFP (or LacZ) gene inserted into the constitutively-expressed ROSA26 locus, preceded by loxP-flanked stop sequence (Srinivas et al., 2001); thus, in cells expressing Cre recombinase, Cre-mediated excision of the loxP-flanked transcriptional stop sequence allows for marker gene expression. As predicted, the double transgenic progeny display expression of marker gene specifically in midbrain DNs (FIGS. 35 and 36)(Staropoli et al., 2003). Of note, these mice do not display any significant developmental defects (data not shown). ES cell lines were derived from double transgenic blastocysts using standard embryological technique (Wichterle et al., 2002). One double-transgenic ES cell clone, DY1, was demonstrated to be totipotent by injection into blastocysts and the generation of 100% ES-derived chimeric animals with germline transmission (FIG. 36).

Differentiation of DY1 ES Cells into ‘Marked’ Dopamine Neurons

Two established and complementary protocols to differentiate ES cells into DNs have been described. The embryoid body (EB) method (Lee et al., 2000) involves several steps: first, spherical cell aggregates (termed embryoid bodies) are generated that contain ectodermal, mesodermal and endodermal derivatives; second, these aggregates are selected for neuronal precursors and expanded with basic-FGF (bFGF); and third, differentiation is induced by growth factor withdrawal. DN differentiation is observed in vitro in terms of TH expression, an early marker of the dopamine lineage (Chung et al., 2002; Lee et al., 2000). There is also vesicular dopamine release, although this may be at a level that is significantly reduced below that found in primary midbrain cultures (Kim et al., 2002; Kim et al., 2003) (and consistent with the unpublished data).

A second protocol, called Stromal Cell-Derived Inducing Activity (SDIA), is a single step co-culture of ES cells and bone-marrow stromal cells (Kawasaki et al., 2002a). The molecular determinants of SDIA have not been defined but may represent multiple factors necessary for early neural induction as well as dopamine neuron specification. There is evidence for bone morphogenic signal (BMP) inhibition, which is known in vivo to be essential for the early specification of neural progenitors. This method appears to generate a higher percentage of TH-positive cells than the EB method (Barberi et al., 2003) and these cells appear capable of dopamine release in vitro, although (as with EB differentiation) dopamine levels may be at a significantly reduced level compared to primary midbrain cultures (although not compared directly (Bagri et al., 2002). Thus, these protocols may be inefficient at generating fully mature neurons in vitro. When transplanted into the striata of unilaterally 6-OHDA lesioned rodents, both EB and SDIA ES-derived DNs appeared to ‘rescue’ the amphetamine or apomorphine-induced turning behavior (Barberi et al., 2003; Kim et al., 2002). These data suggest the possibility that environmental determinants present in the adult CNS may be capable of inducing the terminal differentiation of transplanted dopamine neurons.

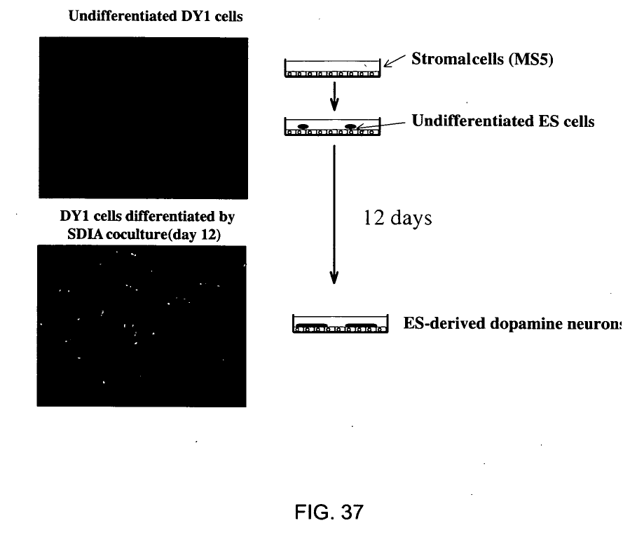

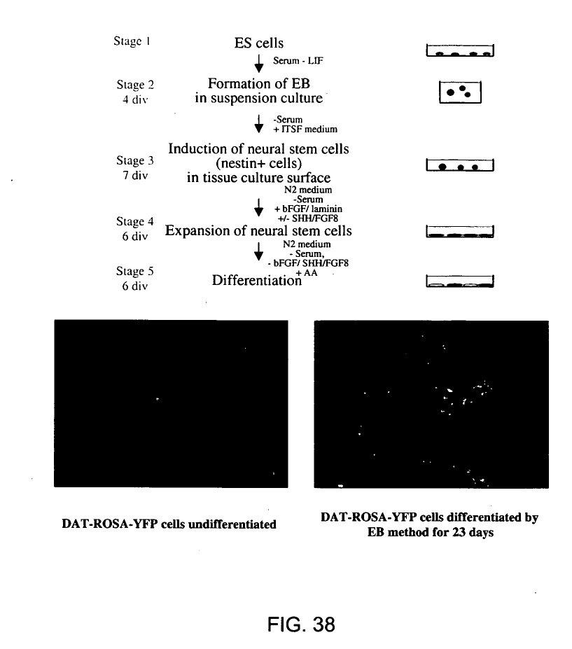

The differentiation of the DY1 ES cells, using either of these two protocols, gives rise to eYFP-positive cells (FIGS. 37 and 38). In contrast, few eYFP positive cells can be detected in non-differentiated cultures. The eYFP positive cells are specifically immunostained with a monoclonal antibody for DAT (FIGS. 37 and 38) and TH, confirming the restricted expression of eYFP to ‘late’ differentiated DNs. Appropriate fluorescence-conjugated secondary antibodies were used in immunostaining as described (Staropoli et al., 2003). As predicted, not all TH-immunostained cells are positive for eYFP. These results indicate that the DY1 ES cells can be differentiated in vitro into DNs and that these cells can be used to examine the specification of ES cells into DNs.

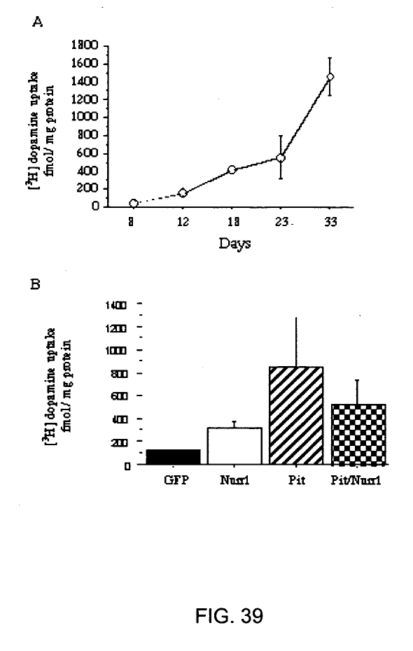

The inventors have further confirmed the differentiated state of the ES-derived dopamine neurons by quantifying additional dopamine neuron-specific markers and activities. The inventors have directly measured dopamine transporter activity in terms of the uptake of radioactive dopamine (Johnson et al., 1998). The inventors find that dopamine uptake activity is present in DY1 cultures differentiated by either SDIA or EB method, but this activity appears significantly later than TH and other markers. For instance, cells differentiated by SDIA display a low level of dopamine uptake activity at day 8, but this increased dramatically until day 30 (FIG. 39). In contrast, the inventors detect Nurr1 expression by RT-PCR at day 5, and TH expression as well as PitX3 expression are apparent at days 8 to 18 by quantitative real-time RT-PCR for mRNA (data not shown). As predicted, dopamine uptake activity in DY1 cells, which are hemizygous at the DAT gene locus, is reduced (relative to D3 wild-type cells; data not shown).

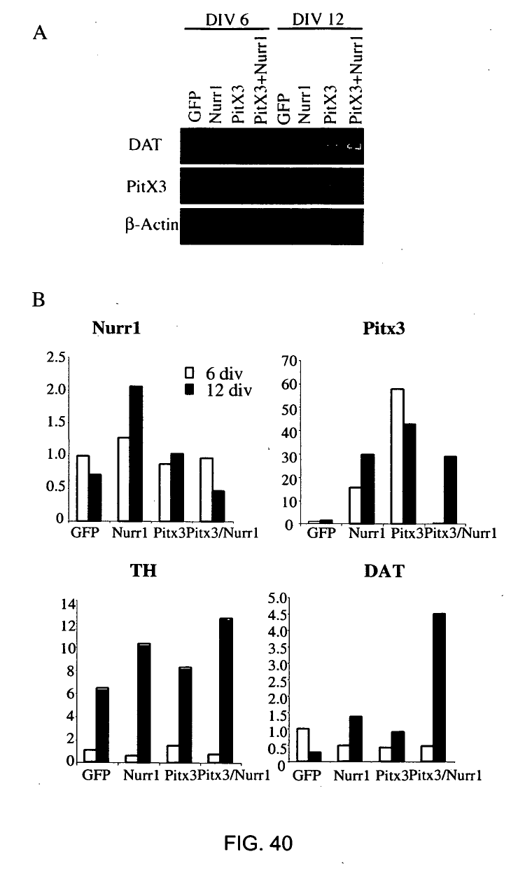

Similarly, the inventors have measured additional markers of the ‘late’ DN phenotype. Depolarization-induced dopamine release as well as total cellular dopamine (as a ratio of total cellular protein) is apparent at later stages of differentiation (data not shown). For instance, using the SDIA method, dopamine release is apparent at approximately days 12-14 (Barberi et al., 2003) and increase thereafter (data not shown), in parallel with DAT activity. The inventors have also measured a number of early and late markers of DN differentiation using real-time RT-PCR (FIG. 40). Total RNA was isolated using a standard protocol (Qiagen) from cultures at different time points of SDIA-mediated differentiation, and cDNA was generated (Invitrogen First Strand Kit).

Real-time PCR was performed as per the manufacturer's instructions (Cepheid) using oligonucleotides specific for genes that are expressed during the differentiation course. Relative mRNA concentrations were normalized to levels of β-actin as an internal control (Heid et al., 1996). TH first appears at day 8, and PitX3 appears at day 12 and thereafter. DAT expression at the RNA level is observed initially at day 12, consistent with immunohistochemistry and activity assays as described above (FIG. 40). Thus, the inventors have described multiple early and late markers of dopamine neuron differentiation, and these markers allow for a kinetic analysis of events in DN differentiation in vitro.

Lentiviral Vectors

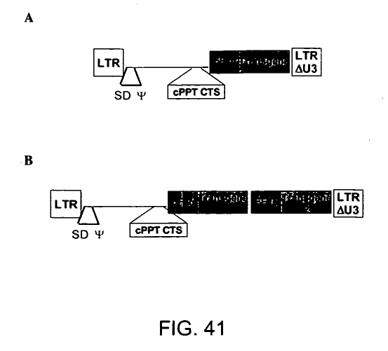

A network of TFs has been proposed to regulate the developmental program of specific neuronal populations, such as motor and dopamine neurons. Several TFs appear to be rather selectively expressed in post-mitotic midbrain DNs during development, including Nurr1, PitX3, and Lmx1b. Genetic studies have indicated a role for these factors in dopamine neuron development, but their mechanisms and points of action in the generation of ES cell-derived DNs have not been dissected. Furthermore, potential interactions between these factors have not been explored. The inventors have generated lentiviral vectors that express human Nurr1 or PitX3 cDNAs and transduce nearly 100% of cells in an ES culture and allow the overexpression of genes in mitotic or postmitotic cells (Zennou et al., 2001). Expression is induced over 20-fold, as confirmed by real-time quantitative RT-PCR (FIG. 8). Additionally, the inventors have generated vectors that harbor pairs of cDNAs, including PitX3 and Nurr1 together, or either PitX3 or Nurr1 along with a fluorescence marker such as dsRed2 (FIG. 41).

The inventors have also generated Lentilox (Rubinson et al., 2003) RNAi-based vectors that target the expression of Nurr1 and PitX3. Lentilox vectors harbor the U6 promoter to drive the expression of stem-loop sequences that mediated RNAi-based inhibition of target gene expression in order to knock-down the expression of sequences of interest, such as Nurr1 or PitX3 in postmitotic cells. The inventors have successfully knocked down gene expression by 90% in post-mitotic neurons using this technique (as ascertained by protein blotting; data not shown). All lentiviral vectors are prepared identically and quantified by p24 protein enzyme-linked immunoassay (Zennou et al., 2001).

Finally, the inventors have generated additional viral constructs that are Cre-inducible in that they harbor the identical lox-stop-lox cassette present in the marker transgene as present in the Rosa26 locus of DY-1 cells (data not shown). This allows for the conditional expression of virally transduced genes only in cells of interest that express the Cre recombinase—mature dopamine neurons. Initial analysis of this system using a dsRed marker transgene confirms its efficacy (data not shown). The inventors will generate additional constructs that allow for the specific expression of Nurr1 and PitX3 only in late-stage dopamine neurons.

PitX3 and Nurr1 Overexpression in ES-Derived DN Generation

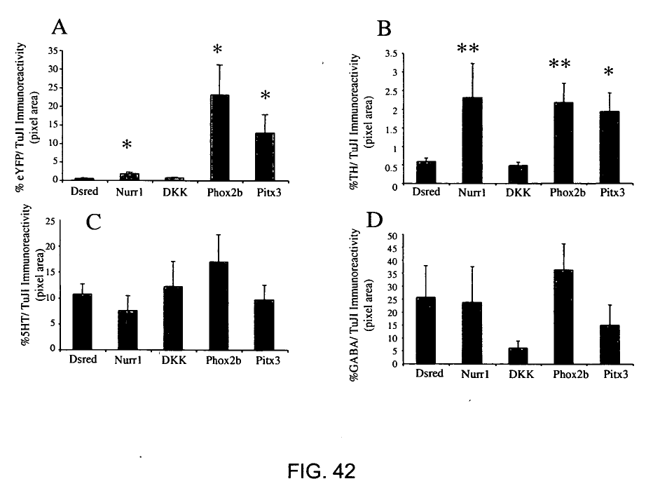

Using the ES cell differentiation assays and lentiviral vectors described above, the inventors have investigated whether these factors are necessary or sufficient to potentiate DN development from ES cells. DY1 cells were infected with control (dsRED), Nurr1, or PitX3 vectors 2 days prior to the initiation of differentiation. DN generation was quantified after either SDIA or EB differentiation by eYFP fluorescence or immunohistochemical analyses. The inventors confirmed the activating role of Nurr1 in the context of either differentiation protocol as quantified by eYFP expression of DY-1 cells (FIG. 42). Additionally, the inventors observed that PitX3 appears to be effective in this assay. Similar results were obtained by quantitative analysis of TH immunostaining (FIG. 42), whereas the generation of serotonin (5HT) and GABAergic neurons was not significantly altered. Also, dopamine uptake activity appears increased in cells that overexpress Nurr1 or PitX3 (FIG. 39). Real-time rt-PCR analysis shows that Nurr1 and PitX3 overexpression appear to synergize in inducing DAT (FIG. 40), and Nurr1 is effective in inducing PitX3 activity, consistent with an interaction between these pathways.

Additional Transcription Factors

The inventors also investigated the function of two other transcription factors in this pathway using viral overexpression. Phox2b is a transcription factor that plays a necessary and sufficient role in the induction of locus cerulius norepinephrine (NE) neurons, which arise in the dorsal hindbrain and express several markers in common with DNs. For instance, TH is a key enzyme in both cell types, and consistent with this, TH expression is induced efficiently by Phox2b overexpression. Surprisingly, the inventors find that Phox2b also efficiently induces the expression of eYFP in DY1 cells.

As Phox2b activity is normally restricted to dorsal precursors, one would not predict this. One explanation lies in the apparent dual roles of the Phox2 genes as early proneural genes (Dubreuil et al., 2002) as well as later patterning functions (Brunet and Pattyn, 2002). Thus, it is possible that Phox2b is functioning at an early point in the differentiation of DNs, which the inventors can test using additional phenotypic markers. It is interesting that two related Paired-like homeodomain genes, Phox2b and PitX3, function in this assay. Additional studies will also investigate the induction of NE markers in these cells by these factors.

Role of Brain Region-Specific Environmental Factors in Coculture

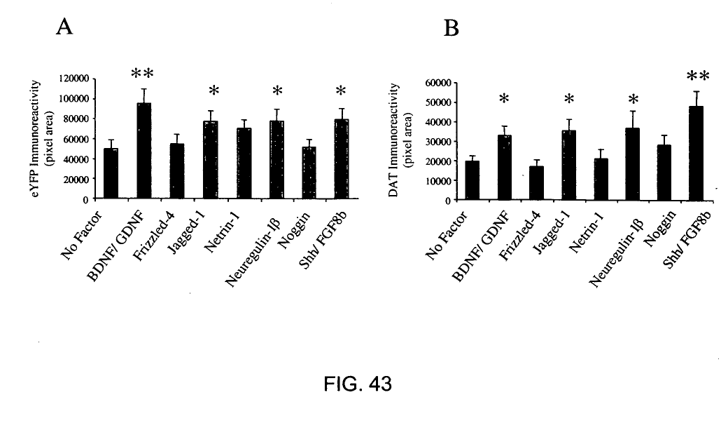

There is evidence that neuronal target tissues and support glial cells can influence the differentiation of dopamine neurons. The inventors sought to investigate whether such factors would modify the differentiation of ES-derived DNs. DY1 cells were differentiated using SDIA and subsequently ES cells were isolated using mild enzymatic (dispase) treatment (Kawasaki et al., 2002b) and replated on primary embryonic brain cultures obtained from E17 striatum (Prochiantz et al., 1979), cortex (at day 7 in vitro), or as a control onto another stromal cell layer. The preliminary results indicate that the generation of DAT-positive cells is increased in the context of primary cultures of striatum or cortex (FIG. 43). These data suggest that striatal or cortical cells stimulate the development of the dopaminergic neurons. However, as these are mixed primary cell cultures it remains unclear which cell type is responsible for this effect. Also, it is possible that the effect of these support cells might alter either the late differentiation of the DNs or their survival. Finally, the role of support tissue may be mediated through soluble factors or through cell-associated factors, particularly if the role of the support cells relates to synapse maturation.

Candidate Soluble Factors

The availability of marked ES-derived ‘late’ dopamine neurons should facilitate the analysis of candidate soluble and cell-associated factors in the differentiation process. Initially, the inventors focused on previously described factors that have been shown to play a role in early or late events in the process of dopamine neuron differentiation. Briefly, DY-1 ES cells were differentiated using EB or SDIA and candidate factors were added to the standard differentiation protocol at stages 4-5 of differentiation (Lee et al., 2000). The inventors found that Sonic Hedgehog and FGF-8 treatment led to a significant increase in either eYFP fluorescence or DAT immunoreactivity (FIG. 43). These data confirm a role for these factors (Lee et al., 2000) and validate the DY-1 assay system.

BDNF and GDNF are effective survival factors for primary dopamine neurons. GDNF is thought to increase the efficacy of dopamine transmission by increasing TH activity (Beck et al., 1996) and presynaptic release quantal size (Pothos et al., 1998), and BDNF may increase dopamine uptake (Beck et al., 1993) and turnover. Both factors appear to promote sprouting of axonal terminals. It is of interest to evaluate whether these factors may also promote the generation of functional dopamine neurons from ES cells, and whether they function at a late point in this process. The inventors find that BDNF and GDNF treatment leads to increased expression of late DN markers. However, the inventors have not yet investigated the effects of BDNF and GDNF on earlier markers in order to deduce their time of action. Furthermore, the inventors have not tested whether these factors alter cell fate decisions and survival, or the proliferation of cycling precursors.

The inventors have tested additional candidate factors that are implicated in the differentiation of dopamine neurons and other CNS neuron classes. For instance, Notch pathway plays an inhibitory role in early neuronal fate determination in uncommitted proliferative cells including neuronal stem cells (Bixby et al., 2002), as well as inhibitory roles on later events such as neurite outgrowth (Berezovska et al., 1999) in hippocampal cells. The inventors find that inhibiting Notch signaling using a soluble receptor Jagged (R&D Systems) protein leads to a significant increase in terminally differentiated dopamine neurons. Another multifunctional factor in the early and late determination of neurons is neuregulin, which inhibits neurogenesis early but subsequently plays a role promoting synapse formation at certain CNS and PNS synapses (Buonanno and Fischbach, 2001). Neuregulin1-β1 appears to increase the propensity of ES cultures to differentiate into DAT-positive cells.

Two additional pathways implicated in both early and late events in neuronal determination and maturation are the bone morphogenic proteins (BMPs) and Wnts (Goda and Davis, 2003). BMPs inhibit the generation of neuronal progenitors, in collaboration with Wnts, early in cell fate determination (Wilson et al., 2001), and provide essential signals late in differentiation (Packard et al., 2003). The inventors did not detect any effect of inhibiting BMP signaling using soluble Noggin, or of inhibiting the Wnt pathway by expression of the soluble receptor Frizzled, at the concentrations used. However, in these preliminary studies the inventors have not confirmed that the downstream signals have been appropriately altered. Also, it is possible that induction, rather than inhibition, of these pathways will modulate the generation of dopamine neurons.

Primary Mesencephalic Cultures

The differentiation of midbrain dopamine neurons in vivo is recapitulated in ES-derived cultures and in primary neuronal cultures. To extend the above analyses of dopamine neuron differentiation, and to obtain an independent assay for ‘late’ events in DN maturation, the inventors have also generated a primary mesencephlic differentiation assay. Cultures are prepared from embryonic day E13.5 CD1 mice (Staropoli et al., 2003). These cultures generate TH and DAT positive neurons over the first 7 days in culture. The inventors have described the use of lentiviral vectors to transduce greater than 95% of cells in these cultures, including primary dopamine neurons (Staropoli et al., 2003). Using the viral vectors described above, the inventors will extend the analysis of dopamine neuron differentiation using these primary cells. Also, the inventors have generated primary midbrain cultures from DY1 mice that differentiate into eYFP-positive DNs, thus allowing for the analysis of terminal dopamine neuron differentiation. These cells will allow for a detailed kinetic analysis of dopamine neuron generation using real-time imaging techniques. Also, these will be used in conjunction with the cocultures described to evaluate the effects of target and support brain cells on dopamine neuron differentiation.

Transplantation of In Vitro Generated Dopamine Neurons into Lesioned Mouse Striata

In the above studies, the inventors describe a preliminary kinetic analysis of dopamine neuron generation in vitro. A novel aspect of this study is the focus on ‘late’ events in the differentiation of dopamine neurons. Furthermore, the inventors describe a novel reagent, a fluorescent marker for ‘late’ dopamine neuron differentiation. The advantage of this in vitro approach is clearly the ability to dissect individual factors that contribute to the differentiation process and to follow the kinetics of events in a detailed manner. However, the main limitation of any in vitro approach is the lack of physiological evidence for the functional significance of such manipulations. It remains possible that an apparent alteration in a given in vitro phenotype does not correspond to an alteration in function.

The inventors have initiated studies on the efficacy of transplantation into the striatum of unilaterally 6-OHDA lesioned animals. The study protocol is essentially as described (Barberi et al., 2003; Morizane et al., 2002). 6-7 week-old male 129/sv mice (18-22 g) are housed and treated according to NIH guidelines. They are anesthesized with sodium pentobarbital (30 mg/kg) and then 0.5 ul 6-OHDA (Sigma-Aldrich; 8 ug/μl in PBS with 0.05% ascorbic acid was injected unilaterally into the striatum at the following coordinates with respect to the bregma: A +1.0, L −2.2, V −3.0 with ear bars at +0.25 using a stereotaxic apparatus for mice (Stoelting). To protect noradrenergic neurons, 30 minutes before the injection desipramine will be injected intraperitoneally at a dose of 25 mg/kg.

For transplantation, cells are trypsinized gently and resuspended in N2 media at 50,000 cells/ul. Transplantations are performed under sodium pentathol anesthesia and all surgical and animal care procedures are as according to the NIH and IACUC. Cells are transplanted using a stereotaxic apparatus into the lesioned striatum (from the bregma: A +1.0, L +2.0, V +3.0, incisor bar 0) via a Hamilton microsyringe fitted with a 26-gauge blunt needle. Successful engraftment was assessed using standard immunohistochemical methods at 4 weeks (FIG. 43).

### Example 5

Midbrain dopamine neurons (mDNs) play an essential role in the regulation of voluntary movement, and their degeneration is associated with PD. As cell replacement therapies offer a promising treatment strategy, it is of interest to identify the molecular signals that underlie mDN maturation. The ‘orphan’ nuclear receptor Nurr1 and the Paired-like homeodomain protein PitX3 are transcription factors expressed in mDNs during embryonic development (1, 2), and mouse mutant studies indicate that both genes are required for the generation of mature mDNs (3-9). Furthermore, Nurr1 can promote the expression of mDN markers, such as tyrosine hydroxylase (TH), in mouse embryonic stem (ES) cells or hippocampal neuronal precursors. However, neither Nurr1(10, 11) nor PitX3 (12) may be sufficient alone to instruct the mature mDN phenotype. Here the inventors show that Nurr1 and PitX3 collaborate to specify the ‘late’ maturation of dopamine neurons.

Nurr1-deficient mouse embryos fail to express several early mDN markers, including tyrosine hydroxylase (TH), at day 11 of gestation (E 11), but other mDN markers such as PitX3 appear normal (3-5). PAM-deficient (aphakia) mouse embryos, in contrast, harbor a complete complement of TH-positive mDNs at El 1 but these cells are subsequently lost (6-9). Thus, these transcription factors may function within independent pathways for mDN maturation, and PitX3 is likely to act at a later step (13).

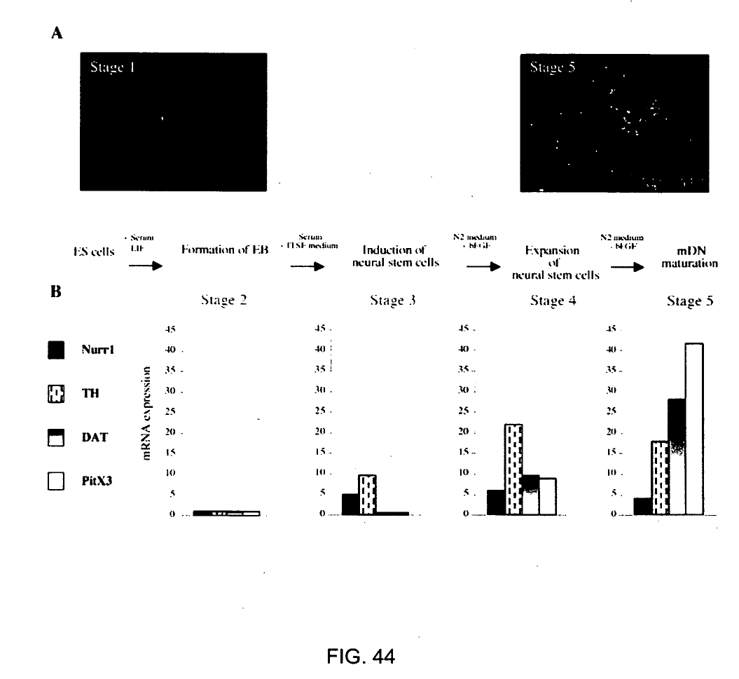

Embryonic stem cells (ES cells) are pluripotent cells that can proliferate extensively in an undifferentiated state and may provide an unlimited source of many cell types such as mDNs (11). The inventors sought to determine the roles of Nurr1 and PitX3 in the maturation events of ES-derived mDNs. A novel murine ES cell line, DY-1, was generated that harbors a Cre recombinase transgene ‘knocked-in’ at the dopamine transporter (DAT) gene locus (specifically expressed in mature mDNs) and a Cre inducible eYFP transgene at the ROSA26 locus. Differentiation of DY-1 cells in vitro using the five-stage embryoid bodies (EB) method (FIG. 44) (14) gives rise to cells that co-express eYFP and DAT, whereas eYFP is rarely detected in undifferentiated cultures (FIG. 44A). Induction of endogenous Nurr1 expression precedes that of PitX3 during EB differentiation (FIG. 44B), mimicking the in vivo expression pattern (10, 14).

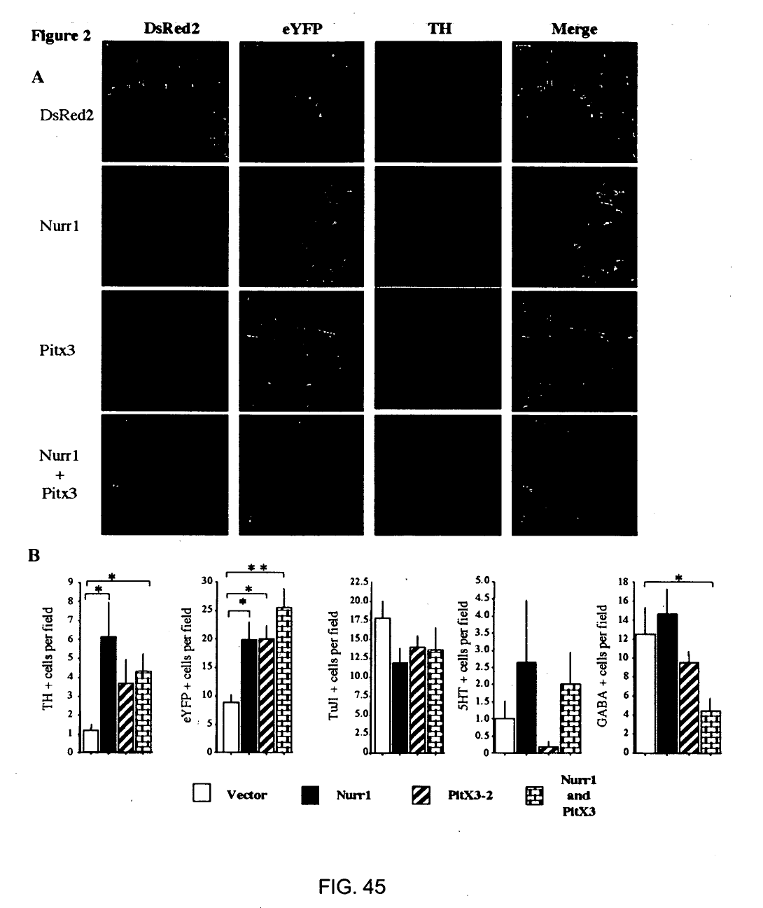

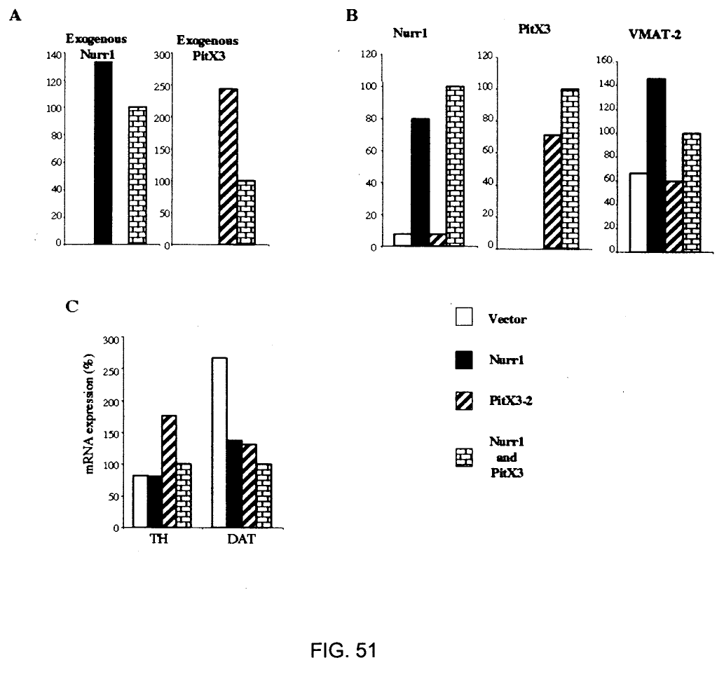

To investigate the functional role of Nurr1 and PitX3 in late events of ES cell differentiation into mDNs, DY-1 cultures were transduced with lentiviral expression vectors that harbor either the Nurr1 or PitX3 genes under the regulation of the constitutive EF1a transcriptional promoter (15). Infections were performed on DY-1 cells at the end of EB Stage 3 to approximate the normal temporal expression of these genes. Lentiviral transduction led to persistent overexpression throughout EB differentiation in over 95% of cells (FIGS. 45A and 51A). Overexpression of Nurr1, but not of PitX3 or control vector (harboring the DsRed2 fluorescent marker protein), significantly increased the number of TH+ cells (representing relatively immature mDNs; FIGS. 45A and 45B), as previously reported (11, 12, 16). Surprisingly, PitX3 was effective in the induction of YFP+ cells (representing mature DAT+ mDNs; FIGS. 45A and 45B). Furthermore, the effects of Nurr1 and PitX3 appeared to be additive on eYFP expression (FIGS. 45A and 45B). Similar results were obtained by immunostaining for endogenous DAT in an unrelated ES cell line, MM13 (FIG. 51), and thus this is not a consequence of the DY-1 transgene. In contrast, neither Nurr1 nor PitX3 altered the overall number of neurons, as determined by TuJI staining, or the number of serotonergic cells (FIGS. 45B and 51). GABAergic neuron staining appeared to be significantly reduced in the PitX3 and Nurr1 doubly transduced EB-differentiated DY-1 cultures, consistent with a cell fate switch (FIG. 45B).

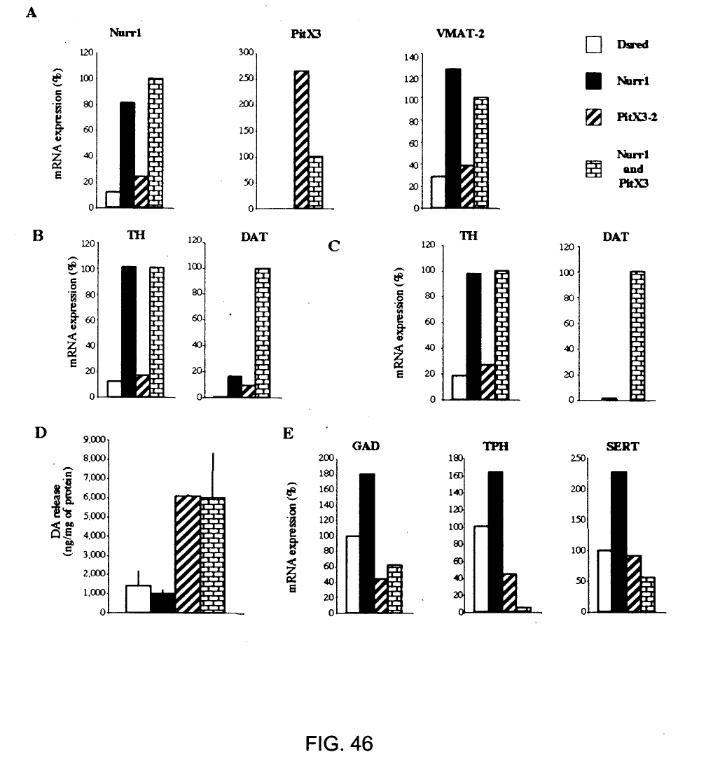

To further establish the roles of PitX3 and Nurr1 on the differentiation of ES cultures, quantitative real-time rtPCR was performed on EB-differentiated, virally transduced DY-1 cultures. Overexpression of Nurr1 resulted in the increased transcription of several relatively early mDN markers such as TH, c-Ret and VMAT-2 (FIGS. 46A, 46B, and 51), as well as a modest induction of the late marker DAT (FIG. 46B). Similarly, overexpression of PitX3 alone led to minimal induction of TH, c-Ret, or VMAT-2, as well as the late marker DAT. Strikingly, coordinate overexpression of these two transcription factors led to a dramatic induction of DAT (FIG. 46B) in either the DY-1 cultures or in an independent mouse ES cell line, MM13 (FIG. 46C). These data are consistent with the immunohistochemical study and suggest that these two factors function synergistically in the late differentiation of dopamine neurons. Demonstrating that the effects of Nurr1 and PitX3 correspond to maturation of mDN differentiation, rather than a specific effect on DAT expression, the co-expression of these factors results in a significant induction of another marker, the tyrosinase related protein 1(TyRP1). Although the exact role of this enzyme in the dopaminergic neurons is still controversial, several studies have shown the expression of tyrosinase gene, its protein and its catalytic activity in the substantia nigra (Xu, 1997 #65)(Rios, 1999 #66)(Hasegawa, 2003 #67).

In parallel, released dopamine levels were quantified in EB-differentiated cultures transduced with either PitX3, Nurr1, or both. Extracellular dopamine levels were significantly elevated in the doubly infected cultures as well as those transduced with PitX3 alone (FIG. 46D), consistent with a role for PitX3 in late DN maturation events.

Coordinate overexpression of Nurr1 and PitX3 did not significantly alter the expression of glutamic acid decarboxylase (GAD, for glutamatergic neurons), tryptophan hydroxylase (TPH, for serotonergic neurons) or the serotonin transporter (SERT, also for serotonergic neurons; FIG. 46D). In Contrast, Nurr1 alone did lead to a modest induction of expression of these non-mDN markers; thus co-expression of Nurr1 and PitX3 again appears to alter the cell fate of the EB-differentiated cultures towards mDNs. Analysis of endogenous Nurr1 and PitX3 gene expression in EB differentiated cultures indicated that there does not appear to be significant cross-talk between these pathways, as Nurr1 transduction failed to induce PitX3 expression and vice versa, consistent with the in vivo analysis of mouse mutants (FIGS. 46A and 51B) (11, 17). Overexpression of Nurr1 and PitX3 (or both) failed to induce dopamine neuron markers in undifferentiated DY-1 cultures (FIG. 51C). Thus, Nurr1 and PitX3 appear to play distinct and cooperative roles in the late differentiation of mDNs in ES differentiation.

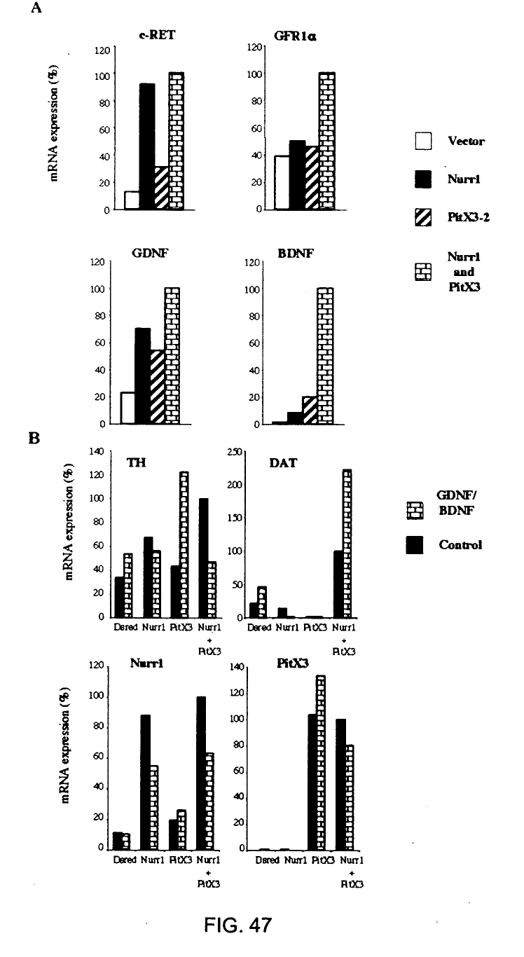

The EB differentiation protocol requires several characterized soluble factor activities (FIG. 44), and thus the inventors hypothesized that Nurr1 and PitX3 may function to alter the susceptibility of ES cells to extrinsic stimuli in these cultures. Prior reports (18) and the gene expression profiling studies of EB-differentiated cultures (data not shown) suggested that these transcription factors may regulate components of the GDNF and neurotrophin signaling pathways, which are thought to act late in the maturation process of mDNs (14, 19, 20). Consistent with this, real-time rtPCR of EB-differentiated cultures revealed that coordinate overexpression of PitX3 and Nurr1 significantly induced the expression of c-Ret and GFRa, components of the GDNF receptor complex (FIG. 47A). Furthermore, expression of the soluble factors GDNF and BDNF was also induced, suggesting the induction of an autocrine signaling loop in these cultures. Individually, Nurr1 or PitX3 overexpression did lead to some induction of a subset of these components (FIG. 47A).

To further examine the roles of Nurr1 and PitX3 in the context of these extrinsic soluble factors, the inventors exposed virally transduced EB-differentiated cultures to BDNF and GDNF. Interestingly, coordinate overexpression of PitX3 and Nurr1 expanded the population of cells sensitive to these factors, as quantified by the induction of the ‘late’ marker DAT (FIG. 47B) but not the ‘early’ marker TH. In contrast, cells transduced with both PitX3 and Nurr1 were not sensitive to the ‘early’ factors bFGF or Sonic Hedgehog and FGF-8 (data not shown). Furthermore, BDNF/GDNF treatment did not induce the expression of endogenous Nurr1 or PitX3, consistent with the notion that these transcription factors lie upstream in a signaling cascade (FIG. 47B).

To confirm the efficacy of Nurr1 and PitX3 transduction on dopamine neuron function in vivo, double-infected ES cells differentiated to EB stage 3 were transplanted into adult mice that had previously been lesioned unilaterally in the striatum with the dopamine neuron toxin 6-hydroxydopamine (6-OHDA). These animals display a characteristic turning behavior (away from the side of the lesion) in the context of the dopamine receptor agonist apomorphine as a result of denervation-induced hypersensitivity on the lesioned side (21). Transplantation of EB-differentiated ES cells that have been transduced with PitX3 and Nurr1 led to significantly greater reduction of this turning behavior than did control vector-transduced cells (FIG. 48). Immunohistochemical analysis confirmed the presence of mature transplanted cells that express both DAT and TH at the site of transplantation. Furthermore, TH immunoreactivity was increased at the site of the graft in the context of the Nurr1 and PitX3 transduced cells relative to vector alone (FIGS. 48A-B).

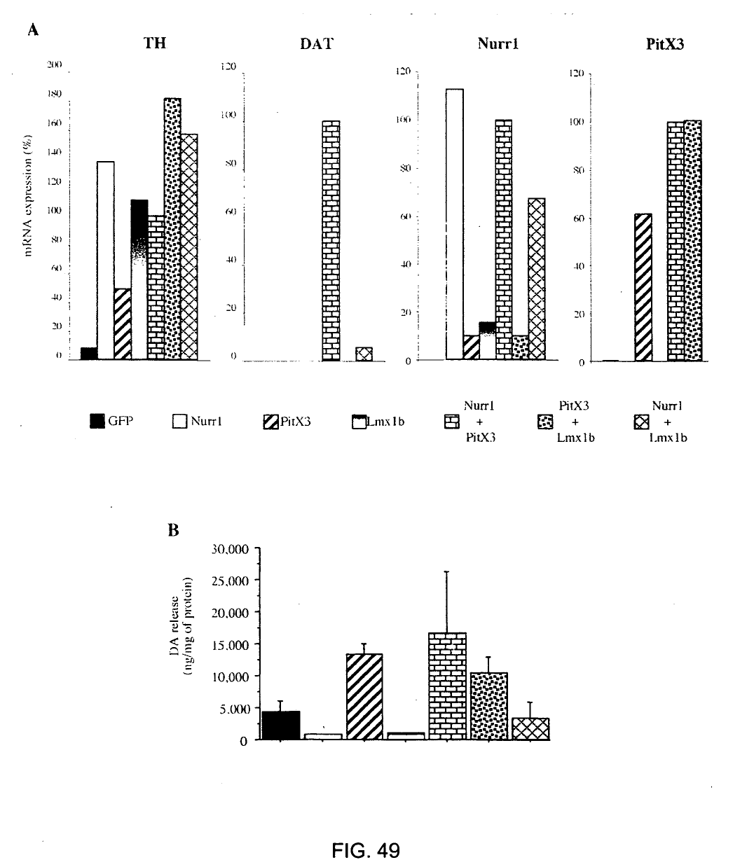

Lmx1b-deficient mice failed to express PitX3 whereas a small amount of TH and Nurr 1-positive cells were still detected at E12.5, but entirely disappeared a few days later. Although the exact role of Lmx1b in the development of mDNs remains unclear, this data suggests that Lmx1b could, together with PitX3, constitutes a molecular cascade independent of Nurr1 for the development of mDNs (Smidt, 2000 #43). The overexpression of Lmx1b alone or in conjunction with Nurr1 and PitX3 resulted in a significant increase of TH expression confirming the role of Lmx1b in the early stage of mDNs (FIG. 49). However, co-expression of Lmx1b and PitX3 did not affect both the expression of the late marker DAT and the release of dopamine (FIGS. 49A and 49B). This result indicates that PitX3 and Lmx1b are not sufficient to induce a complete maturation of ES cells-derived DNs. Surprisingly, Lmx1b transduction failed to induce PitX3 expression suggesting that Lmx1b does not control directly the expression of PitX3.

These results indicate that PitX3 and Nurr1 collaborate to promote late events in the maturation of mDNs in ES cultures. The inventors further provide evidence that Nurr1 and PitX3 act to alter cellular responsiveness to extrinsic stimuli such as GDNF; these data suggest a possible explanation for the relatively late demise of mDNs in PitX3-deficient mice. Finally, the inventors propose that transduction of transcription factor combinations such as Nurr1 and PitX3 may significantly enhance the efficacy of cell therapies for Parkinson's disease.

Materials and Methods

Generation of DY-1 ES Cells

Homozygous ‘knock-in’—mice that harbor the Cre recombinase gene at the DAT locus were bred with mice in which a Cre-inducible eYFP fluorescent marker gene was ‘knock in’ at the ROSA26 locus (22-24) to obtain double-transgenic blastocysts. An ES cell line (DY-1) was derived from these blastocysts using standard techniques (25). This cell line was demonstrated to be totipotent by injection into blastocysts and generation of ES derived chimeric animals that efficiently transmit both transgenes through the germline (data not shown).

Es Cells Culture and In Vitro Differentiation

Mouse ES cells (MM13 or DY-1) were propagated and differentiated as described previously (FIG. 44) (14) (Martinat et al., PloS, in the press). To induce EB formation (stage 2), the cells were dissociated to a single-cell suspension by Trypsin (0.025%, Specialty Media) and plated onto nonadherent bacterial dishes at a density of 2-2.5×10⁴ cells/cm² in ES media without LIF. Four-day-old EBs were plated onto adherent tissue culture surface in ES medium for one day. The selection of nestin-positive cells (stage 3) was initiated by replacing the ES medium with a serum-free insulin/transferring/selenium (ITSF) medium (26). After 6-10 days of selection, cells were dissociated by Trypsin (0.025%, Specialty Media) and plated on tissue culture dishes or glass coverslips at a concentration of 1-1.5×10⁵ cells/cm² in N2 medium (27) containing 1 μg/ml of laminin (Sigma) and 10 ng/ml of basic FGF (bFGF; Invitrogen). Before cell plating, tissue surface and glass coverslips were precoated with polyornithine (6 μg/ml, Sigma) and laminin (3 μg/ml, Sigma). Neural precursors were expanded for 6-8 days (stage 4). Terminal differentiation (stage 5) was induced by withdrawal of bFGF in N2 medium supplemented with 200 μM ascorbic acid (AA; Sigma).

Recombinant Lentiviral Vectors

The recombinant pTRIP lentiviral vectors have been described (15). Nurr1 and PitX3 ADD Lmx1b cDNAs were cloned by standard PCR methods from a human cDNA library, and the cDNAs were inserted into the pTRIP vector under the regulation of the EF1a Promoter (Martinat et al. 2004. in the press). Lmx1b was cloned from a chick cDNA library. Viral particles were pseudotyped with the vesicular stomatitis virus G protein and produced as described (28, 29). Neural precursors (stage 3) were trypsinized and single cell-suspensions (1x10⁵ cells) were infected at a multiplicity of infection of approximately 1:1.

Immunocytochemistry

Cultured cells were fixed in 4% paraformaldehyde in PBS and were incubated with the primary antibodies at 4° C. for 12 h. The following primary antibodies were used: mouse anti-TuJ1 (Covance, dilution 1/500), rabbit anti-TH (Pel-Freez, dilution 1/1000), sheep anti-TH (Pel-Freez, dilution 1/1000), rat anti-DAT (Chemicon, dilution 1/500), rabbit anti-GABA (Chemicon, dilution 1/1000), rabbit anti-5HT (Sigma, dilution 1/5000), rabbit anti-GFP (Molecular probes, dilution 1/1000). Appropriate H1 C, Cy3 and Cy5 labeled secondary antibodies (Jackson ImmunoResearch) were used.

Quantitative Real-Time PCR

Total RNA from ES cells differentiated was prepared using the absolutely RNA Miniprep kit (Stratagene). cDNA was synthesized using the SuperScript first strand synthesis system for RT-PCR (Invitrogen) and random hexamers. Real-time PCR reactions were optimized to determine the linear amplification range.

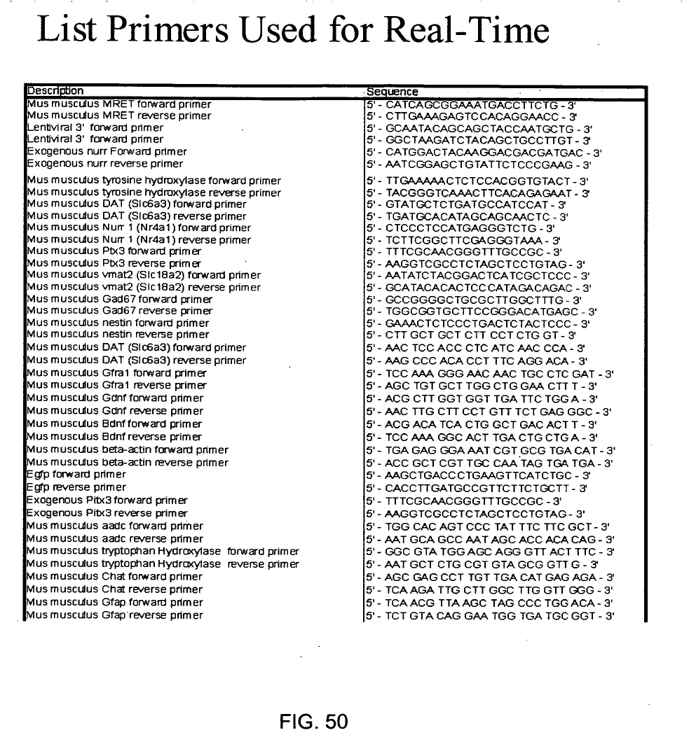

Quantitative real-time RT-PCRs were performed in Stratagene MX3000P using the QuantiTect SYBR Green PCR Master Mix (Qiagen) according to the manufacturer's instructions. For quantification analyses, PCR products levels were measured in real time during the annealing step, and values were normalized to those of (3-actin. Primer sequences are provided as FIG. 50.

HPLC Analysis

Extracellular dopamine levels were measured by HPLC. Briefly, cells were treated with 100 uM of L-DOPA for 1 hour at 37° C. and then stimulated with high KCl media (56 mM) for 15 min at 37° C. Media were collected and solution of 0.2M perchloric acid (PCA) was added to a final concentration of 0.1M PCA. These samples were centrifugated and analyzed by HPLC-EC on an ESA Coulochem HPLC as described (Martinat et al., PLoS in the press). The results were normalized to the amount of protein as determined using the BCA protein assay reagent (Pierce).

6-ORDA Lesioning, Transplantation, and Behavioral Analysis.

See Example 6.

### Example 6

Vectors

The inventors herein describe both Adeno-associated virus (AAV) and lentiviral constructs that overexpress either DJ-1, Parkin, or both. The AAV vectors are multi-component AAV-2 based vectors that have been modified to express both the gene of interest and a fluorescent protein (eGFP) under the regulation of a constitutive promoter that allows for expression in neurons, the EF 1 alpha promoter.

An additional set of vectors allow for RNAi mediated knockdown of genes of interest. These vectors use the shRNA technology to express small hairpins that are homologous to genes of interest under the regulation of the U6 promoter. This is placed within the Mlu1 site of an AAV2 vector which expresses the eGFP gene under the ef1 alpha promoter.

The vectors are then packaged in 293T cells that have been modified to express the adenoviral E1 region and co-transfected with other AAV vector components (Stratagene). Viruses are purified using a Virakit™ AAV (Virapur LLC, San Diego, Calif.; see www.virapur.com for more information). Additional purification and concentration is obtained by a 30 minutes centrifugation at 10.000 g of the purified virus through YM-10 Microcon centrifugal filter device (Millipore Corp. Bedford, Mass.). Lentiviral vector generation for overexpression or for RNAi mediated knockdown are as previously described above.

Genes

Vector inserts useful for overexpression of genes for protecting dopamine neurons include human DJ-1, Parkin, Pink1 or combinations of these genes. Vector inserts to knock-down or otherwise reduce the expression of toxic genes that have been implicated in neurodegenerative disorders include α-Synuclein specific vectors using shRNA vectors as described above, or amyloid precursor protein (APP) specific shRNA vectors. These vectors have been constructed by inserting shRNA sequences that are specific to genes of interest under the regulation of the U6 promoter.

Vector inserts to improve the efficacy of existing animal models that overexpress disease genes are aimed at inhibiting the, degradation of the disease proteins. The inventors have focused on improving two types of disease models, transgenic mice that overexpress a mutant form of alpha synuclein (A53T mutant alpha synuclein) under the regulation of the PDGF promoter (that allows expression throughout the CNS; (Giasson et al., 2002) to mimic Parkinson's disease; and transgenic mice that overexpress amyloid precursor protein (APP) (Mucke et al., 2000). These mouse models fail to accurately recapitulate the disease process. For instance, the-A-53T Synuclein mice do not display loss of dopamine neurons. To improve the efficacy of these mouse models (so that they more accurately recapitulate the disease process) the inventors have generated shRNA vectors that alter the cellular degradation machinery by targeting essential components of either proteasomal, autophagy, or vacuolar degradation pathways. This is achieved by shRNA virus-mediated knockdown of essential genes in these pathways. For instance, the inventors have knocked down two proteasomal components—PAD 1, and Psmc4 an autophagy gene, Apg7L; and a component of the lysosomal/endosomal degradation pathway—the Neimann Pick-C gene, NPC. These viral vectors thus slow the degradation and increase the efficacy of the overexpressing transgenics, allowing for more accurate recapitulation of the disease process.

Animals

Procedures involving the animal and their care are in conformity with the Columbia University Animal Protocols, in compliance with the guidelines of the National Institute of Health. Male and female mice (4 to 8 weeks) are housed at a constant temperature (23° C.) with a fixed 12 hrs light/dark cycle and have ad libidum access to food and water. All the injection procedures are done under a laminar flow hood located in the eye institute animal facility.

6-OHDA Lesioning

Procedures involving animal care were in conformity with the Columbia University Animal Protocols that are in compliance with the guidelines of the National Institute of Health. Adult male CD-1 mice (6-8 weeks; Charles River Laboratories) were housed at a constant temperature (23° C.) with a fixed 12 hrs light/dark cycle and had ad libidum access to food and water. Animals were anaesthetized with Ketamine and Xylazine (60 mg/kg and 10 mg/kg, respectively) and placed in a stereotactic frame (Stoelting). The dopamine denervation was achieved by injecting 6-OHDA (2 mg/ml in normal saline with 0.02% ascorbic acid; Sigma) in the left striatum (anterior 1 mm; lateral 2.2 mm; ventral 3 mm) as determined from the bregma and the skull surface. The 6-OHDA solution was infused at the rate of 0.5 μl/min using a 33-gauge Hamilton microsyringe. The needle was left in position for an additional 5 min before removal.

Preparation of the Cells and Graft

Stage 3 EB-differentiated ES cells transduced with GFP or Nurr1/PitX3 were washed twice with PBS, dissociated with trypsin (Gibco), and resuspended in DMEM-F12 media (Gibco). 2 ul of the cell suspension (1×10′ cells/ul) were injected in the striatum (performed as for 6-OHDA injection).

Apomorphine Turning Behavior

Apomorphine-induced turning behavior was assessed at two weeks after the 6-OHDA injection and prior to grafting, and again 6 weeks after the cell grafting (1). Mice were placed in hemispheric bowls and left for 20 min to habituate to the new environment. Apomorphine was injected subcutaneously (0.1 mg/kg or 0.4 mg/kg). Mice were videotaped and the number of turns was counted during a 7 min period by an independent observer blinded to the experimental design. Data were analyzed by the Mann-Whitney test using Statview software.

Viral Injection

Animals are anaesthetized by intramuscular injection of a mix Ketamine/Xylazine (60 mg/kg and 10 mg/kg, respectively) and placed in the mouse adaptor of a stereotactic frame (Stoelting Co., Wood Dale, Ill.). The virus is injected at different stereotactic coordinates corresponding to different anatomical brain structures (cortex, striatum, hippocampus, substantia nigra pars compacta), these coordinates being determined from the bregma, for anteriority and laterality, and the scull surface, for the depth, according to the Franklin and Paxinos mouse brain stereotactic atlas^{x1}. Injection is done using a 33-gauge needle connected to a 10.1 Hamilton® microsyringe. A motorized micropump (Stoelting Co., Wood Dale, Ill.) allows control of the volume and the rate of injection. The injection is started five minutes after the needle is on the site and the needle is left in position for additional 5 min before removal once the infusion is over. After surgery, mice are housed in the Eye Institute animal facility for 2 to 8 weeks.

Tissue Processing

Mice are anesthetized with a lethal injection of Pentobarbital, at the dose of 30 mg/kg and perfused transcardiacly with 15 ml of sterile 0.9% NaCl solution followed by 35 ml of 4% PFA pH 7.4, 4° C., at the approximate rate of 5 ml/min. Brains are removed from the scull and stored in 4% PFA at 4° C. for additional 24 h. Brains are then transferred in 0.1M PBS for 1 h and 40 microns coronal sections are processed through the whole anteroposteriority using a vibratome (Leica). Sections are either processed for immunohistochemistry or stored in 0.1 M PBS at 4° C. for up to 2 months.

Analyses

The efficacy of the protective viral vectors are quantified in either genetic or toxin animal models of PD or AD. The genetic models are described above (transgenic animals). The toxin model employed here is the unilaterally lesioned 6-OHDA mouse model as described. The viral vectors are introduced either before or after the toxic lesion to demonstrate efficacy. The efficacy of the genetic modification of cell therapies using these vectors is as described in the manuscript for Nurr1 and PitX3; the genes are introduced using viral vectors or transfection methods into the cells prior to transplantation into lesioned animals.

While the foregoing invention has been described in some detail for purposes of clarity and understanding, it will be appreciated by one skilled in the art, from a reading of the disclosure, that various changes in form and detail can be made without departing from the true scope of the invention in the appended claims.

What is claimed is:

1. A cultured cell transduced by a composition, the composition comprising:
(a) a nucleic acid encoding a Nurr-1-associated agent and a PitX3-associated agent;
(b) a vector; and
(c) optionally, a pharmaceutically-acceptable carrier;
wherein the Nurr-1-associated agent is selected from the group consisting of a Nurr-1 protein, a Nurr-1 mimetic, a modulator of Nurr-1 expression and a modulator of Nurr-1 activity; and the PitX3-associated agent is selected from the group consisting of a PitX3 protein, a PitX3 mimetic, a modulator of PitX3 expression and a modulator of PitX3 activity.

2. The cell of claim 1, wherein the vector expresses a fluorescent protein and is selected from the group consisting of an adeno-associated viral vector and a lentiviral vector.

3. The cell of claim 1, wherein the cell is selected from the group consisting of an embryonic stem (ES) cell, a neuronal precursor cell, an ES-derived dopamine neruron, a dopamine neuron precursor, and a midbrain dopamine neuron.

4. A method for treating or preventing neurodegeneration in a subject in need of treatment, comprising administering to the subject at least one cell of claim 1, in an amount effective to treat or prevent the neurodegeneration in the subject.

5. The method of claim 4, wherein the neurodegeneration is selected from the group consisting of sporadic Parkinson's disease, autosomal recessive early-onset Parkinson's disease, Alzheimer's disease, stroke, amyotrophic lateral scelerosis, Binswanger's disease, Huntington's chorea, multiple sclerosis, myasthenia gravis and Pick's disease.

6. The method of claim 4, wherein the cell is administered directly into the brain of a subject.

7. The method of claim 6, wherein the composition is administered to a brain structure selected from the group consisting of ventral midbrain, substantia nigra, hippocampus, striatum and cortex.

8. The method of claim 6, wherein the composition is administered using a stereotactic device.

9. A cultured cell transduced by a composition, the composition comprising:
(a) a nucleic acid encoding a parkin-associated agent;
(b) a vector; and
(c) optionally, a pharmaceutically-acceptable carrier;
wherein the parkin-associated agent is selected from the group consisting of a parkin protein, a parkin mimetic, a modulator of parkin expression, and a modulator of parkin activity.

10. The cell of claim 9, wherein the vector expresses a fluorescent protein and is selected from the group consisting of an adeno-associated viral vector and a lentiviral vector.

11. The cell of claim 9, wherein the cell is selected from the group consisting of an embryonic stem (ES) cell, a neuronal precursor cell, an ES-derived dopamine neruron, a dopamine neuron precursor, and a midbrain dopamine neuron.

12. A method for treating or preventing neurodegeneration in a subject in need of treatment, comprising administering to the subject at least one cell of claim 9, in an amount effective to treat or prevent the neurodegeneration in the subject.

13. The method of claim 12, wherein the neurodegeneration is selected from the group consisting of sporadic Parkinson's disease, autosomal recessive early-onset Parkinson's disease, Alzheimer's disease, stroke, amyotrophic lateral scelerosis, Binswanger's disease, Huntington's chorea, multiple sclerosis, myasthenia gravis, and Pick's disease.

14. The method of claim 12, wherein the cell is administered directly into the brain of a subject.

15. The method of claim 14, wherein the composition is administered to a brain structure selected from the group consisting of ventral midbrain, substantia nigra, hippocampus, striatum, and cortex.

16. The method of claim 14, wherein the composition is administered using a stereotactic device.

17. A cultured cell transduced by a composition, the composition comprising
(a) a nucleic acid encoding a pink1-associated agent;
(b) a vector; and
(c) optionally, a pharmaceutically-acceptable carrier;
wherein the pink1-associated agent is selected from the group consisting of a pink1 protein, a pink1 mimetic, a modulator of pink1 expression, and a modulator of pink1 activity.

18. The cell of claim 17, wherein the vector expresses a fluorescent protein, and the vector is selected from the group consisting of an adeno-associated viral vector or a lentiviral vector.

19. The cell of claim 17, wherein the cell is selected from the group consisting of an embryonic stem (ES) cell, a neuronal precursor cell, an ES-derived dopamine neuron, a dopamine neuron precursor and a midbrain dopamine neuron.

20. A method for treating or preventing neurodegeneration in a subject in need of treatment, comprising administering to the subject at least once cell of claim 17, in an amount effective to treat or prevent the neurodegeneration in the subject.

21. The method of claim 20, wherein the neurodegeneration is selected from the group consisting of sporadic Parkinson's disease, autosomal recessive early-onset Parkinson's disease, Alzheimer's disease, stroke, amyotrophic lateral scelerosis, Binswanger's disease, Huntington's chorea, multiple sclerosis, myasthenia gravis and Pick's disease.

22. The method of claim 20, wherein the composition is administered directly into the brain of a subject.

23. The method of claim 22, wherein the composition is administered to a brain structure selected from the group consisting of substantia nigra, hippocampus, striatum and cortex.

24. The method of claim 22, wherein the composition is administered using a stereotactic device.

25. A cultured cell transduced by a composition, the composition comprising:
(a) a nucleic acid encoding a pink 1-associated agent;
(b) a vector; and
(c) optionally, a pharmaceutically-acceptable carrier;
wherein the pink 1-associated agent is selected from the group consisting of a pink 1 protein, a pink 1 mimetic, a modulator of pink 1 expression and a modulator of pink 1 activity.

26. The cell of claim 25, wherein the vector expresses a fluorescent protein, and the vector is selected from the group consisting of an adeno-associated viral vector or a lentiviral vector.

27. A method for treating or preventing neurodegeneration in a subject in need of treatment, comprising administering to the subject at least one cell of claim 25, in an amount effective to treat or prevent the neurodegeneration in the subject.

28. The method of claim 27, wherein the neurodegeneration is selected from the group consisting of sporadic Parkinson's disease, autosomal recessive early-onset Parkinson's disease, Alzheimer's disease, stroke, amyotrophic lateral scelerosis, Binswanger's disease, Huntington's chorea, multiple sclerosis, myasthenia gravis and Pick's disease.

29. The method of claim 27, wherein the composition is administered directly into the brain of a subject.

30. The method of claim 29, wherein the composition is administered to a brain structure selected from the group consisting of substantia nigra, hippocampus, striatum and cortex.

31. The method of claim 27, wherein the composition is administered using a stereotactic device.

32. A cultured cell transduced by a composition, the composition comprising:
(a) a nucleic acid comprising a sequence sufficiently complementary to a portion of an alpha synuclein gene to reduce expression of the gene;
(b) a vector; and
(c) optionally, a pharmaceutically-acceptable carrier;
wherein the nucleic acid is selected from the group consisting interfering RNA, and shRNA.

33. The cell of claim 32, wherein the vector expresses a fluorescent protein and is selected from the group consisting of adeno-associated viral vector and lentiviral vector.

34. A method for treating or preventing neurodegeneration in a subject in need of treatment, comprising administering to the subject at least one cell of claim 32 in an amount effective to treat or prevent the neurodegeneration in the subject.

35. The method of claim 34, wherein the neurodegeneration is selected from the group consisting of sporadic Parkinson's disease, autosomal recessive early-onset Parkinson's disease, Alzheimer's disease, stroke, amyotrophic lateral scelerosis, Binswanger's disease, Huntington's chorea, multiple sclerosis, myasthenia gravis and Pick's disease.

36. The method of claim 34, wherein the composition is administered directly into the brain of a subject.

37. The method of claim 36, wherein the composition is administered to a brain structure selected from the group consisting of ventral midbrain, substantia nigra, hippocampus, striatum and cortex.

38. The method of claim 36, wherein the composition is administered using a stereotactic device.

39. A cultured cell transduced by a composition, the composition comprising:
(a) a nucleic acid comprising a sequence sufficiently complementary to a portion of a gene encoding amyloid precursor protein to reduce expression of the gene;
(b) a vector; and
(c) optionally, a pharmaceutically-acceptable carrier;
wherein the nucleic acid is selected from the group consisting of interfering RNA, and shRNA.

40. The cell of claim 39, wherein the vector expresses a fluorescent protein, and the vector is selected from the group consisting of an adeno-associated viral vector or a lentiviral vector.

41. A method for treating or preventing neurodegeneration in a subject in need of treatment, comprising administering to the subject at least one cell of claim 39 in an amount effective to treat or prevent the neurodegeneration in the subject.

42. The method of claim 41, wherein the neurodegeneration is selected from the group consisting of sporadic Parkinson's disease, autosomal recessive early-onset Parkinson's disease, Alzheimer's disease, stroke, amyotrophic lateral scelerosis, Binswanger's disease, Huntington's chorea, multiple sclerosis, myasthenia gravis and Pick's disease.

43. The method of claim 41, wherein the composition is administered directly into the brain of a subject.

44. The method of claim 43, wherein the composition is administered to a brain structure selected from the group consisting of ventral midbrain, substantia nigra, hippocampus, striatum and cortex.

45. The method of claim 43, wherein the composition is administered using a stereotactic device.

46. A cultured cell transduced by a composition, the composition comprising:
(a) a nucleic acid comprising a sequence sufficiently complementary to a portion of a park8 gene to reduce expression of the gene;
(b) a vector; and
(c) optionally, a pharmaceutically-acceptable carrier;
wherein the nucleic acid is selected from the group consisting interfering RNA, and shRNA.

47. The cell of claim 46, wherein the vector expresses a fluorescent protein, and the vector is selected from the group consisting of adeno-associated viral vector or lentiviral vector.

48. A method for treating or preventing neurodegeneration in a subject in need of treatment, comprising administering to the subject at least one cell of claim 46, in an amount effective to treat or prevent the neurodegeneration in the subject.

49. The method of claim 48, wherein the neurodegeneration is selected from the group consisting of sporadic Parkinson's disease, autosomal recessive early-onset Parkinson's disease, Alzheimer's disease, stroke, amyotrophic lateral scelerosis, Binswanger's disease, Huntington's chorea, multiple sclerosis, myasthenia gravis and Pick's disease.

50. The method of claim 49, wherein the composition is administered directly into the brain of a subject.

51. The method of claim 50, wherein the composition is administered to a brain structure selected from the group consisting of ventral midbrain, substantia nigra, hippocampus, striatum and cortex.

52. The method of claim 50, wherein the composition is administered using a stereotactic device.

53. A cultured cell transduced by a composition, the composition consisting:
(a) a nucleic acid comprising a sequence sufficiently complementary to a portion of a park8 gene to reduce expression of the gene;
(b) a vector; and
(c) optionally, a pharmaceutically-acceptable carrier;
wherein the nucleic acid is selected from the group consisting interfering RNA and shRNA.

54. The cell of claim 53, wherein the vector expresses a fluorescent protein.

55. A method for treating or preventing neurodegeneration in a subject in need of treatment, comprising administering to the subject at least one cell of claim 53 in an amount effective to treat or prevent the neurodegeneration in the subject.

56. The method of claim 55, wherein the neurodegeneration is selected from the group consisting of sporadic Parkinson's disease, autosomal recessive early-onset Parkinson's disease, Alzheimer's disease, stroke, amyotrophic lateral scelerosis, Binswanger's disease, Huntington's chorea, multiple sclerosis, myasthenia gravis and Pick's disease.

57. The method of claim 56 wherein the composition is administered directly into the brain of a subject.

58. The method of claim 56, wherein the composition is administered to a brain structure selected from the group consisting of ventral midbrain, substantia nigra, hippocampus, striatum and cortex.

59. The method of claim 57, wherein the composition is administered using a stereotactic device.

60. The cell of claim 55, wherein the vector expresses a fluorescent protein, and the vector is selected from the group consisting of an adeno-associated viral vector or a lentiviral vector.

61. A method for treating or preventing neurodegeneration in a subject in need of treatment, comprising administering to the subject at least one cell of claim 55 in an amount effective to treat or prevent the neurodegeneration in the subject.

62. The method of claim 61, wherein the neurodegeneration is selected from the group consisting of sporadic Parkinson's disease, autosomal recessive early-onset Parkinson's disease, Alzheimer's disease, stroke, amyotrophic lateral scelerosis, Binswanger's disease, Huntington's chorea, multiple sclerosis, myasthenia gravis and Pick's disease.

63. The method of claim 61, wherein the composition is administered directly into the brain of a subject.

64. The method of claim 63, wherein the composition is administered to a brain structure selected from the group consisting of ventral midbrain, substantia nigra, hippocampus, striatum and cortex.

65. The method of claim 63, wherein the composition is administered using a stereotactic device.

66. A cultured cell transduced by a composition, the composition consisting:
(a) a nucleic acid comprising a sequence sufficiently complementary to a portion of a gene selected from the group consisting of PAD1, Psmc4, Apg7L and NPC, to reduce expression of the gene;
(b) a vector; and
(c) optionally, a pharmaceutically-acceptable carrier;
wherein the nucleic acid is selected from the group consisting interfering RNA and shRNA.

67. The cell of claim 66, wherein the vector expresses a fluorescent protein, and the vector is selected from the group consisting of an adeno-associated viral vector or a lentiviral vector.

68. The cell of claim 66 in an animal model of neurodegeneration.

69. Use of the cell of claim 76, wherein the animal model of neurodegeneration is selected from the group consisting of sporadic Parkinson's disease, autosomal recessive early-onset Parkinson's disease, Alzheimer's disease, stroke, amyotrophic lateral scelerosis, Binswanger's disease, Huntington's chorea, multiple sclerosis, myasthenia gravis and Pick's disease.

70. A method for preparing a preparation of mature dopamine neurons comprising:
(a) contacting a cell sample with at least one fluorescent compound that targets the dopamine transporter protein;
(b) identifying cells containing the dopamine transporter protein, and removing cell and cellular aggregates that do not contain the dopamine transporter protein by fluorescent activated cell sorting (FACS) analysis; wherein the resulting preparation consists essentially of cells containing the dopamine transporter protein.

71. The method of claim 70, wherein at least one fluorescent compound is JHC 1-64.

72. The method of claim 70, wherein the cell sample contains cells selected from the group consisting of: embryonic stem (ES) cells, neuronal precursor cells, ES derived dopamine neurons, dopamine neuron precursor cells, and dopamine neurons.
